(19) United States
(12) Patent Application Publication
(10) Pub. No.: US 2026/0232701 A1
(43) Pub. Date: Aug. 13, 2026

(54) PSILOCYBIN AND AN ADJUNCTIVE SEROTONIN REUPTAKE INHIBITOR FOR USE IN THE TREATMENT OF TREAMENT- RESISTANT DEPRESSION

(71) Applicant: Compass Pathfinder Limited, Altrincham, Cheshire (GB)

(72) Inventors: Hans Åke ERIKSSON, Altrincham (GB); Ekaterina MALIEVSKAIA, Altrincham (GB)

(21) Appl. No.: 18/718,103

(22) PCT Filed: Dec. 9, 2022
(86) PCT No.: PCT/US2022/052368

§ 371 Date: Jun. 10, 2024

Related U.S. Application Data
(60) Provisional application No. 63/288,938, filed on Dec. 13, 2021.

(51) Int. Cl. A61K 31/675; A61K 31/135; A61K 31/138; A61K 31/343; A61K 31/4525; A61K 31/496; A61P 25/24

(52) U.S. Cl. CPC A61K 31/675; A61K 31/135; A61K 31/138; A61K 31/343; A61K 31/4525; A61K 31/496; A61P 25/24

(57) ABSTRACT

The disclosure provides methods for treating a subject in need thereof comprising administering to the subject a therapeutically-effective dose of psilocybin. The methods described herein may be used to treat a variety of diseases, disorders, and conditions. For example, the methods may be used to treat depression, neurocognitive disorders, autism spectrum disorder, and/or attention-deficit/hyperactivity disorder.

### CROSS-REFERENCE TO RELATED APPLICATION

The present application is a National Stage Entry of International Patent Application No. PCT/US2022/052368, filed Dec. 9, 2022, which claims the benefit of U.S. Provisional Application 63/288,938 filed on Dec. 13, 2021, the contents of which are hereby incorporated by reference in their entireties.

### BACKGROUND

Depression is one of the most common mental illnesses, affecting more than 264 million people worldwide. It is characterized by depressed mood and markedly diminished interest or pleasure in activities. Other symptoms include significant weight loss or weight gain, decrease or increase in appetite, insomnia or hypersomnia, psychomotor agitation or retardation, fatigue or loss of energy, feelings of worthlessness or excessive or inappropriate guilt, diminished ability to think or concentrate or indecisiveness, recurrent thoughts of death, suicidal ideation or suicidal attempts.

Current treatments for depression often consist of a combination of psychotherapy and one or more daily medications that regulate neurotransmitters such as dopamine, serotonin, and norepinephrine. These medications often take weeks to months to achieve their full effects and in the meantime, individuals continue to suffer from their symptoms and be at risk of self-harm, as well as harm to their personal and professional lives.

There remains a need in the art for an effective treatment for depression that provides a rapid onset of antidepressant effects within hours or a few days and is sustained long-term.

### SUMMARY

The present disclosure provides methods of treating treatment-resistant depression in a subject in need thereof.

In some embodiments, the methods described herein comprise treating treatment-resistant depression in a subject in need thereof, comprising administering an effective amount of psilocybin or an active metabolite thereof to the subject as an adjunctive to Selective Serotonin Reuptake Inhibitor (SSRI) therapy.

In some embodiments, the SSRI is selected from the group consisting of escitalopram, sertraline, fluoxetine, vilazodone, vortioxetine, paroxetine or citalopram.

In some embodiments, the SSRI is administered prior to administration of psilocybin. In some embodiments, the SSRI is administered after administration of psilocybin. In some embodiments, the SSRI is administered on the same day as the psilocybin.

In some embodiments, about 25 mg of psilocybin or an active metabolite thereof is administered to the subject.

In some embodiments, at least one sign or symptom of depression is reduced by the administration of psilocybin. In some embodiments, the sign or symptom of depression is depressed mood, diminished interest in activities, weight loss or gain, decrease or increase in appetite, insomnia or hypersomnia, psychomotor agitation or retardation, fatigue or loss of energy, feelings of worthlessness or excessive or inappropriate guilt, diminished ability to concentrate or indecisiveness, or suicidal ideation or behavior.

In some embodiments, the sign or symptom of depression is measured according to a diary assessment, an assessment by clinician or caregiver, a clinical rating scale, or by functional MRI. In some embodiments, the clinical rating scale is a Quick Inventory of Depressive Symptomatology (QIDS)-16 scale, a QIDS-16 daily scale, a Hamilton Depression Rating scale, a Beck Depression Inventory scale, a Montgomery-Asberg Depression Rating Scale (MADRS), a Clinical Global Impression Scale, a Zung Self-Rating Depression Scale, a Raskin Depression Rating Scale, and/or Young Mania Rating Scale.

In some embodiments, the clinical rating scale is MADRS. In some embodiments, the administration of psilocybin provides an about 50% reduction in patient's MADRS total score compared to prior to treatment with psilocybin. In some embodiments, the administration of psilocybin provides a reduction in the patient's MADRS total score to ≤10.

In some embodiments, the clinical rating scale is Clinical Global Impression-Severity scale (CGI-S). In some embodiments, the administration of psilocybin provides a reduction in the patient's CGI-S score to ≤2. In some embodiments, the clinical rating scale is Clinical Global Impression-Improvement scale (CGI-I). In some embodiments, the administration of psilocybin provides a reduction in the patient's CGI-I score reported as “very much improved” or “much improved” compared to prior to treatment with psilocybin.

In some embodiments, the administration of psilocybin provides a reduction in the patient's anxiety compared to prior to treatment with psilocybin. In some embodiments, the administration of psilocybin provides a reduction in the patient's Generalised Anxiety Disorder-7 item scale (GAD-7) total score compared to prior to treatment with psilocybin.

In some embodiments, at least one sign or symptom of depression is alleviated within 24 hours of administration of the psilocybin. In some embodiments, at least one symptom of depression is alleviated within 1 week of administration of the psilocybin. In some embodiments, at least one symptom of depression is alleviated for a period of at least 1 month after administration of the psilocybin. In some embodiments, at least one symptom of depression is alleviated for a period of at least 3 months after administration of the psilocybin. In some embodiments, at least one symptom of depression is alleviated for a period of at least 12 months after administration of the psilocybin.

In some embodiments, at least two doses of psilocybin are administered to the subject. In some embodiments, the psilocybin is administered once per day. In some embodiments, the psilocybin is administered at least once per week. In some embodiments, the psilocybin is administered at least twice per week. In some embodiments, the psilocybin is administered at least once per month. In some embodiments, the psilocybin is administered at least twice per month. In some embodiments, the psilocybin is administered at least once every three months. In some embodiments, the psilocybin is administered at least once every six months. In some embodiments, the psilocybin is administered at least once every 12 months. In some embodiments, each dose of psilocybin administered is about 25 mg.

In some embodiments, the psilocybin is administered by one of the following routes: oral, intravenous, intramuscular, parenteral, topical, inhalation, rectal, transmucosal, intranasal, buccal, vaginal, intrathecal, intraocular, transdermal, in utero, intralymphatic, or by direct tissue or organ injection. In some embodiments, the psilocybin is administered orally.

In some embodiments, the psilocybin comprises a crystalline polymorph of psilocybin. In some embodiments, the psilocybin comprises a crystalline polymorph of psilocybin. In some embodiments, the crystalline Polymorph A of psilocybin is characterized by XRPD peaks at 11.5±0.1, 12.0±0.1, 14.5±0.1, 17.5±0.1 and 19.7±0.1 °2θ. In some embodiments, the Polymorph A is further characterized by at least one peak selected from the group consisting of 20.4±0.1, 22.2±0.1, 24.3±0.1, and 25.7±0.1 °2θ.

In some embodiments, the crystalline psilocybin comprises Hydrate A. In some embodiments, the Hydrate A is characterized by X-ray powder diffraction (XRPD) peaks at 8.9±0.1, 13.8±0.1, 19.4±0.1, 23.1±0.1 and 23.5±0.1 °2θ. In some embodiments, the Hydrate A is further characterized by at least one peak selected from the group consisting of 6.5±0.1, 12.2±0.1, 12.6±0.1, 16.2±0.1, 20.4±0.1, 20.8±0.1, and 21.5±0.1 °2θ.

In some embodiments, the psilocybin has no single impurity of greater than 1% as determined by HPLC analysis. In some embodiments, the psilocybin has a chemical purity of greater than 97% as determined by HPLC analysis.

### BRIEF DESCRIPTION OF THE DRAWINGS

FIG. 1 is a numbered structural formula of psilocybin.

FIG. 2A is a XRPD diffractogram of Polymorph A (GM764B).

FIG. 2B is a XRPD diffractogram of Polymorph A′ (JCCA2160F).

FIG. 2C is a XRPD diffractogram of Polymorph B (JCCA2160-F-TM2).

FIG. 2D is a XRPD diffractogram of a Hydrate A (JCCA2157E).

FIG. 2E is a XRPD diffractogram of an ethanol solvate (JCCA2158D).

FIG. 2F is a XRPD diffractogram of product obtained during development of the process (CB646-E) (top)—compared to the diffractograms Polymorph A′ (JCCA2160F) (middle) and Polymorph B (JCCA2160-TM2) (bottom).

FIG. 3A is a DSC and TGA thermograph of Polymorph A (GM764B).

FIG. 3B is a DSC and TGA thermograph of Polymorph A′ (JCCA2160F).

FIG. 3C is a DSC thermograph of Polymorph B (GM748A).

FIG. 3D is a DSC and TGA thermograph of Hydrate A (JCCA2157E).

FIG. 3E is a DSC and TGA thermograph of ethanol solvate (JCCA2158D).

FIG. 4 is a form phase diagram showing the inter-relationship of form in water-based systems.

FIG. 5 is a 1H NMR (Nuclear Magnetic Resonance) spectrum of psilocybin.

FIG. 6 is a 13C NMR spectrum of psilocybin.

FIG. 7 is a FT-IR Spectrum of psilocybin.

FIG. 8 is a Mass Spectrum of psilocybin.

FIG. 9 shows the study design of the clinical trial examining psilocybin as an adjunctive therapy in participants with treatment-resistant depression.

FIG. 10 shows the patient disposition from the clinical trial examining psilocybin as an adjunctive therapy in participants with treatment-resistant depression.

FIG. 11 shows the SSRIs that patients were taking at the time psilocybin was administered in the clinical trial examining psilocybin as an adjunctive therapy in participants with treatment-resistant depression.

FIG. 12A shows the mean change from baseline in Montgomery-Asberg Depression Rating Scale (MADRS) total score observed during the clinical study described in Example 2.

FIG. 12B shows the mean MADRS total score observed during the clinical study described in Example 2.

FIG. 13 shows the proportion of MADRS responders by visit observed during the clinical study described in Example 2.

FIG. 14 shows the proportion of MADRS remitters by visit observed during the clinical study described in Example 2.

FIG. 15A shows the mean change from baseline in Clinical Global Impression-Severity scale (CGI-S) total score observed during the clinical study described in Example 14.

FIG. 15B shows the mean CGI-S total score observed during the clinical study described in Example 2.

FIG. 16 shows the proportion of CGI-S responders by visit observed during the clinical study described in Example 2.

FIG. 17A shows the mean change from baseline in Generalised Anxiety Disorder-7 item scale (GAD-7) total score observed during the clinical study described in Example 2.

FIG. 17B shows the mean GAD-7 total score observed during the clinical study described in Example 2.

FIG. 18A shows the mean change from baseline in Positive and Negative Affect Schedule (PANAS) Negative Affect total score and the mean PANAS Negative Affect total score observed during the clinical study described in Example 2.

FIG. 18B shows the mean change from baseline in PANAS Positive Affect total score and the mean PANAS Positive Affect total score observed during the clinical study described in Example 2.

FIG. 19 shows the summary of the 5-Dimensional Altered States of Consciousness scale (5D-ASC) at day 1 observed during the clinical study described in Example 2.

FIG. 20A shows the mean change from baseline in 16-Item Quick Inventory of Depressive Symptomatology-Self Report (QIDS-SR-16) total score observed during the clinical study described in Example 2.

FIG. 20B shows the mean QIDS-SR-16 total score observed during the clinical study described in Example 2.

FIG. 21A shows the mean change from baseline in EuroQol-5-Dimension-3-Level Scale (EQ-5D-3L) total score observed during the clinical study described in Example 2.

FIG. 21B shows the mean EQ-5D-3L total score observed during the clinical study described in Example 2.

FIG. 22A shows the mean change from baseline in Euro Qol visual analog scale (EQ-VAS) total score observed during the clinical study described in Example 14.

FIG. 22B shows the mean EQ-VAS total score observed during the clinical study described in Example 2.

FIG. 23 shows the proportion of Clinical Global Impression-Improvement scale (CGI-I) responders by visit observed during the clinical study described in Example 2.

### DETAILED DESCRIPTION

### Definitions

Unless otherwise defined, all technical and scientific terms used herein have the same meaning as commonly understood by one of ordinary skill in the art to which this disclosure belongs. The terminology used in the detailed description herein is for the purpose of describing particular embodiments only and is not intended to be limiting.

The singular forms “a,” “an” and “the” are intended to include the plural forms as well, unless the context clearly indicates otherwise.

Furthermore, the term “about” as used herein when referring to a measurable value such as a dose, time, temperature, and the like, is meant to encompass variations of ±20%, ±10%, ±5%, ±1%, ±0.5%, or even ±0.1% of the specified amount.

The phrase “and/or,” as used herein in the specification and in the embodiments, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements can optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the embodiments, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the embodiments, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the embodiments, shall have its ordinary meaning as used in the field of patent law.

As used herein in the specification and in the embodiments, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements can optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

Unless the context indicates otherwise, it is specifically intended that the various features described herein can be used in any combination.

As used herein, the terms “reduce,” “decrease,” “lessen” and similar terms mean a decrease of at least about 10%, about 15%, about 20%, about 25%, about 35%, about 50%, about 75%, about 80%, about 85%, about 90%, about 95%, about 97%, or more.

As used herein, the terms “improve,” “increase,” “enhance,” and similar terms indicate an increase of at least about 10%, about 15%, about 20%, about 25%, about 50%, about 75%, about 100%, about 150%, about 200%, about 300%, about 400%, about 500%, or more.

Reference to a particular numerical value includes at least that particular value, unless the context clearly dictates otherwise. When a range of values is expressed, another embodiment includes from the one particular value and/or to the other particular value. Further, reference to values stated in ranges include each and every value within that range. All ranges are inclusive and combinable.

As used herein, “substantially absent” with reference to XRPD diffractogram peak means the peak has a relative intensity compared to a reference peak present in the diffractogram of less than about 5%, less than about 4%, less than about 3%, less than about 2%, or less than about 1% of the intensity of the reference peak, or that the peak is not detectable.

XRPD diffractograms and XRPD peak positions may be acquired using Cu Kα radiation.

DSC thermograms and TGA thermograms may be acquired using a heating rate of 20° C./min.

As used herein, the term “diffusion tensor imaging” or “DTI” refers to a technique that detects how water travels along the white matter tracts in the brain. In some embodiments, DTI is used to characterize microstructural changes associated with mental disorders (e.g., major depressive disorder) and/or the response to treatment in subjects with mental disorders.

All disease and disorders listed herein are defined as described in the Diagnostic and Statistical Manual of Mental Disorders (DSM-5), published by the American Psychiatric Association, or in International Classification of Diseases (ICD), published by the World Health Organization.

As used herein the term “subject” and “patient” are used interchangeably.

As used herein, “treating” and like terms refer to reducing the severity and/or frequency of one or more symptoms, eliminating one or more symptoms and/or the underlying cause of said symptoms, reducing the frequency or likelihood of one or more symptoms and/or their underlying cause, delaying, preventing and/or slowing the progression of diseases and/or disorders and improving or remediating damage caused, directly or indirectly, by the diseases and/or disorders.

As used herein, “therapeutically-effective dose” means a dose sufficient to achieve the intended therapeutic purpose, such as, to alleviate a sign or symptom of a disease or disorder in a subject.

As used herein a “precursor” and/or “derivative” of psilocybin includes, but is not limited to, prodrugs of psilocybin, prodrugs of an active metabolite of psilocybin, and an active metabolite of psilocybin.

As used herein, a subject that is “psilocybin-naïve” has not previously been exposed to psilocybin.

As used herein, the following Medical Dictionary for Regulatory Activities (MedDRA) terms are considered to be adverse events that are psychedelic in nature: altered mood, altered state of consciousness, autoscopy, delusional perception, disinhibition, dissociation, dissociative identity disorder, dreamy state, emotional disorder, euphoric mood, feeling abnormal, hallucination, hyperacusis, hyperaesthesia, hypoaesthesia, illusion, paranoia, parosmia, photophobia, sensory disturbance, time perception altered, thinking abnormal, synaesthesia, substance-induced psychotic distress, and somatic hallucination.

### Depressive Disorders

In some embodiments, the methods provided herein are used to treat a subject with a depressive disorder. As used herein, the terms “depressive disorder”, “depression disorder”, or “depression” refers to a group of disorders characterized by low mood that can affect a person's thoughts, behavior, feelings, and sense of well-being lasting for a period of time. In some embodiments, the depressive disorder disrupts the physical and psychological functions of a person. In some embodiments, the depressive disorder causes a physical symptom such as weight loss, aches or pains, headaches, cramps, or digestive problems. In some embodiments, the depressive disorder causes a psychological symptom such as persistent sadness, anxiety, feelings of hopelessness and irritability, feelings of guilt, worthlessness, or helplessness, loss of interest or pleasure in hobbies and activities, difficulty concentrating, remembering, or making decisions.

In some embodiments, the depressive disorder is major depressive disorder, atypical depression, bipolar disorder, catatonic depression, depressive disorder due to a medical condition, postpartum depression, premenstrual dysphoric disorder, or seasonal affective disorder.

As used herein, the term “major depressive disorder” refers to a condition characterized by a time period of low mood that is present across most situations. Major depressive disorder is often accompanied by low self-esteem, loss of interest in normally enjoyable activities, low energy, and pain without a clear cause. In some instances, major depressive order is characterized by two weeks. In some instances, an individual experiences periods of depression separated by years. In some instances, an individual experiences symptoms of depression that are nearly always present. Major depressive disorder can negatively affect a person's personal, work, or school life, as well as sleeping, eating habits, and general health. Approximately 2-7% of adults with major depressive disorder commit suicide, and up to 60% of people who commit suicide had major depressive disorder or another related mood disorder. Dysthymia is a subtype of major depressive disorder consisting of the same cognitive and physical problems as major depressive disorder with less severe but longer-lasting symptoms. Exemplary symptoms of a major depressive disorder include, but are not limited to, feelings of sadness, tearfulness, emptiness or hopelessness, angry outbursts, irritability or frustration, even over small matters, loss of interest or pleasure in most or all normal activities, sleep disturbances, including insomnia or sleeping too much, tiredness and lack of energy, reduced appetite, weight loss or gain, anxiety, agitation or restlessness, slowed thinking, speaking, or body movements, feelings of worthlessness or guilt, fixating on past failures or self-blame, trouble thinking, concentrating, making decisions, and remembering things, frequent thoughts of death, suicidal thoughts, suicide attempts, or suicide, and unexplained physical problems, such as back pain or headaches.

As used herein, the term “atypical depression” refers to a condition wherein an individual shows signs of mood reactivity (i.e., mood brightens in response to actual or potential positive events), significant weight gain, increase in appetite, hypersomnia, heavy, leaden feelings in arms or legs, and/or long-standing pattern of interpersonal rejection sensitivity that results in significant social or occupational impairment. Exemplary symptoms of atypical depression include, but are not limited to, daily sadness or depressed mood, loss of enjoyment in things that were once pleasurable, major changes in weight (gain or loss) or appetite, insomnia or excessive sleep almost every day, a state of physical restlessness or being rundown that is noticeable by others, daily fatigue or loss of energy, feelings of hopelessness, worthlessness, or excessive guilt almost every day, problems with concentration or making decisions almost every day, recurring thoughts of death or suicide, suicide plan, or suicide attempt.

As used herein, the term “bipolar disorder” refers to a condition that causes an individual to experience unusual shifts in mood, energy, activity levels, and the ability to carry out day-to-day tasks. Individuals with bipolar disorder experience periods of unusually intense emotion, changes in sleep patterns and activity levels, and unusual behaviors. These distinct periods are called “mood episodes.” Mood episodes are drastically different from the moods and behaviors that are typical for the person. Exemplary symptoms of mania, excessive behavior, include, but are not limited to, abnormally upbeat, jumpy, or wired behavior; increased activity, energy, or agitation, exaggerated sense of well-being and self-confidence, decreased need for sleep, unusual talkativeness, racing thoughts, distractibility, and poor decision-making—for example, going on buying sprees, taking sexual risks, or making foolish investments. Exemplary symptoms of depressive episodes or low mood, include, but are not limited to, depressed mood, such as feelings of sadness, emptiness, hopelessness, or tearfulness; marked loss of interest or feeling no pleasure in all—or almost all—activities, significant weight loss, weight gain, or decrease or increase in appetite, insomnia or hypersomnia (excessive sleeping or excessive sleepiness), restlessness or slowed behavior, fatigue or loss of energy, feelings of worthlessness or excessive or inappropriate guilt, decreased ability to think or concentrate, or indecisiveness, and thinking about, planning or attempting suicide.

Bipolar disorder includes bipolar I disorder, bipolar II disorder, and cyclothymic disorder. Bipolar I disorder is defined by manic episodes that last at least 7 days or by severe manic symptoms that require hospitalization. A subject with bipolar I disorder may also experience depressive episodes typically lasting at least 2 weeks. Episodes of depression with mixed features, i.e. depressive and manic symptoms at the same time, are also possible. Bipolar II disorder is characterized by a pattern of depressive and hypomanic episodes, but not severe manic episodes typical of bipolar I disorder. Cyclothymic disorder (also referred to as cyclothymia) is characterized by periods of hypomanic symptoms (elevated mood and euphoria) and depressive symptoms lasting over a period of at least 2 years. The mood fluctuations are not sufficient in number, severity, or duration to meet the full criteria for a hypomanic or depressive episode.

As used herein, the term “catatonic depression” refers to a condition causing an individual to remain speechless and motionless for an extended period. Exemplary symptoms of catatonic depression include, but are not limited to, feelings of sadness, which can occur daily, a loss of interest in most activities, sudden weight gain or loss, a change in appetite, trouble falling asleep, trouble getting out of bed, feelings of restlessness, irritability, feelings of worthlessness, feelings of guilt, fatigue, difficulty concentrating, difficulty thinking, difficulty making decisions, thoughts of suicide or death, and/or a suicide attempt.

As used herein, the term “depressive disorder due to a medical condition” refers to a condition wherein an individual experiences depressive symptoms caused by another illness. Examples of medical conditions known to cause a depressive disorder include, but are not limited to, HIV/AIDS, diabetes, arthritis, strokes, brain disorders such as Parkinson's disease, Huntington's disease, multiple sclerosis, and Alzheimer's disease, metabolic conditions (e.g. vitamin B12 deficiency), autoimmune conditions (e.g., lupus and rheumatoid arthritis), viral or other infections (hepatitis, mononucleosis, herpes), back pain, and certain cancers (e.g., pancreatic).

As used herein, the term “postpartum depression” refers to a condition as the result of childbirth and hormonal changes, psychological adjustment to parenthood, and/or fatigue. Postpartum depression is often associated with women, but men can also suffer from postpartum depression as well. Exemplary symptoms of postpartum depression include, but are not limited to, feelings of sadness, hopeless, emptiness, or overwhelmed; crying more often than usual or for no apparent reason; worrying or feeling overly anxious; feeling moody, irritable, or restless; oversleeping, or being unable to sleep even when the baby is asleep; having trouble concentrating, remembering details, and making decisions; experiencing anger or rage; losing interest in activities that are usually enjoyable; suffering from physical aches and pains, including frequent headaches, stomach problems, and muscle pain; eating too little or too much; withdrawing from or avoiding friends and family; having trouble bonding or forming an emotional attachment with the baby; persistently doubting his or ability to care for the baby; and thinking about harming themselves or the baby.

As used herein, the term “premenstrual dysphoric disorder” refers to a condition wherein an individual expresses mood lability, irritability, dysphoria, and anxiety symptoms that occur repeatedly during the premenstrual phase of the cycle and remit around the onset of menses or shortly thereafter. Exemplary symptoms of premenstrual dysphoric disorder includes, but are not limited to, lability (e.g., mood swings), irritability or anger, depressed mood, anxiety and tension, decreased interest in usual activities, difficulty in concentration, lethargy and lack of energy, change in appetite (e.g., overeating or specific food cravings), hypersomnia or insomnia, feeling overwhelmed or out of control, physical symptoms (e.g., breast tenderness or swelling, joint or muscle pain, a sensation of ‘bloating’ and weight gain), self-deprecating thoughts, feelings of being keyed up or on edge, decreased interest in usual activities (e.g., work, school, friends, hobbies), subjective difficulty in concentration, and easy fatigability.

As used herein, the term “seasonal affective disorder” refers to a condition wherein an individual experiences mood changes based on the time of the year. In some instances, an individual experiences low mood, low energy, or other depressive symptoms during the fall and/or winter season. In some instances, an individual experiences low mood, low energy, or other depressive symptoms during the spring and/or summer season. Exemplary symptoms of seasonal affective disorder include, but are not limited to, feeling depressed most of the day or nearly every day, losing interest in activities once found enjoyable, having low energy, having problems with sleeping, experiencing changes in appetite or weight, feeling sluggish or agitated, having difficulty concentrating, feeling hopeless, worthless, or guilty, and having frequent thoughts of death or suicide.

In some embodiments, a depressive disorder comprises a medical diagnosis based on the criteria and classification from Diagnostic and Statistical Manual of Medical Disorders, 5th Ed. In some embodiments, a depressive disorder comprises a medical diagnosis based on an independent medical evaluation.

In some embodiments, the methods described herein are provided to a subject with depression that is resistant to treatment. In some embodiments, the subject has been diagnosed with “treatment-resistant depression”. The term “treatment-resistant depression” refers to a kind of depression that does not respond or is resistant to at least one or more treatment attempts of adequate dose and duration. In some embodiments, the subject with treatment-resistant depression has failed to respond to 1 treatment attempt, 2 treatment attempts, 3 treatment attempts, 4 treatment attempts, or 5 treatment attempts. In some embodiments, the subject with treatment-resistant depression has been diagnosed with major depressive disorder and has failed to respond to 3 or more treatment attempts. In some embodiments, the subject with treatment-resistant depression has been diagnosed with bipolar disorder and has failed to respond to 1 treatment attempt.

In some embodiments, the methods provided herein reduce at least one sign or symptom of a depressive disorder. In some embodiments, the methods provided herein reduce at least one sign or symptom of a depressive disorder by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, or more, compared to prior to treatment.

In some embodiments, the methods provided herein reduce at least one sign or symptom of major depressive disorder. In some embodiments, the methods provided herein reduce at least one sign or symptom of major depressive disorder by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, or more, compared to prior to treatment.

In some embodiments, the methods provided herein reduce at least one sign or symptom of atypical depression. In some embodiments, the methods provided herein reduce at least one sign or symptom of atypical depression by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, or more, compared to prior to treatment.

In some embodiments, the methods provided herein reduce at least one sign or symptom of bipolar disorder. In some embodiments, the methods provided herein reduce at least one sign or symptom of bipolar disorder by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, or more, compared to prior to treatment.

In some embodiments, the methods provided herein reduce at least one sign or symptom of bipolar I disorder. In some embodiments, the methods provided herein reduce at least one sign or symptom of bipolar I disorder by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, or more, compared to prior to treatment.

In some embodiments, the methods provided herein reduce at least one sign or symptom of bipolar II disorder. In some embodiments, the methods provided herein reduce at least one sign or symptom of bipolar II disorder by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, or more, compared to prior to treatment.

In some embodiments, the methods provided herein reduce at least one sign or symptom of catatonic depression. In some embodiments, the methods provided herein reduce at least one sign or symptom of catatonic depression by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, or more, compared to prior to treatment.

In some embodiments, the methods provided herein reduce at least one sign or symptom of a depressive disorder due to a medical condition. In some embodiments, the methods provided herein reduce at least one sign or symptom of a depressive disorder due to a medical condition by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, or more, compared to prior to treatment.

In some embodiments, the methods provided herein reduce at least one sign or symptom of postpartum depression. In some embodiments, the methods provided herein reduce at least one sign or symptom of postpartum depression by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, or more, compared to prior to treatment.

In some embodiments, the methods provided herein reduce at least one sign or symptom of premenstrual dysphoric disorder. In some embodiments, the methods provided herein reduce at least one sign or symptom of premenstrual dysphoric disorder by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, or more, compared to prior to treatment.

In some embodiments, the methods provided herein reduce at least one sign or symptom of seasonal affective disorder. In some embodiments, the methods provided herein reduce at least one sign or symptom of seasonal affective disorder by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, or more, compared to prior to treatment.

In some embodiments, the sign or symptom of depression is reduced or eliminated in the subject within 1 hour, 2 hours, 3 hours, 6 hours, 12 hours, 24 hours, 48 hours, 3 days, 1 week, 2 weeks, 3 weeks, 4 weeks, 5 weeks, 6 weeks, 7 weeks, 8 weeks, or 3 months following administration of psilocybin or an active metabolite thereof.

In some embodiments, the sign or symptom of depression is reduced or eliminated in the subject for a period of 1 day, 3 days, 7 days, 10 days, 2 weeks, 3 weeks, 4 weeks, 5 weeks, 6 weeks, 7 weeks, 8 weeks, 3 months, 4 months, 5 months, 6 months, 7 months, 8 months, 9 months, 10 months, 11 months, 12 months, 18 months, 24 months, or 48 months following administration of psilocybin or an active metabolite thereof. In some embodiments, there is no recurrence of depression in the subject upon administration of psilocybin or an active metabolite thereof.

In some embodiments, no other treatment is administered to the subject to reduce the sign or symptom of depression after administration of the psilocybin.

In some embodiments, the method of the present disclosure further comprises administering to the subject at least one additional therapeutic to reduce the sign or symptom of depression. In some embodiments, at least one additional therapeutic is a selective serotonin reuptake inhibitor, a serotonin and norepinephrine reuptake inhibitor, a tricyclic antidepressant, a tetracyclic antidepressant, a dopamine reuptake inhibitor, a 5-HT1A receptor antagonist, a 5-HT2 receptor antagonist, a 5-HT3 receptor antagonist, a monoamine oxidase inhibitor, or a noradrenergic antagonist. In some embodiments, at least one additional therapeutic is administered prior to administration of psilocybin, on the same day as the administration of psilocybin, or after administration of psilocybin.

In some embodiments, the subject with the depressive disorder has an additional comorbidity or disorder. In some embodiments, the additional comorbidity or disorder is an anxiety disorder, an obsessive-compulsive disorder, alcoholism, a personality disorder, a cardiovascular disease, a neurological disease, or cancer. In some embodiments, the subject has dementia, Alzheimer's Disease, or Parkinson's Disease. In some embodiments, reducing at least one sign or symptom of depression in the subject using the methods of the present disclosure prevents one or more comorbidities or disorders in the subject.

### Psilocybin

In some embodiments, a method of treatment comprises the administration of a therapeutically effective amount of psilocybin, a prodrug of psilocybin, an active metabolite of psilocybin, or a prodrug of an active metabolite of psilocybin to a subject in need thereof as described herein. In some embodiments, a method of treatment comprises the administration of a therapeutically effective amount of psilocybin as described herein. In some embodiments, a method of treatment comprises the administration of a therapeutically effective amount of psilocin as described herein. Some embodiments comprise psilocybin, a prodrug of psilocybin, an active metabolite of psilocybin, or a prodrug of an active metabolite of psilocybin for use in the treatment of an indication as described herein. Some embodiments comprise psilocybin for use in the treatment of an indication as described herein. Some embodiments comprise psilocin for use in the treatment of an indication as described herein. Some embodiments comprise the use of psilocybin, a prodrug of psilocybin, an active metabolite of psilocybin, or a prodrug of an active metabolite of psilocybin in the manufacture of a medicament for the treatment of an indication as described herein.

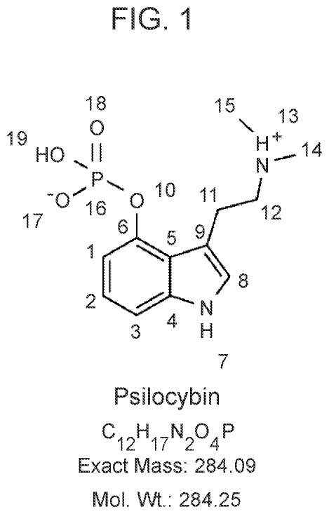

A numbered structural formula of psilocybin is shown in FIG. 1. Novel polymorphs and hydrates of psilocybin, along with the preparation and formulations thereof are disclosed in U.S Application No. US2019/0119310 A1, which is incorporated by reference herein in its entirety. US2019/0119310 discloses a number of formulations and the challenges of formulating psilocybin due to e.g. its hygroscopicity and poor flow characteristics. US2019/0119310 also discloses the importance of a controlled aqueous crystallisation process.

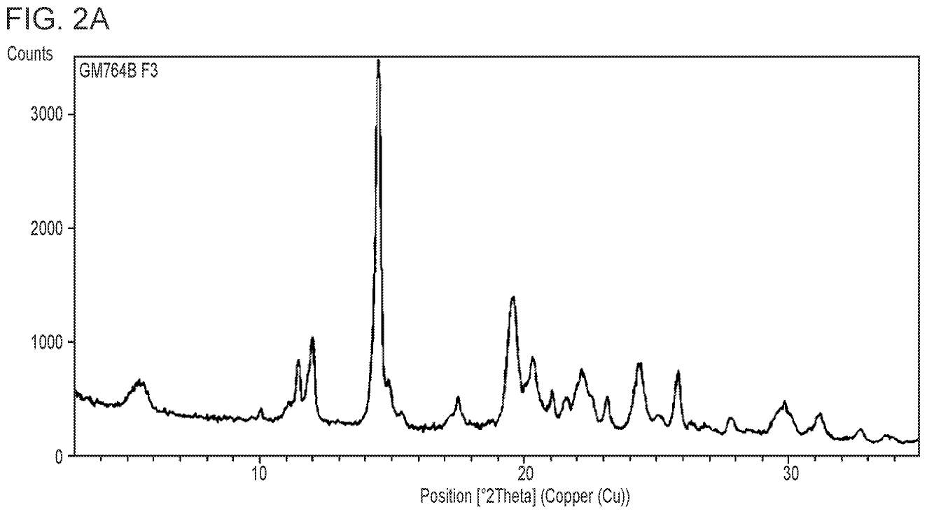

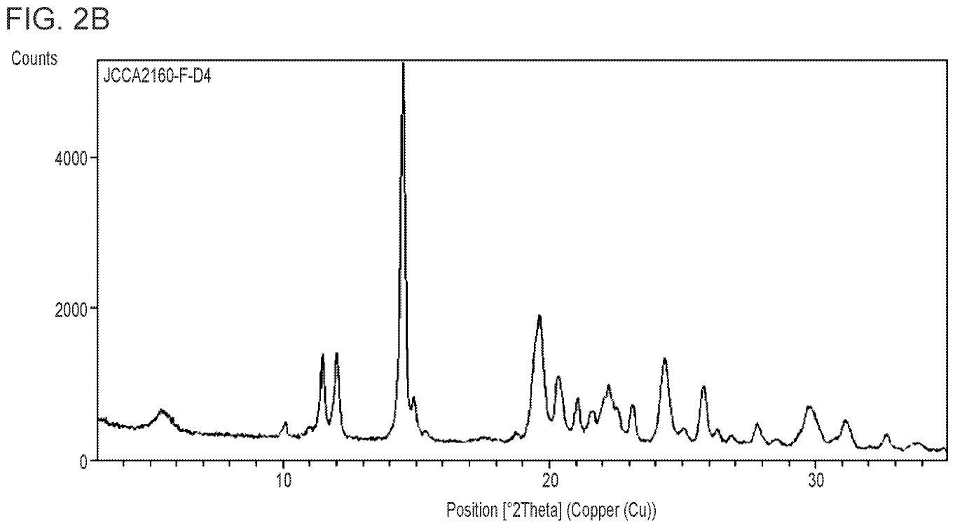

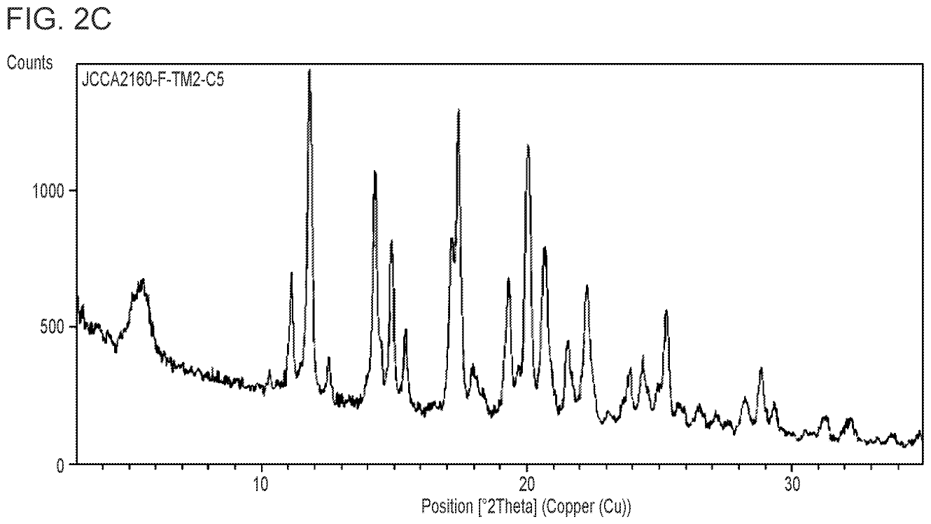

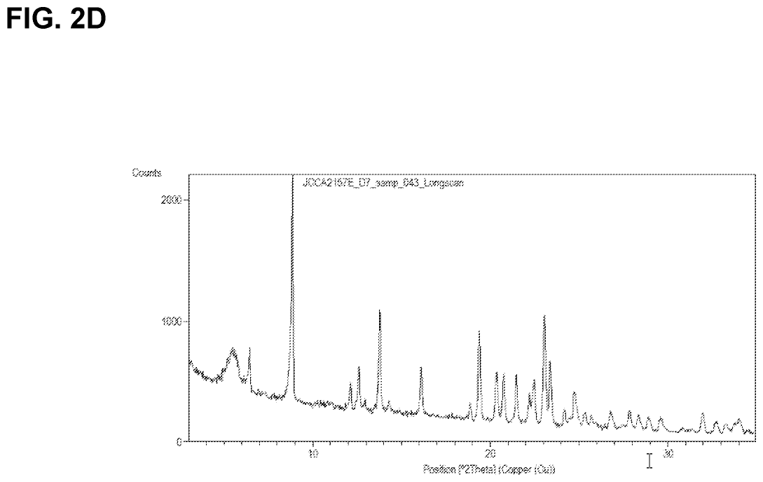

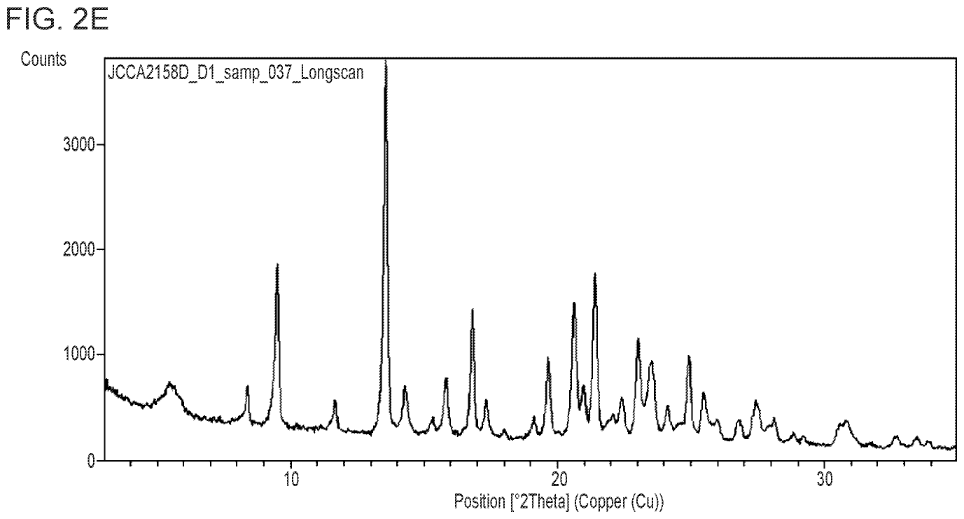

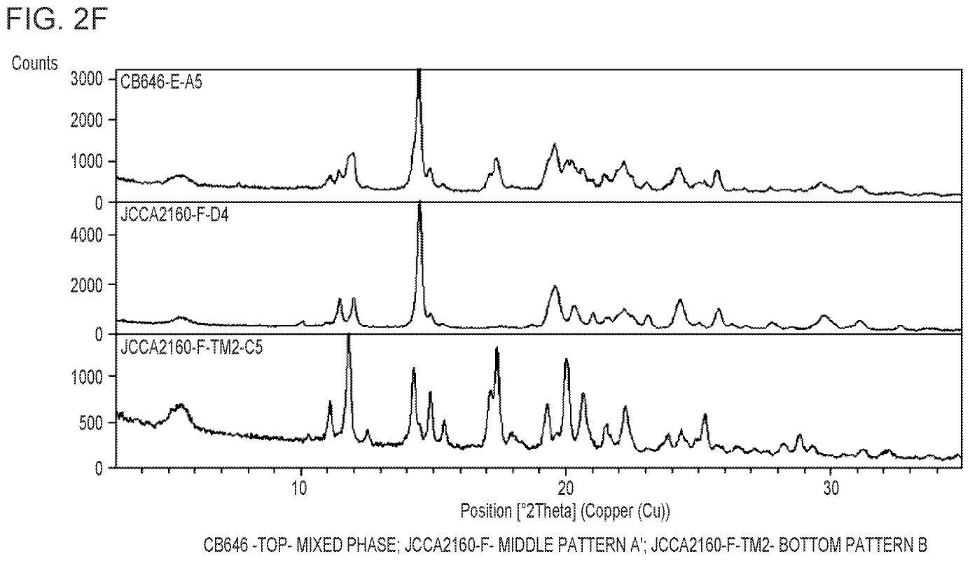

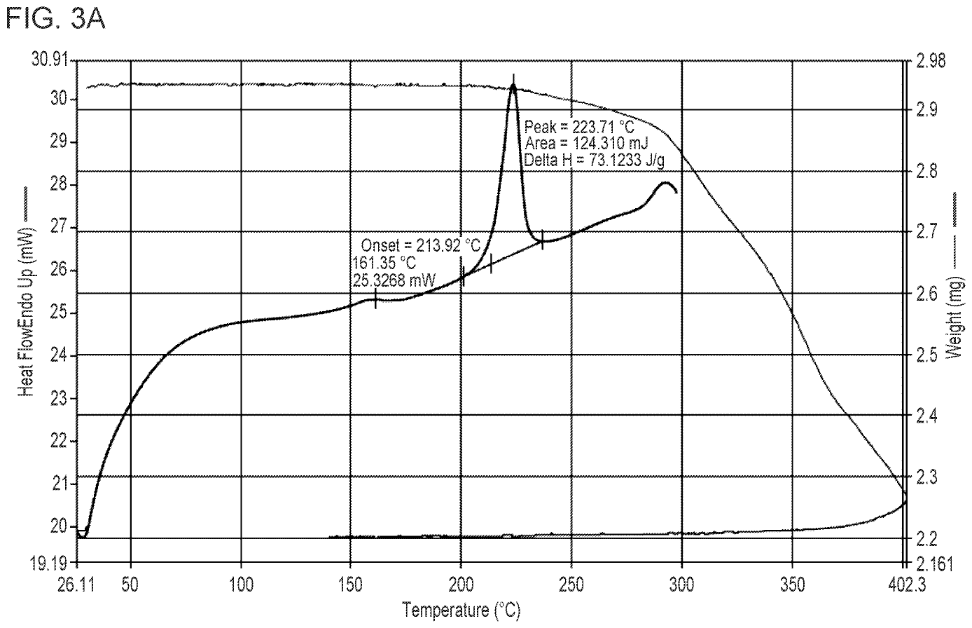

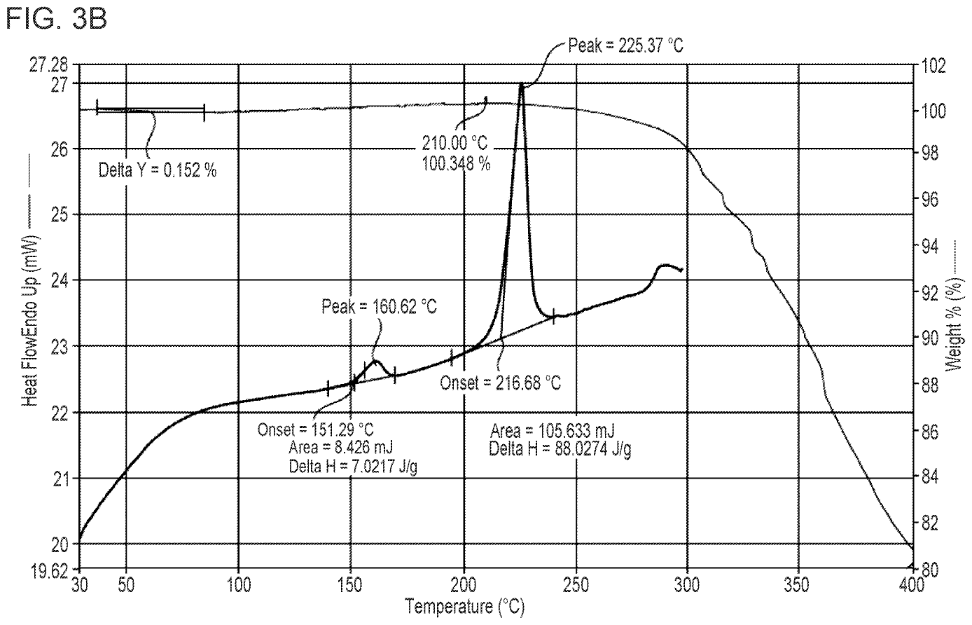

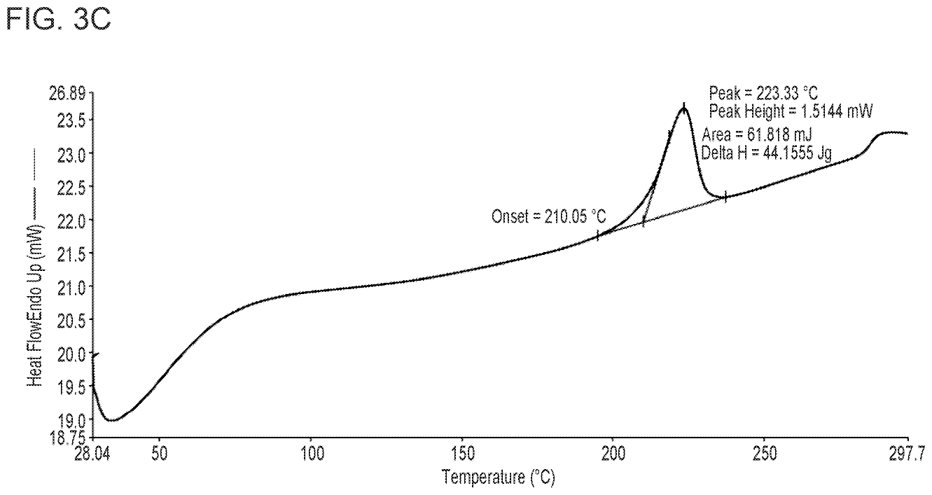

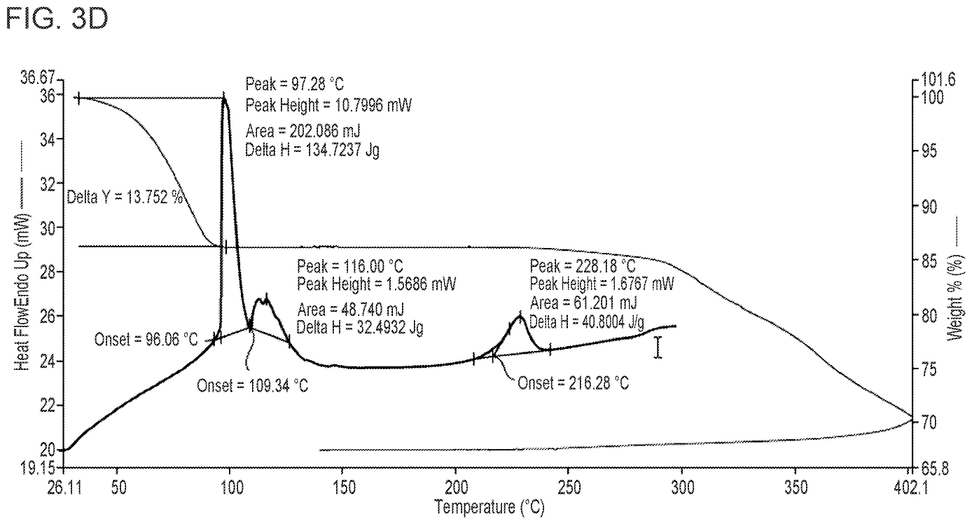

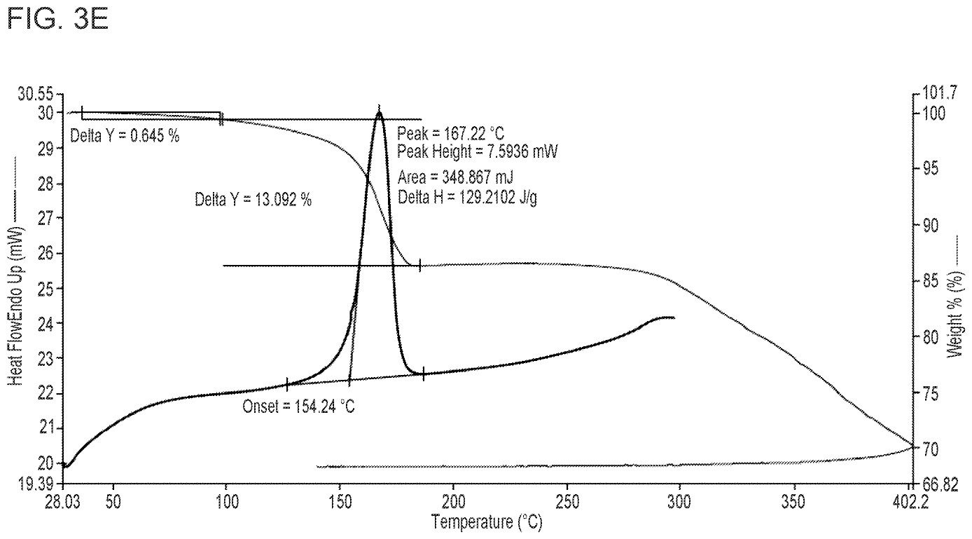

In some embodiments, the psilocybin comprises crystalline psilocybin in the form Polymorph A or Polymorph A′, as described herein, the crystalline psilocybin exhibiting peaks in an X-ray powder diffraction (XRPD) diffractogram at 11.5, 12.0 and 14.5 °2θ±0.1 °2θ. In some embodiments, the crystalline psilocybin further exhibits at least one peak in the XRPD diffractogram at 19.7, 20.4, 22.2, 24.3 or 25.7 °2θ±0.1 °2θ. Illustrative XRPD diffractograms are provided as FIGS. 2A and 2B. In some embodiments, the crystalline psilocybin exhibits an endothermic event in a DSC thermogram having a first onset temperature of between 145° C. and 165° C. and a second onset temperature of between 205° C. and 220° C. Illustrative DSC thermograms are provided as FIGS. 3A and 3B.

### Polymorph A

In some embodiments, the present disclosure provides crystalline psilocybin in the form Polymorph A, characterized by one or more of:
peaks in an XRPD diffractogram at 11.5, 12.0, 14.5, and 17.5, °2θ±0.1 °2θ;
peaks in an XRPD diffractogram at 11.5, 12.0, 14.5 and 17.5, °2θ±0.1 °2θ, further characterized by at least one further peak at 19.7, 20.4, 22.2, 24.3 or 25.7 °2θ±0.1 °2θ;
an XRPD diffractogram as substantially illustrated in FIG. 2A; or
an endothermic event in a DSC thermogram having an endothermic event in a DSC thermogram having a first onset temperature of between 145° C. and 165° C. and a second onset temperature of between 205° C. and 220° C. substantially as illustrated in FIG. 3A.

In some embodiments, the peak at 17.5 °2θ±0.1 °2θ has a relative intensity compared to the peak at 14.5 °2θ±0.1 °2θ of at least 5%, at least 6%, at least 7%, at least 8%, at least 9%, or at least 10%.

In some embodiments, the present disclosure provides crystalline psilocybin in the form Polymorph A, characterized by one or more of:
peaks in an XRPD diffractogram at 11.5, 12.0, 14.5, and 17.5, °2θ±0.2 °2θ;
peaks in an XRPD diffractogram at 11.5, 12.0, 14.5 and 17.5, °2θ±0.2 °2θ, further characterized by at least one further peak at 19.7, 20.4, 22.2, 24.3 or 25.7 °2θ±0.2 °2θ;
an XRPD diffractogram as substantially illustrated in FIG. 2A; or
an endothermic event in a DSC thermogram having an endothermic event in a DSC thermogram having a first onset temperature of between 145° C. and 165° C. and a second onset temperature of between 205° C. and 220° C. substantially as illustrated in FIG. 3A.

In some embodiments, the crystalline psilocybin of Polymorph A exhibits an XRPD diffractogram having at least 3, 4, 5, 6, 7, 8, 9, 10, 11, 12, 13, 14, 15, 16, or 17 of the peaks listed in Table A, or equivalent peaks within about ±0.1 °2θ of the peaks listed in Table A. In some embodiments, the crystalline psilocybin of Polymorph A exhibits an XRPD diffractogram having at least 3, 4, 5, 6, 7, 8, 9, 10, 11, 12, 13, 14, 15, 16, or 17 of the peaks listed in Table A, or equivalent peaks within about ±0.2 °2θ of the peaks listed in Table A. In some embodiments, Polymorph A exhibits a peak at 17.5 °2θ±0.1 °2θ that is substantially absent in Polymorph A′. In some embodiments, Polymorph A exhibits a peak at 17.5 °2θ±0.2 °2θ that is substantially absent in Polymorph A′.

TABLE A
XRPD peak positions for Polymorph A

| | Relative |
|---|---|
| Position | Intensity |
| [°2Th.] | [%] |
| 5.6 | 8.42 |
| 11.5 | 13.05 |
| 12.0 | 26.45 |
| 14.5 | 100.00 |
| 17.5 | 10.71 |
| 19.7 | 37.29 |
| 20.4 | 20.06 |
| 22.2 | 17.83 |
| 23.2 | 6.99 |
| 24.3 | 17.93 |
| 25.7 | 16.40 |
| 26.8 | 3.15 |
| 27.8 | 4.54 |
| 29.7 | 9.53 |
| 31.2 | 6.51 |
| 32.6 | 2.45 |
| 33.7 | 1.75 |

In some embodiments, crystalline psilocybin Polymorph A exhibits XRPD diffractogram peaks at 11.5, 12.0, 14.5, and 17.5 °2θ±0.1 °2θ. In some embodiments, crystalline psilocybin Polymorph A exhibits at least one additional peak appearing at 19.7, 20.4, 22.2, 24.3 or 25.7 °2θ±0.1 °2θ. In some embodiments, crystalline psilocybin Polymorph A exhibits at least two additional peaks appearing at 19.7, 20.4, 22.2, 24.3 or 25.7 °2θ±0.1 °2θ. In some embodiments, crystalline psilocybin Polymorph A exhibits at least three additional peaks appearing at 19.7, 20.4, 22.2, 24.3 or 25.7 °2θ±0.1 °2θ. In some embodiments, crystalline psilocybin Polymorph A exhibits an XRPD diffractogram substantially the same as the XRPD diffractogram shown in FIG. 2A.

In some embodiments, crystalline psilocybin Polymorph A is characterized by XRPD diffractogram peaks at 14.5 and 17.5 °2θ±0.1 °2θ with the peak at 17.5 °2θ having an intensity which is at least about 5%, at least about 6%, at least about 7%, at least about 8%, at least about 9%, or at least about 10% of the intensity of the peak at 14.5 °2θ.

In some embodiments, the crystalline psilocybin Polymorph A exhibits no peak at 10.1—that is, the peak at 10.1 is absent or substantially absent.

In some embodiments, crystalline psilocybin Polymorph A is characterized by an endothermic event in a DSC thermogram having a first onset temperature of between 145° C. and 165° C. such as between 145 and 160° C., or such as between 145 and 155° C. and a second onset temperature of between 205 and 220° C., such as between 21° and 220° C., such as between 21° and 218° C., or such as between 21° and 216° C. In some embodiments, crystalline psilocybin Polymorph A exhibits an endothermic event in a DSC thermogram having an onset temperature of between about 205 and about 220° C., between about 210 and about 220° C., between about 210 and about 218° C., or between about 210 and about 216° C. In some embodiments, crystalline psilocybin Polymorph A further exhibits an endothermic event in the DSC thermogram having an onset temperature of between about 145 and about 165° C., between about 145 and about 160° C., or between about 145 and about 155° C. In some embodiments, crystalline psilocybin Polymorph A exhibits an endothermic event having an onset temperature of between about 205 and about 220° C., between about 210 and about 220° C., between about 210 and about 218° C., or between about 210 and about 216° C.; and an endothermic event having an onset temperature of between about 145 and about 165° C., between about 145 and about 160° C., between about 145 and about 155° C., in a DSC thermogram. In some embodiments, crystalline psilocybin Polymorph A exhibits a DSC thermogram substantially the same as the DSC thermogram in FIG. 3A.

In some embodiments, crystalline psilocybin Polymorph A exhibits a water content of <0.5% w/w, <0.4% w/w, <0.3% w/w, <0.2% w/w, or <0.1% w/w. The water content of a crystalline compound can be determined by known methods, for example Karl Fischer Titration. In some embodiments, crystalline psilocybin Polymorph A exhibits <0.5% w/w loss, <0.4% w/w, <0.3% w/w, <0.2% w/w, or <0.1% w/w in the TGA thermogram between ambient temperature, e.g., about 25° C., and 200° C. In some embodiments, crystalline psilocybin Polymorph A loses less than 2% by weight, less than 1% by weight, or than 0.5% by weight in a loss on drying test, e.g., a loss on drying test performed at 70° C.

In some embodiments, crystalline psilocybin Polymorph A is a highly pure crystalline form of Polymorph A, for example, the in a loss on drying test psilocybin comprises at least 90%, at least 95%, at least 99%, or at least 99.5% by weight crystalline psilocybin of Polymorph A.

In some embodiments, crystalline psilocybin Polymorph A is a white to off-white solid.

In some embodiments, crystalline psilocybin Polymorph A is chemically pure, for example the psilocybin has a chemical purity of greater than 97%, 98%, or 99% by HPLC. In some embodiments, crystalline psilocybin Polymorph A has no single impurity of greater than 1%, greater than 0.5%, greater than 0.4%, greater than 0.3%, or greater than 0.2% e.g., the impurity phosphoric acid as measured by 31P NMR, or the impurity psilocin measured by HPLC. In some embodiments, crystalline psilocybin Polymorph A has a chemical purity of greater than 97 area %, greater than 98 area %, or greater than 99 area % by HPLC. In some embodiments, crystalline psilocybin Polymorph A has no single impurity greater than 1 area %, greater than 0.5 area %, greater than 0.4%, greater than 0.3%, or greater than 0.2% as measured by HPLC. In some embodiments, crystalline psilocybin Polymorph A does not contain psilocin at a level greater than 1 area %, greater than 0.5 area %, greater than 0.4%, greater than 0.3%, or greater than 0.2% as measured by HPLC. In some embodiments, crystalline psilocybin Polymorph A does not contain phosphoric acid at a level greater than 1 weight %, greater than 0.5 weight %, greater than 0.4 weight %, 0.3 weight %, or greater than 0.2 weight %, as measured by 31P NMR. In some embodiments, crystalline psilocybin Polymorph A has a chemical assay of at least 95 weight %, at least 96 weight %, or at least 98 weight %.

### Methods of Manufacturing Crystalline Psilocybin Polymorph A.

In another embodiment, the disclosure provides a method for large scale manufacture of psilocybin characterized in that the method comprises subjecting psilocybin to a water crystallization step, with controlled drying, to produce crystalline psilocybin Polymorph A.

In another embodiment, the disclosure provides a method for large scale manufacture of psilocybin characterized in that the method comprises subjecting psilocybin to a water crystallization step, with controlled drying, to produced crystalline psilocybin Polymorph A with an XRPD diffractogram as illustrated in FIG. 2A and a DSC and TGA thermograph as illustrated in FIG. 3a. In another embodiment, the disclosure provides a method for large-scale manufacture of psilocybin characterized in that the method comprises subjecting psilocybin to a water crystallization step, with controlled drying, to produce a high purity crystalline psilocybin-Polymorph A with an XRPD diffractogram as illustrated in FIG. 2A and a DSC thermograph as illustrated in b FIG. 3A.

In another embodiment of the disclosure, psilocybin is recrystallized in about 10-20 volumes of water, heated with agitation to a temperature of at least 70° C., polish filtered with a suitable cut off (typically, below 5 μm), seeded at a temperature of about 70° C., and cooled in a controlled manner to about 5° C. over a period of more than 2 hours.

In some embodiments, psilocybin recrystallization comprises controlled cooling which drops the temperature by about 5° C.-15° C. an hour, more preferably about 10° C. an hour. In certain embodiments, the polish filter step is done through an appropriately sized filter such as, but not limited to, a 1.2 μm in line filter.

In some embodiments, agitation is by stirring at about 400-500 rpm, typically about 450 rpm.

In some embodiments, the psilocybin is dissolved in water heated to no more than 90° C. In some embodiments the psilocybin is dissolved in water heated to no more than 85° C. Without being bound by any particular mechanism, this dissolution step is intended to solubilize psilocybin whilst also minimizing the formation of hydrolysis products.

In some embodiments, the psilocybin solution is stirred to speed the solubilization and reduce the time that the solution is at a high temperature, namely one at or around 80° C., or higher.

In some embodiments, the seed is psilocybin Hydrate A. In one embodiment, 0.1% weight or less of seed is added to the process.

In some embodiments, the psilocybin the crystalline psilocybin is isolated by vacuum filtration.

In some embodiments, the isolated crystals are dried in vacuo at a temperature of at least 30° C., such as between 30 and 50° C., or such as between 4° and 50° C. In some embodiment, the isolated crystals are dried in vacuo for at least 10 hours, such as between 12 and 18 hours, or such as about 30 hours. In some embodiments, the isolated crystals are dried in vacuo at a temperature of at least 30° C., such as between 3° and 50° C., or such as between 4° and 50° C., for at least 10 hours, such as between 12 and 18 hours, or such as about 30 hours. In some embodiments, the isolated crystals are dried until the isolated crystals lose less than 2% weight in a loss on drying test, such as less than 0.5% weight.

In some embodiments, the isolated crystals are washed, several times, in water and dried in vacuo at about 50° C. for at least 12 hours.

In some embodiments, the crystals obtained are typically relatively large (range 50 to 200 microns) and uniform when viewed under the microscope ×10.

In contrast, crystals obtained without controlled cooling which are much smaller in size (typically 5 to 50 microns) when viewed under the microscope ×10.

In some embodiments, there is provided Psilocybin obtained by the method of crystallization described herein.

In some embodiments, there is provided a pharmaceutical formulation comprising psilocybin polymorph A obtained by the method of crystallization described herein.

In some embodiments, psilocybin manufactured prior to crystallization may be produced using one of the following methods: synthetic or biological, e.g. by fermentation or obtained by extraction from mushrooms. In some embodiments, psilocybin manufactured prior to crystallization is manufactured according to all or some of the methods described in U.S Application No. US2019/0119310 A1, which is incorporated by reference herein in its entirety.

### Polymorph A′

The present disclosure provides crystalline psilocybin in the form of Polymorph A′, characterized by one or more of:
(a) (i) peaks in an XRPD diffractogram at 11.5, 12.0 and 14.5 °2θ±0.1 °2θ, but absent or substantially absent of a peak at 17.5 °2θ±0.1 °2θ;
(b) (ii) peaks in an XRPD diffractogram at 11.5, 12.0 and 14.5 °2θ±0.1 °2θ, but absent or substantially absent of a peak at 17.5 °2θ±0.1 °2θ, further characterized by at least one further peak at 19.7, 20.4, 22.2, 24.3 or 25.7 °2θ±0.1 °2θ;
(c) (iii) an XRPD diffractogram as substantially illustrated in FIG. 2B; or
(d) (iv) an endothermic event in a DSC thermogram having a first onset temperature of between 145° C. and 165° C. and a second onset temperature of between 205° C. and 220° C. substantially as illustrated in FIG. 3B.

In some embodiments, the crystalline psilocybin comprises crystalline psilocybin Polymorph A′. Crystalline psilocybin Polymorph A′ exhibits peaks in an XRPD diffractogram at 11.5, 12.0 and 14.5 °2θ±0.1 °2θ, but absent or substantially absent of a peak at 17.5 °2θ±0.1 °2θ. In some embodiments, crystalline psilocybin Polymorph A′ further exhibits 1, 2, 3, 4, or 5 peaks selected from 19.7, 20.4, 22.2, 24.3 or 25.7. °2θ±0.1 °2θ. An illustrative XRPD diffractogram for Polymorph A′ is provided as FIG. 2B. An illustrative DSC thermogram having an onset temperature of between 205 and 220° C. for Polymorph A′ is provided as FIG. 3B.

In some embodiments, psilocybin Polymorph A′ exhibits an XRPD diffractogram as summarized in Table B. In some embodiments, crystalline psilocybin Polymorph A′ exhibits at least 3, 4, 5, 6, 7, 8, 9, 10, 11, 12, 13, 14, 15, 16, 17, 18, 19, 20, 21, 22, 23, 24, or 25 peaks listed of Table B or equivalent peaks within about ±0.1 °2θ, and absent or substantially absent peak at 17.5 °2θ±0.1 °2θ.

TABLE B
XRPD peak positions for Polymorph A′

| | Relative |
|---|---|
| Position | Intensity |
| [°2Th.] | [%] |
| 5.5 | 4.89 |
| 10.1 | 4.09 |
| 11.5 | 22.05 |
| 12.0 | 22.77 |
| 14.5 | 100.00 |
| 14.9 | 11.29 |
| 17.5 | 1.08 |
| 18.7 | 2.44 |
| 19.4 | 23.02 |
| 19.6 | 33.70 |
| 20.3 | 17.01 |
| 21.1 | 12.08 |
| 21.6 | 8.51 |
| 22.2 | 15.54 |
| 22.6 | 8.78 |
| 23.1 | 10.11 |
| 24.3 | 21.83 |
| 25.1 | 4.36 |
| 25.8 | 15.40 |
| 26.3 | 4.28 |
| 26.8 | 2.86 |
| 27.8 | 5.96 |
| 28.6 | 1.91 |
| 29.7 | 10.56 |
| 31.1 | 7.35 |
| 32.6 | 3.72 |
| 33.8 | 1.54 |

In some embodiments, crystalline psilocybin Polymorph A′ exhibits XRPD diffractogram peaks at 11.5, 12.0, and 14.5 °2θ±0.1 °2θ but substantially absent of a peak at 17.5 °2θ±0.1 °2θ. In some embodiments, crystalline psilocybin Polymorph A′ further exhibits at least one additional peak appearing at 19.7, 20.4, 22.2, 24.3, or 25.7 °2θ±0.1 °2θ. In some embodiments, crystalline psilocybin Polymorph A′ exhibits at least two additional peaks appearing at 19.7, 20.4, 22.2, 24.3, or 25.7 °2θ±0.1 °2θ. In some embodiments, crystalline psilocybin Polymorph A′ exhibits and is distinguished from Polymorph A by the presence of a peak appearing at 10.1 °2θ±0.1 °2θ. In yet a further embodiment, crystalline psilocybin Polymorph A′ exhibits an XRPD diffractogram substantially the same as the XRPD diffractogram shown in FIG. 2B.

In some embodiments, crystalline psilocybin Polymorph A′ exhibits XRPD diffractogram peaks at 14.5 and 17.5 °2θ±0.1 °2θ, wherein the intensity of the peak at 17.5 °2θ is less than 5%, less than 4%, less than 3%, less than 2%, or less than 1% of the intensity of the peak at 14.5 °2θ.

In some embodiments, crystalline psilocybin Polymorph A′ exhibits XRPD diffractogram peaks at 10.1 and 14.5 °2θ±0.1 °2θ, wherein the intensity of the peak at 10.1 °2θ is at least 1%, at least 2%, at least 3%, or at least 4% of the intensity of the peak at 14.5 °2θ.

In some embodiments, crystalline psilocybin Polymorph A′ is characterized by an endothermic event in a DSC thermogram having a first onset temperature of between 145° C. and 165° C. such as between 145 and 160° C., or such as between 145 and 155° C. and a second onset temperature of between 205 and 220° C., such as between 21° and 220° C., such as between 21° and 218° C., or such as between 21° and 216° C. In some embodiments, crystalline psilocybin Polymorph A′ is characterized by an endothermic event in a DSC thermogram having an onset temperature of between about 205 and about 220° C., between about 210 and about 220° C., between about 210 and about 218° C., or between about 210 and about 216° C. In some embodiments, crystalline psilocybin Polymorph A′ exhibits an endothermic event in the DSC thermogram having an onset temperature of between about 145 and about 165° C., between about 145 and about 160° C., or between about 145 and about 155° C. In some embodiments, crystalline psilocybin Polymorph A′ exhibits an endothermic event having an onset temperature of between about 205 and about 220° C., between about 210 and about 220° C., between about 210 and about 218° C., or between about 210 and about 216° C., and an endothermic event having an onset temperature of between about 145 and about 165° C., between about 145 and about 160° C., or between about 145 and about 155° C., in a DSC thermogram. In some embodiments, crystalline psilocybin Polymorph A′ exhibits a DSC thermogram substantially the same as the DSC thermogram in FIG. 3B.

In some embodiments, crystalline psilocybin Polymorph A′ exhibits a water content of <0.5% w/w, <0.4% w/w, <0.3% w/w, <0.2% w/w, or <0.1% w/w. Methods to determine the water content of a crystalline compound are known, for example Karl Fischer Titration. In some embodiments, crystalline psilocybin Polymorph A′ exhibits <0.5% w/w loss, <0.4% w/w, <0.3% w/w, <0.2% w/w, <0.1% w/w in the TGA thermogram between ambient temperature, e.g., 25° C., and 200° C. In some embodiments, crystalline psilocybin Polymorph A′ loses less than 2% by weight, less than 1% by weight, or less than 0.5% by weight in a loss on drying test. In some embodiments, the loss on drying test is performed at 70° C.

In some embodiments, crystalline psilocybin Polymorph A′ is a highly pure crystalline form of Polymorph A′. In some embodiments, the crystalline psilocybin comprises at least 90%, 95%, 99%, or 99.5% by weight of Polymorph A′.

In some embodiments, crystalline psilocybin Polymorph A's is a white to off white solid.

In some embodiments, crystalline psilocybin Polymorph A′ is chemically pure, for example the psilocybin has a chemical purity of greater than 97%, greater than 98%, or than 99% by HPLC. In some embodiments, crystalline psilocybin Polymorph A′ has no single impurity of greater than 1% or greater than 0.5%, e.g., the impurity phosphoric acid as measured by 31P NMR or the impurity psilocin as measured by HPLC. In some embodiments, crystalline psilocybin Polymorph A′ has a chemical purity of greater than 97 area %, greater than 98 area %, or greater than 99 area % by HPLC. In some embodiments, crystalline psilocybin Polymorph A′ has no single impurity greater than 1 area % or greater than 0.5 area %, e.g., as measured by HPLC. In some embodiments, crystalline psilocybin Polymorph A′ does not contain psilocin at a level greater than 1 area % or greater than 0.5 area % as measured by HPLC. In some embodiments, crystalline psilocybin Polymorph A′ does not contain phosphoric acid at a level greater than 1 weight % or greater than 0.5 weight % as measured by 31P NMR. In some embodiments, crystalline psilocybin Polymorph A′ has a chemical assay of at least 95 weight %, at least 96 weight %, or at least 98 weight %.

In some embodiments, crystalline psilocybin Polymorph A′ is chemically pure, for example the psilocybin has a chemical purity of greater than 97%, 98%, or 99% by HPLC. In some embodiments, crystalline psilocybin Polymorph A′ has no single impurity of greater than 1%, greater than 0.5%, greater than 0.4%, greater than 0.3%, or greater than 0.2% e.g., the impurity phosphoric acid as measured by 31P NMR, or the impurity psilocin measured by HPLC. In some embodiments, crystalline psilocybin Polymorph A′ has a chemical purity of greater than 97 area %, greater than 98 area %, or greater than 99 area % by HPLC. In some embodiments, crystalline psilocybin Polymorph A′ has no single impurity greater than 1 area %, greater than 0.5 area %, greater than 0.4%, greater than 0.3%, or greater than 0.2% as measured by HPLC. In some embodiments, crystalline psilocybin Polymorph A′ does not contain psilocin at a level greater than 1 area %, greater than 0.5 area %, greater than 0.4%, greater than 0.3%, or greater than 0.2% as measured by HPLC. In some embodiments, crystalline psilocybin Polymorph A′ does not contain phosphoric acid at a level greater than 1 weight %, greater than 0.5 weight %, greater than 0.4 weight %, 0.3 weight %, or greater than 0.2 weight %, as measured by 31P NMR. In some embodiments, crystalline psilocybin Polymorph A′ has a chemical assay of at least 95 weight %, at least 96 weight %, or at least 98 weight %.

Illustrative XRPD diffractograms for high purity crystalline psilocybin, Polymorph A or Polymorph A′ are provided in FIGS. 2A and 2B. Illustrative DSC thermographs for high purity crystalline psilocybin, Polymorph A or Polymorph A′ are provided in FIGS. 2A and 2B.

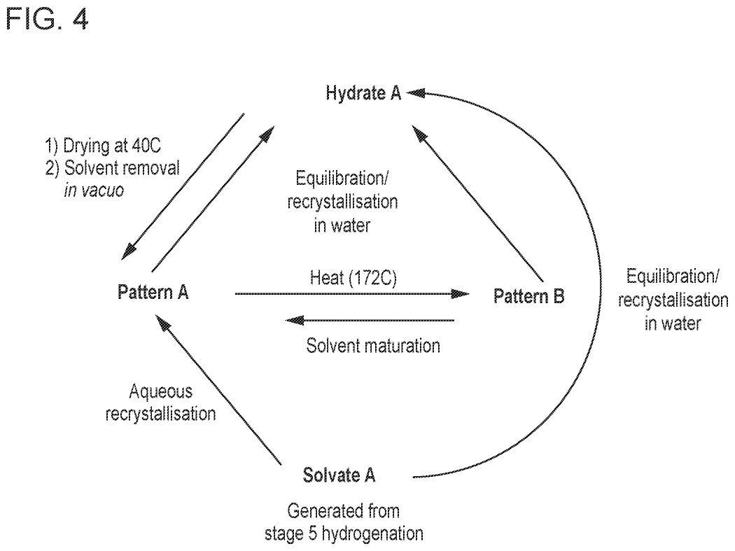

Polymorph A (including its isostructural variant Polymorph A′) (FIGS. 2A and 2B) differs from Polymorph B (FIG. 2C), the Hydrate A (FIG. 2D) and the ethanol solvate (FIG. 2E: Solvate A), and the relationship between some of the different forms is illustrated in FIG. 4.

In some embodiments, the crystalline psilocybin Polymorph A or Polymorph A′ is a white to off white solid, and/or has a chemical purity of greater than 97%, 98%, or 99% by HPLC. In some embodiments, crystalline psilocybin Polymorph A or Polymorph A′ has no single impurity of greater than 1%, greater than 0.5%, greater than 0.4%, greater than 0.3%, or greater than 0.2%, e.g., the impurity phosphoric acid as measured by 31P NMR, or the impurity psilocin measured by HPLC. In some embodiments, crystalline psilocybin Polymorph A or Polymorph A′ has a chemical purity of greater than 97 area %, greater than 98 area %, or greater than 99 area % by HPLC. In some embodiments, crystalline psilocybin Polymorph A or Polymorph A′ has no single impurity greater than 1 area %, greater than 0.5 area %, greater than 0.4%, greater than 0.3%, or greater than 0.2% as measured by HPLC. In some embodiments, crystalline psilocybin Polymorph A or Polymorph A′ does not contain psilocin at a level greater than 1 area %, greater than 0.5 area %, greater than 0.4%, greater than 0.3%, or greater than 0.2% as measured by HPLC. In some embodiments, crystalline psilocybin Polymorph A or Polymorph A′ does not contain phosphoric acid at a level greater than 1 weight %, greater than 0.5 weight %, greater than 0.4 weight %, 0.3 weight %, or greater than 0.2 weight %, as measured by 31P NMR. In some embodiments, crystalline psilocybin Polymorph A or Polymorph A′ has a chemical assay of at least 95 weight %, at least 96 weight %, or at least 98 weight %.

The heating of Polymorph A or A′ results in an endothermic event having an onset temperature of circa 150° C. corresponding to solid-solid transition of Polymorph A or Polymorph A′ to Polymorph B. Continued heating of the resulting solid, i.e., Polymorph B, results in a second endothermic event corresponding to a melting point having an onset temperature of between 205 and 220° C. (see FIGS. 3A and 3B).

### Hydrate A

In some embodiments, the disclosure provides a crystalline form of psilocybin, Hydrate A. In some embodiments, crystalline psilocybin Hydrate A exhibits peaks in an XRPD diffractogram at 8.9, 12.6 and 13.8 °2θ±0.1 °2θ. In some embodiments, crystalline psilocybin Hydrate A further exhibits at least 1, 2, 3, 4, or 5 further peaks at 6.5, 12.2, 19.4, 20.4 or 20.8 °2θ±0.1 °2θ. An illustrative XRPD diffractogram is provided as FIG. 2D. In some embodiments, crystalline psilocybin Hydrate A further exhibits an endothermic event in a DSC thermogram having a first onset temperature of between 90° C. and 100° C., a second onset temperature of between 100° C. and 120° C. and a third onset temperature of between 210° C. and 220° C. An illustrative DSC thermogram is provided as FIG. 2D.

In some embodiments, psilocybin Hydrate A exhibits an XRPD diffractogram comprising at least 3, 4, 5, 6, 7, 8, 9, or 10 peaks listed in Table C or equivalent peaks within about ±0.1 °2θ.

TABLE C
XRPD peak positions for Hydrate A

| | Relative |
|---|---|
| Position | Intensity |
| [°2Th.] | [%] |
| 5.6 | 14.40 |
| 6.5 | 18.84 |
| 8.9 | 100.00 |
| 12.2 | 11.51 |
| 12.6 | 18.65 |
| 13.8 | 44.22 |
| 16.2 | 21.22 |
| 18.9 | 6.62 |
| 19.4 | 38.68 |
| 20.4 | 21.32 |
| 20.8 | 19.73 |
| 21.5 | 20.75 |
| 22.3 | 12.80 |
| 22.5 | 19.38 |
| 23.1 | 47.53 |
| 23.5 | 25.79 |
| 24.3 | 5.62 |
| 24.8 | 14.62 |
| 25.4 | 5.27 |
| 26.9 | 6.53 |
| 27.9 | 7.82 |
| 28.4 | 5.78 |
| 29.0 | 5.09 |
| 29.7 | 4.83 |
| 32.1 | 8.27 |
| 32.8 | 4.81 |
| 33.4 | 3.74 |
| 34.2 | 5.96 |

In some embodiments, crystalline psilocybin Hydrate A exhibits XRPD diffractogram peaks at 8.9, 12.6 and 13.8 °2θ±0.1 °2θ. In some embodiments, crystalline psilocybin Hydrate A exhibits at least one peak appearing at 6.5, 12.2, 19.4, 20.4 or 20.8 °2θ±0.1 °2θ. In some embodiments, crystalline psilocybin Hydrate A exhibits at least two peaks appearing at 6.5, 12.2, 19.4, 20.4 or 20.8 °2θ±0.1 °2θ. In some embodiments, crystalline psilocybin Hydrate A exhibits an XRPD diffractogram substantially the same as the XRPD diffractogram shown in FIG. 2D.

In certain embodiments, crystalline psilocybin Hydrate A is characterized by an endothermic event in a DSC thermogram having a first onset temperature of between 85° C. and 105° C., such as between 90° C. and 100° C. and most preferably at about 96° C., a second onset temperature of between 100° C. and 120° C. such as between 105° C. and 115° C., and most preferably at about 109° C. and a third onset temperature of between 205 and 220° C., such as between 21° and 220° C., such as between 21° and 218° C., or such as between 21° and 216° C., or about 216° C. In some embodiments, crystalline psilocybin Hydrate A exhibits an endothermic event in a DSC thermogram having an onset temperature of between about 205 and about 220° C., between about 210 and about 220° C., between about 210 and about 218° C., or between about 210 and about 216° C. In some embodiments, crystalline psilocybin Hydrate A exhibits an endothermic event in the DSC thermogram having an onset temperature of between about 85 and about 105° C., or between about 90 and about 100° C. In some embodiments, crystalline psilocybin Hydrate A exhibits an endothermic event having an onset temperature of between about 205 and about 220° C., between about 210 and about 220° C., between about 210 and about 218° C., or between about 210 and about 216° C., and an endothermic event having an onset temperature of between about 85 and about 105° C. or between about 90 and about 100° C., in a DSC thermogram. In some embodiments, crystalline psilocybin Hydrate A exhibits a DSC thermogram substantially the same as the DSC thermogram in FIG. 3D.

In some embodiments, crystalline psilocybin Hydrate A exhibits a water content of between about 10 and about 18%, between about 12 and about 16%, or about 13%. Methods to determine the water content of a crystalline compound are known, for example Karl Fischer Titration. In some embodiments, crystalline psilocybin Hydrate A exhibits a weight loss in the TGA thermogram of between about 10 and about 18%, between about 12 and about 16%, or about 13%, between ambient temperature, about 25° C., and 120° C.

In some embodiments, crystalline psilocybin Hydrate A is chemically pure, for example the psilocybin has a chemical purity of greater than 97%, 98%, or 99% by HPLC. In some embodiments, crystalline psilocybin Hydrate A has no single impurity of greater than 1%, greater than 0.5%, greater than 0.4%, greater than 0.3%, or greater than 0.2% e.g., the impurity phosphoric acid as measured by 31P NMR, or the impurity psilocin measured by HPLC. In some embodiments, crystalline psilocybin Hydrate A has a chemical purity of greater than 97 area %, greater than 98 area %, or greater than 99 area % by HPLC. In some embodiments, crystalline psilocybin Hydrate A has no single impurity greater than 1 area %, greater than 0.5 area %, greater than 0.4%, greater than 0.3%, or greater than 0.2% as measured by HPLC. In some embodiments, crystalline psilocybin Hydrate A does not contain psilocin at a level greater than 1 area %, greater than 0.5 area %, greater than 0.4%, greater than 0.3%, or greater than 0.2% as measured by HPLC. In some embodiments, crystalline psilocybin Hydrate A does not contain phosphoric acid at a level greater than 1 weight %, greater than 0.5 weight %, greater than 0.4 weight %, 0.3 weight %, or greater than 0.2 weight %, as measured by 31P NMR. In some embodiments, crystalline psilocybin Hydrate A has a chemical assay of at least 95 weight %, at least 96 weight %, or at least 98 weight %.

In some embodiments, crystalline psilocybin Hydrate A is a highly pure crystalline form of Hydrate A. In some embodiments, the crystalline psilocybin comprises at least 90%, at least 95%, at least 99%, or at least 99.5% by weight of Hydrate A.

### Polymorph B

In some embodiments, the disclosure provides a crystalline form of psilocybin, Polymorph B. In some embodiments, crystalline psilocybin Polymorph B exhibits peaks in an XRPD diffractogram at 11.1, 11.8 and 14.3 °2θ±0.1 °2θ. In some embodiments, crystalline psilocybin Polymorph B exhibits at least 1, 2, 3, 4 or 5 peaks in an XRPD diffractogram at 14.9, 15.4, 19.3, 20.0 or 20.6 °2 θ±0.1 °2θ. An illustrative XRPD diffractogram of crystalline psilocybin Polymorph B is provided as FIG. 2C. In some embodiments, crystalline psilocybin Polymorph B exhibits a single endothermic event in a DSC thermogram having an onset temperature of between about 205 and about 220° C. An illustrative DSC thermogram of crystalline psilocybin Polymorph B is provided as FIG. 3C.

In some embodiments, psilocybin Polymorph B exhibits an XRPD diffractogram comprising at least 3, 4, 5, 6, 7, 8, 9, or 10 peaks listed in Table D or equivalent peaks within about ±0.1 °2θ.

TABLE D
XRPD peak positions for Polymorph B

| | Relative |
|---|---|
| Position | Intensity |
| [°2Th.] | [%] |
| 5.5 | 21.33 |
| 11.1 | 36.91 |
| 11.8 | 100.00 |
| 12.5 | 12.73 |
| 14.3 | 70.23 |
| 14.9 | 50.01 |
| 15.4 | 23.67 |
| 17.1 | 51.58 |
| 17.4 | 91.25 |
| 18.0 | 12.61 |
| 19.3 | 39.33 |
| 20.0 | 76.61 |
| 20.6 | 50.26 |
| 21.5 | 20.77 |
| 22.3 | 40.19 |
| 23.9 | 13.32 |
| 24.3 | 16.03 |
| 25.3 | 32.94 |
| 28.3 | 7.60 |
| 28.9 | 17.89 |
| 29.3 | 8.96 |
| 31.3 | 6.57 |
| 32.2 | 6.90 |
| 33.8 | 2.37 |

In some embodiments, crystalline psilocybin Polymorph B exhibits XRPD diffractogram peaks at 11.1, 11.8 and 14.3 °2θ±0.1 °2θ. In some embodiments, crystalline psilocybin Polymorph B exhibits at least one peak at 14.9, 15.4, 19.3, 20.0 or 20.6 °2θ±0.1 °2θ. In some embodiments, crystalline psilocybin Polymorph B exhibits at least two peaks appearing at 14.9, 15.4, 19.3, 20.0 or 20.6 °2θ±0.1 °2θ. In some embodiments, crystalline psilocybin Polymorph B exhibits an XRPD diffractogram substantially the same as the XRPD diffractogram shown in FIG. 2C.

In some embodiments, crystalline psilocybin Polymorph B is characterized by a single endothermic event in a DSC thermogram having an onset temperature of between about 205 and about 220° C., between about 210 and about 220° C., between about 210 and about 218° C., or between about 210 and about 216° C. In some embodiments, crystalline psilocybin Polymorph B exhibits a DSC thermogram substantially the same as the DSC thermogram in FIG. 3C.

In some embodiments, crystalline psilocybin Polymorph B exhibits a water content of <0.5% w/w, <0.4% w/w, <0.3% w/w, <0.2% w/w, or <0.1% w/w. Methods to determine the water content of a crystalline compound are known, for example Karl Fischer Titration. In some embodiments, crystalline psilocybin Polymorph B exhibits <0.5% w/w, <0.4% w/w, <0.3% w/w, <0.2% w/w, or <0.1% w/w loss in the TGA thermogram between ambient temperature, about 25° C., and 200° C. In some embodiments, crystalline psilocybin Polymorph B exhibits a loss of less than 2% by weight, less than 1% by weight, or less than 0.5% by weight in a loss on drying test. In some embodiments, the loss on drying test is performed at 70° C.

In some embodiments, crystalline psilocybin Polymorph B is a highly pure crystalline form of Polymorph B, for example, psilocybin comprises at least 90%, at least 95%, at least 99%, or at least 99.5% by weight of Polymorph B.

In some embodiments, crystalline psilocybin Polymorph B is chemically pure, for example the psilocybin has a chemical purity of greater than 97%, 98%, or 99% by HPLC. In some embodiments, crystalline psilocybin Polymorph B has no single impurity of greater than 1%, greater than 0.5%, greater than 0.4%, greater than 0.3%, or greater than 0.2% e.g., the impurity phosphoric acid as measured by 31P NMR, or the impurity psilocin measured by HPLC. In some embodiments, crystalline psilocybin Polymorph B has a chemical purity of greater than 97 area %, greater than 98 area %, or greater than 99 area % by HPLC. In some embodiments, crystalline psilocybin Polymorph B has no single impurity greater than 1 area %, greater than 0.5 area %, greater than 0.4%, greater than 0.3%, or greater than 0.2% as measured by HPLC. In some embodiments, crystalline psilocybin Polymorph B does not contain psilocin at a level greater than 1 area %, greater than 0.5 area %, greater than 0.4%, greater than 0.3%, or greater than 0.2% as measured by HPLC. In some embodiments, crystalline psilocybin Polymorph B does not contain phosphoric acid at a level greater than 1 weight %, greater than 0.5 weight %, greater than 0.4 weight %, 0.3 weight %, or greater than 0.2 weight %, as measured by 31P NMR. In some embodiments, crystalline psilocybin Polymorph B has a chemical assay of at least 95 weight %, at least 96 weight %, or at least 98 weight %.

In some embodiments, the psilocybin of the disclosure in the form Polymorph A or A′ has the general properties illustrated in Table D.

TABLE D

| | |
|---|---|
| Appearance: | White to off-white solid |
| Major endothermic event in | 210-215° C. |
| DSC (onset temperature) | |
| (corresponding to a melt): | |
| Hygroscopicity: | Psilocybin forms Hydrate A at high humidity |
| | and when added to water but the water of |
| | hydration is lost rapidly on drying. The |
| | anhydrous form is therefore being |
| | developed. |
| Crystalline form: | Anhydrous Polymorph A and/ or A′ |
| pKa (calculated): | 1.74, 6.71, 9.75 |
| Solubility | approx. 15 mg/ml in Water |

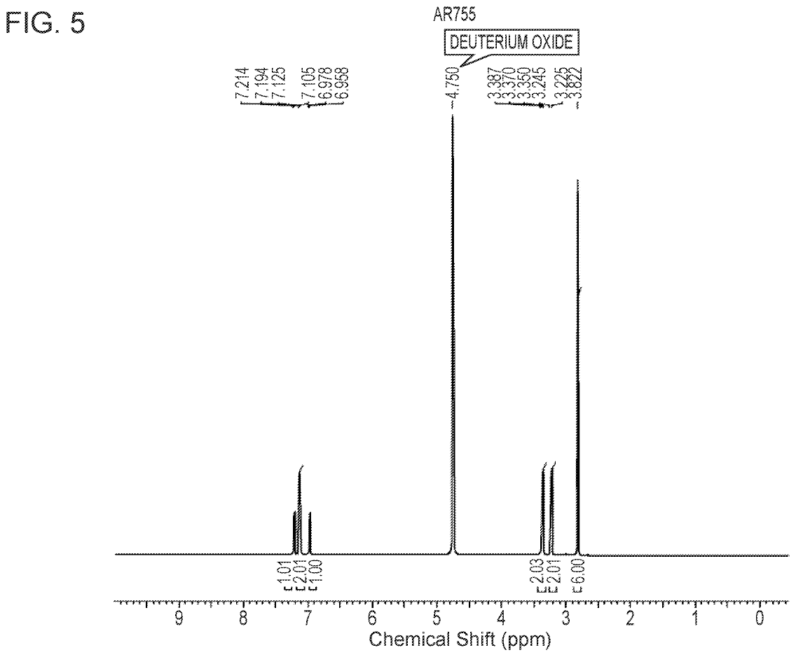

In some embodiments, the psilocybin conforms to the spectra as set out in Table E and illustrated in the spectra of FIGS. 5-8.

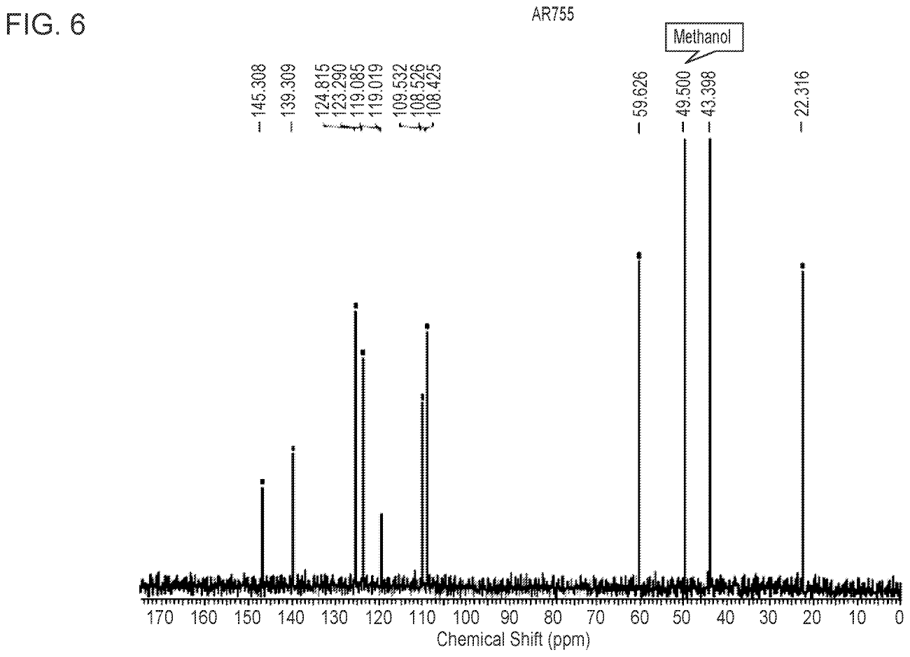

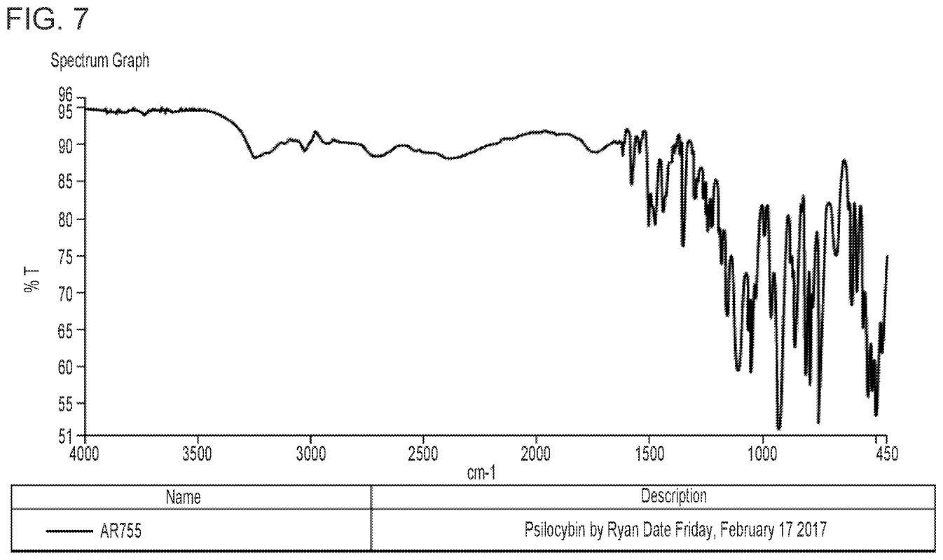

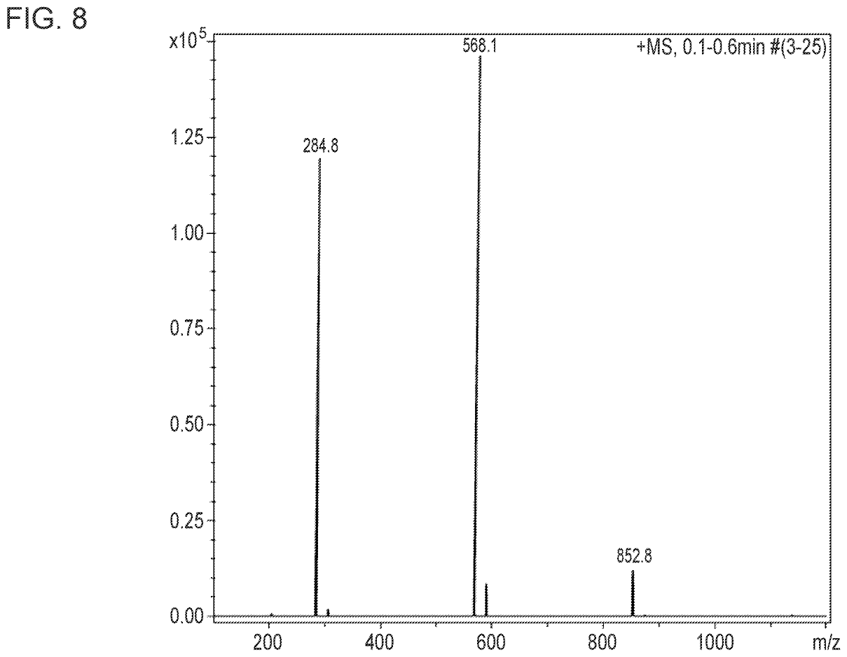

TABLE E

| Technique | Conclusions |
|---|---|
| Proton (¹H) and | Assignment of the proton (FIG. 5) and |
| Carbon (¹³C) NMR | carbon spectra (FIG. 6) are concordant with |
| | Psilocybin. |
| FT-Infrared | Assignment of the FT-IR spectrum (FIG. 7) |
| Spectroscopy (FT-IR) | is concordant with Psilocybin. |
| Mass Spectroscopy | Assignment of the mass spectrum (FIG. 8) |
| (MS) | is concordant with Psilocybin. |

Alternatively, and independently, the crystalline psilocybin may take the form of Hydrate A or Polymorph B.

In some embodiments, the disclosure provides the crystalline psilocybin in the form Polymorph A or Polymorph A′ for use in medicine. In some embodiments, the disclosure provides crystalline psilocybin Polymorph A for use in medicine. In some embodiments, the disclosure provides crystalline psilocybin Polymorph A′ for use in medicine. In some embodiments, the disclosure provides a high purity crystalline psilocybin Polymorph A for use in medicine. In some embodiments, the disclosure provides a high purity crystalline psilocybin Polymorph A′ for use in medicine. Alternatively, and independently, the crystalline psilocybin may take the form of Hydrate A or Polymorph B.

In some embodiments, the disclosure provides crystalline psilocybin in the form Polymorph A or Polymorph A′ for use in treating a subject in need thereof. Alternatively, and independently, the crystalline psilocybin may take the form of Hydrate A or Polymorph B.

In some embodiments, the disclosure provides crystalline psilocybin, Polymorph A or Polymorph A′, for use in treating a subject in need thereof. In some embodiments, the disclosure provides crystalline psilocybin, Polymorph A or Polymorph A′, for use in treating a subject in need thereof. In some embodiments, the disclosure provides crystalline psilocybin Polymorph A for use in treating a subject in need thereof. In some embodiments, the disclosure provides crystalline psilocybin Polymorph A′ for use in treating a subject in need thereof. In some embodiments, the disclosure provides a high purity crystalline psilocybin Polymorph A for use in treating a subject in need thereof. In some embodiments, the disclosure provides a high purity crystalline psilocybin Polymorph A′ for use in treating a subject in need thereof.

### Pharmaceutical Compositions and Formulations

In some embodiments, the disclosure provides a pharmaceutical composition comprising crystalline psilocybin and one or more pharmaceutically acceptable carriers or excipients.

In some embodiments, the disclosure provides a pharmaceutical formulation comprising high purity psilocybin and one or more pharmaceutically acceptable carriers or excipients. In some embodiments, the disclosure provides a pharmaceutical formulation comprising crystalline psilocybin Polymorph A and one or more pharmaceutically acceptable carriers or excipients. In some embodiments, the disclosure provides a pharmaceutical formulation comprising crystalline psilocybin Polymorph A′ and one or more pharmaceutically carriers or excipients. In some embodiments, the disclosure provides a pharmaceutical formulation comprising high purity crystalline psilocybin, Polymorph A or Polymorph A′, and one or more pharmaceutically acceptable carriers or excipients. In some embodiments, the disclosure provides a pharmaceutical formulation comprising high purity crystalline psilocybin Polymorph A and one or more pharmaceutically acceptable carriers or excipients. In some embodiments, the disclosure provides a pharmaceutical formulation comprising high purity crystalline psilocybin Polymorph A′ and one or more pharmaceutically acceptable carriers or excipients.

In some embodiments, a pharmaceutical composition or formulation described herein comprises psilocybin, pergelatanized starch, and sodium stearyl fumarate. In some embodiments, the pharmaceutical composition or formulation comprises about 1%-10%, by weight, psilocybin. In some embodiments, the pharmaceutical composition or formulation comprises about 85-99%, by weight, pregelatinized starch. In some embodiments, the pharmaceutical composition or formulation comprises about 0.5%-2%, by weight, sodium stearyl fumarate.

In embodiments, a pharmaceutical composition or formulation described herein comprises (a) about 1%-10%, by weight, psilocybin; (b) about 85-99%, by weight, pregelatinized starch; and (c) about 0.5%-2%, by weight, sodium stearyl fumarate. In some embodiments, the pharmaceutical composition or formulation is one or more pharmaceutical compositions or formulations described in WIPO Patent Appln. Pub. No. 2022/207746 and 2019/073379, the entire contents of which are hereby incorporated by reference.

Preferred pharmaceutical excipients for an oral formulation include: diluents, such as microcrystalline cellulose, starch, mannitol, calcium hydrogen phosphate anhydrous or co-mixtures of silicon dioxide, calcium carbonate, microcrystalline cellulose and talc; disintegrants, such as sodium starch glycolate or croscarmellose sodium; binders, such as povidone, co-povidone or hydroxyl propyl cellulose; lubricants, such as magnesium stearate or sodium stearyl fumurate; glidants, such as colloidal silicon dioxide; and film coats, such as Opadry II white or PVA based brown Opadry II.

In some embodiments, the oral dosage form also comprises a disintegrant, such as, but not limited to: starch glycolate, croscarmellose sodium, and/or mixtures thereof. In some embodiments, the oral dosage form comprises 3% or less by wt disintegrant, less than 3% by wt disintegrant and greater than 0.001% by wt disintegrant, about 2.5% by wt or less disintegrant; 2% by wt or less disintegrant; 1.5% by wt or less disintegrant; 1% by wt or less disintegrant; 0.7% by wt or less disintegrant; 0.5% by wt or less disintegrant, or 0.3% by wt or less disintegrant.

In some embodiments, the disintegrant is sodium starch glycolate. In some embodiments, the sodium starch glycolate is present at less than 3% wt. In Other embodiments, the sodium starch glycolate is present at about 2% by wt or less, about 2% by wt; about 1% by wt or less, about 1% by wt; about 0.7% by wt or less, about 0.7% by wt; about 0.5% by wt or less, or about 0.5% by wt. In still other embodiments, the sodium starch glycolate is present at about 0.5% to 1% by wt.

In some embodiments, the oral dosage form comprises 5 mg of psilocybin and SMCC 50 and SMCC 90, wherein the ratio of SMCC 50 to SMCC 90 is 1:6.4 and sodium starch glycolate at about 1%. In some embodiments, the oral dosage form comprises 5 mg of psilocybin and SMCC 50 and SMCC 90, wherein the ratio of SMCC 50 to SMCC 90 is 1:6.4 and sodium starch glycolate at about 0.5% to 1.0%. In some embodiments, the oral dosage form comprises 5 mg of psilocybin and SMCC 50 and SMCC 90, wherein the ratio of SMCC 50 to SMCC 90 is 1:6.4 and sodium starch glycolate at about 0.5%.

In some embodiments, the oral dosage form comprises 10 mg of psilocybin and SMCC 50 and SMCC 90, wherein the ratio of SMCC 50 to SMCC 90 is 1:6.4 and sodium starch glycolate at about 1%. In some embodiments, the oral dosage form comprises 10 mg of psilocybin and SMCC 50 and SMCC 90, wherein the ratio of SMCC 50 to SMCC 90 is 1:6.4 and sodium starch glycolate at about 0.5% to 1.0%. In some embodiments, the oral dosage form comprises 10 mg of psilocybin and SMCC 50 and SMCC 90, wherein the ratio of SMCC 50 to SMCC 90 is 1:6.4 and sodium starch glycolate at about 0.5%.

In some embodiments, the oral dosage form comprises 25 mg of psilocybin and SMCC 50 and SMCC 90, wherein the ratio of SMCC 50 to SMCC 90 is 1:6.4 and sodium starch glycolate at about 1%. In some embodiments, the oral dosage form comprises 25 mg of psilocybin and SMCC 50 and SMCC 90, wherein the ratio of SMCC 50 to SMCC 90 is 1:6.4 and sodium starch glycolate at about 0.5% to 1.0%. In some embodiments, the oral dosage form comprises 25 mg of psilocybin and SMCC 50 and SMCC 90, wherein the ratio of SMCC 50 to SMCC 90 is 1:6.4 and sodium starch glycolate at about 0.5%.

In some embodiments there is provided the crystalline psilocybin in the form Polymorph A or Polymorph A′ for use in medicine. In some embodiments, there is provided crystalline psilocybin Polymorph A for use in medicine. In some embodiments, there is provided crystalline psilocybin Polymorph A′ for use in medicine. In some embodiments, there is provided a high purity crystalline psilocybin Polymorph A for use in medicine. In some embodiments, there is provided a high purity crystalline psilocybin Polymorph A′ for use in medicine.

Alternatively, and independently, the crystalline psilocybin may take the form of Hydrate A or Polymorph B.

In some embodiments, there is provided crystalline psilocybin, particularly but not essentially in the form Polymorph A or Polymorph A′ for use in treating central nervous disorders.

Alternatively, and independently, the crystalline psilocybin may take the form of Hydrate A or Polymorph B.

In some embodiments, the pharmaceutical formulation is a parenteral dosage form. In some embodiments, the pharmaceutical formulation is an oral dosage form. In some embodiments, the pharmaceutical composition comprises a tablet. In some embodiments, the pharmaceutical composition comprises a capsule. In some embodiments, the pharmaceutical composition comprises a dry powder. In some embodiments, the pharmaceutical composition comprises a solution. In some embodiments, more than one dosage form is administered to the subject at substantially the same time. In some embodiments, the subject may be administered the entire therapeutic dose in one tablet or capsule. In some embodiments, the therapeutic dose may be split among multiple tablets or capsules. For example, for a dose of 25 mg, the subject may be administered 5 tablets or capsules each comprising 25 mg of psilocybin. Alternatively, for a dose of 10 mg, the subject may be administered 2 tablets or capsules each comprising 5 mg of psilocybin.

In some embodiments, the oral dosage form comprises a functional filler. The functional filler may be a silicified filler, such as, but not limited to silicified microcrystalline cellulose (SMCC). In some embodiments, the oral dosage form comprises high compactability grades of SMCC with a particle size range of from about 45 to 150 microns. A mixture of two functional fillers having different particle size ranges may be used with the weight percentages of the two favoring the larger sized particles.

In some embodiments, the silicified microcrystalline filler may comprise a first filler, having a particle size range of from about 45 to 80 microns in an amount of up to 30%, up to 20%, up to 15%, or less by weight of filler, and a second filler, having a particle size range of from about 90 to 150 microns, in an amount of up to 70%, up to 80%, up to 85%, or more, by weight of filler.

In some embodiments, the oral dosage form may comprise silicified microcrystalline cellulose with a particle size range of from about 45 to 80 microns (SMCC 50), such as Prosolv 50; silicified microcrystalline cellulose with a particle size range of from about 90 to 150 microns (SMCC 90), such as Prosolv 90; or mixtures thereof. In other embodiments, the oral dosage form may comprise SMCC 50 and SMCC 90. In other embodiments, the oral dosage form may comprise SMCC 50 and SMCC 90, wherein the ratio of SMCC 50 to SMCC 90 is 1:5 to 1:8 wt %. In still other embodiments the ratio of SMCC 50 to SMCC 90 is 1:5-1:7; 1:6-1:7; 1:6-1:8; or 1.7-1.8. In still other embodiments the ratio of SMCC 50 to SMCC 90 is 1:6; 1:6.1; 1:6.2; 1:6.3; 1:6.4; 1:6.5; 1:6.6; 1.6.7; 1:6.8; 1.6.9; or 1:7. The formulation may further comprise or consist essentially of a disintegrant, including without limitation sodium starch glycolate; a glidant, including without limitation colloidal silicon dioxide; and a lubricant, including without limitation sodium stearyl fumarate.

In some embodiments, the oral dosage form may comprise a disintegrant such as sodium starch glycolate, at less than 3% (by wt), less than 2%, or 1% or less.

In some embodiments, the oral dosage form comprises 5 mg of psilocybin and SMCC 50 and SMCC 90, wherein the ratio of SMCC 50 to SMCC 90 is 1:6.4 and sodium starch glycolate at about 1%. In some embodiments, the oral dosage form comprises 5 mg of psilocybin and SMCC 50 and SMCC 90, wherein the ratio of SMCC 50 to SMCC 90 is 1:6.4 and sodium starch glycolate at about 0.5% to 1.0%. In some embodiments, the oral dosage form comprises 5 mg of psilocybin and SMCC 50 and SMCC 90, wherein the ratio of SMCC 50 to SMCC 90 is 1:6.4 and sodium starch glycolate at about 0.5%.

In some embodiments, the oral dosage form comprises 10 mg of psilocybin and SMCC 50 and SMCC 90, wherein the ratio of SMCC 50 to SMCC 90 is 1:6.4 and sodium starch glycolate at about 1%. In some embodiments, the oral dosage form comprises 10 mg of psilocybin and SMCC 50 and SMCC 90, wherein the ratio of SMCC 50 to SMCC 90 is 1:6.4 and sodium starch glycolate at about 0.5% to 1.0%. In some embodiments, the oral dosage form comprises 10 mg of psilocybin and SMCC 50 and SMCC 90, wherein the ratio of SMCC 50 to SMCC 90 is 1:6.4 and sodium starch glycolate at about 0.5%.

In some embodiments, the oral dosage form comprises 25 mg of psilocybin and SMCC 50 and SMCC 90, wherein the ratio of SMCC 50 to SMCC 90 is 1:6.4 and sodium starch glycolate at about 1%. In some embodiments, the oral dosage form comprises 25 mg of psilocybin and SMCC 50 and SMCC 90, wherein the ratio of SMCC 50 to SMCC 90 is 1:6.4 and sodium starch glycolate at about 0.5% to 1.0%. In some embodiments, the oral dosage form comprises 25 mg of psilocybin and SMCC 50 and SMCC 90, wherein the ratio of SMCC 50 to SMCC 90 is 1:6.4 and sodium starch glycolate at about 0.5%.

In some embodiments, the oral dosage form comprises 5 mg of crystalline psilocybin in the form of Polymorph A, 12.5 mg of SMCC 50, 79.5 mg of SMCC 90, 1 mg sodium starch glycolate, 1 mg colloidal silicon dioxide and 1 mg sodium stearyl fumarate. In some embodiments, the tablet or capsule comprises 5 mg of crystalline psilocybin in the form of Polymorph A, 12.5 mg of SMCC 50, 79.5 mg of SMCC 90, 1 mg sodium starch glycolate, 1 mg colloidal silicon dioxide and 1 mg sodium stearyl fumarate.

In some embodiments, the oral dosage form comprises 1 mg of crystalline psilocybin in the form of Polymorph A, 20.5 mg of SMCC 50, 75.5 mg of SMCC 90, 1 mg sodium starch glycolate, 1 mg colloidal silicon dioxide, and 1 mg sodium stearyl fumarate. In some embodiments, the tablet or capsule comprises 1 mg of crystalline psilocybin in the form of Polymorph A, 20.5 mg of SMCC 50, 75.5 mg of SMCC 90, 1 mg sodium starch glycolate, 1 mg colloidal silicon dioxide, and 1 mg sodium stearyl fumarate.

In some embodiments, the tablet or capsule comprises one or more excipients. Non-limiting exemplary excipients include microcrystalline cellulose and starch, including without limitation silicified microcrystalline cellulose.

It should be noted that the formulations may comprise psilocybin in any form, not only the polymorphic forms disclosed herein.

As used herein, oral doses of psilocybin are classified follows: “very low doses” (about 0.045 mg/kg or less); “low doses” (between about 0.115 and about 0.125 mg/kg), “medium doses” (between about 0.115 to about 0.260 mg/kg), and “high doses” (about 0.315 mg/kg or more). See Studerus et al (2011) J Psychopharmacol 25 (11) 1434-1452.

In some embodiments, the formulated dose of psilocybin comprises from about 0.01 mg/kg to about 1 mg/kg. In some embodiments, a human dose (for an adult weighing 60-80 kg) comprises between about 0.60 mg and about 80 mg.

In some embodiments, a formulated dose comprises between about 2 and about 50 mg of crystalline psilocybin. In some embodiments, a formulated dose comprises between 2 and 40 mg, between 2 and 10 mg, between 5 and 30 mg, between 5 and 15 mg, or between 20 and 30 mg of crystalline psilocybin. In some embodiments, a formulated dose comprises about 1 mg, about 5 mg, about 10 mg, or about 25 mg of crystalline psilocybin.

In some embodiments, a formulated dose comprises between about 2 and about 50 mg of crystalline psilocybin Polymorph A or Polymorph A′ or a mixture thereof. In some embodiments, a formulated dose comprises between 2 and 40 mg, between 2 and 10 mg, between 5 and 30 mg, between 5 and 15 mg, or between 20 and 30 mg of crystalline psilocybin Polymorph A or Polymorph A′ or a mixture thereof. In some embodiments, a formulated dose comprises about 1 mg, about 5 mg, about 10 mg, or about 25 mg of crystalline psilocybin Polymorph A or Polymorph A′ or a mixture thereof. In some embodiments, a formulated dose comprises about 5 mg of crystalline psilocybin Polymorph A or Polymorph A′ or a mixture thereof.

In some embodiments, a formulated dose comprises between about 2 and about 50 mg of crystalline psilocybin Polymorph A. In some embodiments, a formulated dose comprises between 2 and 40 mg, between 2 and 10 mg, between 5 and 30 mg, between 5 and 15 mg, or between 20 and 30 mg of crystalline psilocybin Polymorph A. In some embodiments, a formulated dose comprises about 1 mg, about 5 mg, about 10 mg, or about 25 mg of crystalline psilocybin Polymorph A.

In some embodiments, a formulated dose comprises between about 2 and about 50 mg of crystalline psilocybin Polymorph A′. In some embodiments, a formulated dose comprises between 2 and 40 mg, between 2 and 10 mg, between 5 and 30 mg, between 5 and 15 mg, or between 20 and 30 mg of crystalline psilocybin Polymorph A′. In some embodiments, a formulated dose comprises about 1 mg, about 5 mg, about 10 mg, or about 25 mg of crystalline psilocybin Polymorph A′.

In some embodiments, a formulated dose comprises between about 2 and about 50 mg of crystalline psilocybin Polymorph B. In some embodiments, a formulated dose comprises between 2 and 40 mg, between 2 and 10 mg, between 5 and 30 mg, between 5 and 15 mg, or between 20 and 30 mg of crystalline psilocybin Polymorph B. In some embodiments, a formulated dose comprises about 1 mg, about 5 mg, about 10 mg, or about 25 mg of crystalline psilocybin Polymorph B.

In some embodiments, a formulated dose comprises between about 2 and about 50 mg of crystalline psilocybin Hydrate A. In some embodiments, a formulated dose comprises between 2 and 40 mg, between 2 and 10 mg, between 5 and 30 mg, between 5 and 15 mg, or between 20 and 30 mg of crystalline psilocybin Hydrate A. In some embodiments, a formulated dose comprises about 1 mg, about 5 mg, about 10 mg, or about 25 mg of crystalline psilocybin Hydrate A.

### Dosing

In some embodiments, a therapeutically effective dose of psilocybin is administered to the subject. In some embodiments, each dose of psilocybin administered to the subject is a therapeutically effective dose.

In some embodiments, a dose of psilocybin may be in the range of about 1 mg to about 100 mg. For example, the dose may be about 1 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, or about 100 mg. In some embodiments, the dose of psilocybin is between about 0.1 mg to about 100 mg, about 1 mg to about 50 mg, or about 5 mg to about 30 mg. In some embodiments, the dose of psilocybin is about 1 mg, about 10 mg, or about 25 mg. In some embodiments, the dose of psilocybin is in the range of about 0.001 mg to about 1 mg. In some embodiments, the dose of psilocybin is in the rage of about 100 mg to about 250 mg. In some embodiments, the dose of psilocybin is about 25 mg. In some embodiments, the psilocybin is in the form of polymorph A.

In some embodiments, an adult oral dose comprises about 1 mg to about 40 mg, about 2 to about 30 mg, or about 15 to about 30 mg of crystalline psilocybin, for example about 1 mg, about 5 mg, about 10 mg, or about 25 mg of crystalline psilocybin. In some embodiments, an adult oral dose comprises about 25 mg of crystalline psilocybin. In some embodiments, the crystalline psilocybin is in the form of polymorph A.

In some embodiments, a “micro-dose” of psilocybin is administered to a subject. A micro-dose may comprise, for example, about 0.05 mg to about 2.5 mg of crystalline psilocybin, such as about 1.0 mg. In the case of micro-dosing the regime may comprise a regular, continuous regime of, for example, daily administration, every other day administration, or weekly, administration. Such dosing may be absent of psychological support.

In some embodiments, one dose of psilocybin is administered to the subject. In some embodiments, multiple doses of psilocybin are administered to the subject. For example, at least 2, at least 3, at least 4, at least 5, at least 6, at least 7, at least 8, at least 9, at least 10, at least 15, at least 20, at least 25, at least 30, or at least 50 doses of psilocybin may be administered to the subject. In some embodiments, the same dose of psilocybin is administered to a subject during each administration. In some embodiments, a different dose of psilocybin is administered to a subject during each administration. In some embodiments, the dose of psilocybin administered to the subject is increased over time. In some embodiments, the dose of psilocybin administered to the subject is decreased over time.

In some embodiments, the psilocybin is administered at therapeutically effective intervals. In some embodiments, a therapeutically effective interval may be about 2 weeks, about 3 weeks, about 4 weeks, about 5 weeks, about 6 weeks, about 7 weeks, about 8 weeks, about 9 weeks, about 10 weeks, about 11 weeks, or about 12 weeks. In some embodiments, a therapeutically effective interval may be about 1 month, about 3 months, about 6 months, or about 12 months. In some embodiments, the psilocybin is administered once per day. In some embodiments, the psilocybin is administered at least once per week or at least twice per week. In some embodiments, the psilocybin is administered at least once per month or at least twice per month. In some embodiments, the psilocybin is administered at least once every three months, at least once every six months, or at least once every 12 months.

In some embodiments, a first dose and a second dose of psilocybin are administered to the subject. In some embodiments, the first dose is about 1 mg and the second dose is about 1 mg. In some embodiments, the first dose is about 10 mg and the second dose is about 10 mg. In some embodiments, the first dose is about 25 mg and the second dose is about 25 mg. In some embodiments, the first dose is about 10 mg and the second dose is about 25 mg. In some embodiments, the first dose is about 25 mg and the second dose is about 10 mg. In some embodiments, the first dose is about 1 mg and the second dose is about 10 mg. In some embodiments, the first dose is about 1 mg and the second dose is about 25 mg. In some embodiments, the first dose is about 10 mg and the second dose is about 1 mg. In some embodiments, the first dose is about 25 mg and the second dose is about 1 mg.

In some embodiments a second dose of psilocybin is administered from about one week to about 12 weeks after a first dose. In some embodiments, a second dose of psilocybin is administered about one week after a first dose. In some embodiments, a second dose of psilocybin is administered about two weeks after a first dose. In some embodiments, a second dose of psilocybin is administered about three weeks after a first dose. In some embodiments, a second dose of psilocybin is administered about four weeks after a first dose. In some embodiments, a second dose of psilocybin is administered about five weeks after a first dose. In some embodiments, a second dose of psilocybin is administered about six weeks after a first dose.

### Administration Routes

Exemplary modes for administration of psilocybin include oral, parenteral (e.g., intravenous, subcutaneous, intradermal, intramuscular [including administration to skeletal, diaphragm and/or cardiac muscle], intradermal, intrapleural, intracerebral, and intra-articular), topical (e.g., to both skin and mucosal surfaces, including airway surfaces, and transdermal administration), inhalation (e.g., via an aerosol), rectal (e.g., via a suppository), transmucosal, intranasal, buccal (e.g., sublingual), vaginal, intrathecal, intraocular, transdermal, in utero (or in ovo), intralymphatic, and direct tissue or organ injection (e.g., to liver, skeletal muscle, cardiac muscle, diaphragm muscle or brain). In some embodiments, psilocybin is administered orally to the subject.

### Methods of Treatment

It is to be understood by one of skill in the art that the methods of treatment comprising administering psilocybin, a prodrug of psilocybin, a metabolite of psilocybin, and/or a prodrug of a metabolite of psilocybin for the treatment of one or more indications as described herein also include: the use of psilocybin, a prodrug of psilocybin, a metabolite of psilocybin, and/or a prodrug of a metabolite of psilocybin in the manufacture of a medicament for the treatment of one or more indications as described herein; and the use of psilocybin, a prodrug of psilocybin, a metabolite of psilocybin, and/or a prodrug of a metabolite of psilocybin for the treatment of one or more indications as described herein.

In some embodiments, a method for treating a subject in need thereof comprises administering to the subject a therapeutically effective dose of psilocybin. In some embodiments, a method for treating a subject in need thereof comprises administering to the subject a therapeutically effective dose of psilocybin in a controlled environment, wherein the subject is provided with psychological support.

In some embodiments, a method for treating a subject in need thereof comprises at least one of the following:
(i) administering to the subject a therapeutically effective dose of psilocybin in a controlled environment, wherein the subject is provided with psychological support;
(ii) having the subject participate in one or more pre-administration psychological support session(s); and/or
(iii) having the subject participate in one or more post-administration psychological support session(s).

After administration of the psilocybin, the subject may not feel the effects of the drug for about 30 minutes to about 90 minutes. In some embodiments, the subject may not feel the effects of the drug for about 60 minutes. This period after administration and before the onset of effects will be referred to herein as the initial stage of the psilocybin session. The time marked by the onset of the drug's effects will be referred to herein as the early stage of the psilocybin session.

In some embodiments, the subject will experience the peak of the psilocybin's effects at about 1.5 hours to about 3.5 hours after administration thereof. The time period marked by the peak psilocybin experience will be referred to herein as the peak stage of the psilocybin session.

In some embodiments, the effects of the psilocybin may substantially wear off from about 4 hours to about 6 hours after administration. This time period will be referred to as the late stage of the psilocybin session.

In some embodiments, the subject's ability to reach a non-dual state (e.g., a mystical experience), or a sense of unity, boundlessness, ego-dissolution or transcendence correlates with positive clinical outcome. Each of these terms may be commonly defined as the breakdown of the usual relationship between self and other, whereby the subject might feel a oneness and increased sense of connectedness to the surrounding environment and/or the world at large.

In some embodiments, low levels of emotional arousal-which could indicate avoidance, lack of involvement or intellectualization-might, in some embodiments, be correlated with little or no improvement in treatment outcomes.

Factors that may influence the subjective experience of psilocybin include, for example, (i) dose, (ii) the mindset of the participant prior to the session, (iii) the setting of the session, (iv) the subject's ability to focus and stay with the experience, and/or (v) the subject's prior experience with psychedelics. These, and other factors, will be described in more detail below, along with ways to maximize therapeutic benefit of the psilocybin session.

### Pre-Administration Psychological Support Sessions

In some embodiments, the subject participates in at least one psychological support session before administration of the psilocybin (“pre-administration psychological support session”). In some embodiments, a pre-administration psychological support session may be held about 1 month prior to the psilocybin administration. In some embodiments, a pre-administration psychological support session may be held about 2 weeks prior to the psilocybin administration. In some embodiments, a pre-administration psychological support session may be held about 1 week prior to the psilocybin administration. In some embodiments, a pre-administration psychological support session may be held about 3 days prior to the psilocybin administration. In some embodiments, a pre-administration psychological support session may be held about 1 day prior to the psilocybin administration. In some embodiments, a pre-administration psychological support session may be held on the same day as and prior to psilocybin administration.

In some embodiments, the subject may participate in one, two, three, four, five, six, seven, or eight pre-administration psychological support sessions. In some embodiments, the subject may participate in at least two pre-administration psychological support sessions. In some embodiments, the subject may participate in at least three pre-administration psychological support sessions. In some embodiments, the subject may participate in pre-administration psychological support sessions at least once per week, for at least two or three weeks prior to the psilocybin session. In some embodiments, the subject may additionally participate in a pre-administration psychological support session the day before the psilocybin session.

The pre-administration psychological support sessions may be individual sessions, wherein a subject meets one-on-one with a therapist. In some embodiments, the psychological support sessions may be group sessions, wherein more than one subject meets with a single therapist, or more than one therapist. In some embodiments, one or more of the subject's family members or friends may be present at the pre-administration psychological support session(s).

In some embodiments, the goals of the pre-administration session may include (i) establishing therapeutic alliance between subject and therapist; (ii) answering the subject's questions and addressing any concerns; and/or (iii) demonstrating and practicing the skills of self-directed inquiry and experiential processing. In some embodiments, the pre-administration psychological support sessions focus on discussion of possible psilocybin effects, and/or preparing subjects for the dosing session by practicing relevant therapeutic techniques to reduce avoidance and anxiety, eliciting relevant therapeutic goals, building rapport, and/or establishing therapeutic alliance. During the psychological support session, skills of self-directed inquiry and experiential processing may be demonstrated and/or practiced.

In some embodiments, breathing exercises meant to promote calm and/or ease anxiety may be demonstrated and/or practiced. In some embodiments, the breathing exercise comprise instructing the subject to focus on their breath and/or sensations associated with the breath throughout the body. For example the subject may be instructed to breathe in for a count of four, to hold their breath for a moment, and then to breathe out for a count of eight. In some embodiments, the therapist and subject may discuss the most helpful ways to support in case of emotional distress during the psilocybin session. In some embodiments, the subject is given access (e.g., online access) to materials concerning the safety and mechanism of action of psilocybin.

In some embodiments, the pre-administration psychological support sessions will serve to establish a therapeutic goal for the psilocybin session. In some embodiments, the subject suggests the therapeutic goal for herself or himself. In some embodiments, the therapist suggests the therapeutic goal to the subject. In some embodiments, the subject is reminded of the therapeutic goal during the pre-administration psychological support session.

In some embodiments, the therapists are trained to counsel the subject before, during, and/or after the psilocybin sessions. In some embodiments, the therapist will have mental health training. In some embodiments, the therapist will be a clinical psychologist, a psychiatrist, a social worker, a doctor or a nurse. In some embodiments, the therapist will meet the following criteria:
Demonstrate independent clinical experience with direct subject care in areas that require counselling and psychotherapeutic skills;
Current unrestricted professional license and/or good professional standing with no history of suspension, professional misconduct or disciplinary actions; and/or
High level of openness to learning new approaches and receiving feedback.

### Psychological Support During Psilocybin Sessions

During the treatment session, the subject may be supervised by one or more trained therapists. The therapist supervising the subject during the psilocybin session may be the same therapist from the subject's pre-administration psychological support session(s), or may be a different therapist. The therapist(s) may provide psychological support to the subject as necessary. As used herein, the term “psychological support” refers to any measure(s) taken by the therapist during the subject's psilocybin session to ensure the safety of the subject and maximize the clinical effectiveness of the psilocybin session. For example, the psychological support may be anything done by the therapist to (1) to ensure psychological safety of the subject; (2) to allow the subject's subjective experience to unfold naturally within the boundaries of the therapeutic intention set at the preparation; (3) to maintain participant's attention and awareness on the experience of the present moment thus allowing exposure and processing of the challenging emotional states and personal memories; and/or (4) to generate insights and solutions for the resolution of challenging personal situations, conflicts and traumatic experiences. In some embodiments, support can be in the form of therapeutic touch, verbal reassurance, guided imagery and/or relaxation or breathing exercises. In some embodiments, the support may comprise reminders, encouragement, or active guiding. Typically, only one technique is applied at a time to allow for minimal intervention and interference with the subject's unique process.

In some embodiments, the main therapeutic goals of the therapist during the psilocybin session are to (i) minimize extreme anxiety, and (ii) provide appropriate support that enables the skills and processes of self-directed inquiry and experiential processing. In some embodiments, the therapist demonstrates genuine presence, patience, curiosity, and/or openness during the psilocybin session. “Presence” refers to being totally available and present with the subject during all stages of the psilocybin session, and exuding calmness at all times. “Curiosity” refers to interest and willingness to understand the subject's experience, without making assumptions. “Patience” means that the therapist facilitates the participant taking as much time as needed to explore their experiences without controlling the natural urge to help or direct the experience. “Openness” is the ability of the therapist to remain cognitively and experientially open, including a capacity to be curious about how the subject's mind may uniquely choreograph the unfolding content of a session. This includes welcoming all emotions and expressions that might occur.

In some embodiments, the psychological support may comprise curious questioning. In this technique, brief, but detailed, questioning of subjects is used to help the subjects shift and sustain their attention towards different levels of cognition and emotions (“How does that make you feel?”) Due to the applicability across a range of mental states and within various settings, the technique of curious questioning can typically be used safely and consistently during the psilocybin session, regardless of the quality or intensity of the experience of each subject.

In some embodiments, the level of psychological support will vary during the various stages of the subject's psilocybin experience (e.g., the initial stage, the early stage, the peak stage, and the late stage). In some embodiments, the type of psychological support will vary during the various stages of the subject's psilocybin experience (e.g., the initial stage, the early stage, the peak stage, and the late stage). Because non-dual, ego-dissolution or “unitive” experiences have been shown to positively correlate with the magnitude and durability of the clinical response, the therapist will, in some embodiments, attend to such states with particular care.

In some embodiments, a subject may experience of a compromised sense of self during the subject's psilocybin experience. In some embodiments, this is interpreted from a psychoanalytic perspective as a disruption of ego-boundaries, which results in a blurring of the distinction between self-representation and object-representation, and precludes the synthesis of self-representations into a coherent whole. In some embodiments, non-dual, ego-dissolution or “unitive” experiences refer to an altered state of consciousness in which there is a reduction in the self-referential awareness that defines normal waking consciousness, resulting in a compromised sense of “self” and instead only a undivided background awareness, often characterised by a sense of unity or “oneness” that exceeds sensory or cognitive apprehension. In some embodiments, a non-dual experience is state of consciousness in which the subject-object dichotomy in normal waking consciousness is substituted for a unified background awareness that is centreless and undivided. In some embodiments, an ego dissolution experience is a spontaneously occurring state of consciousness where there is a reduction in the self-referential awareness that defines normal waking consciousness, resulting in a compromised sense of “self”. In some embodiments, a unitive experience is an experience characterised by a sense of unity or “oneness” that exceeds sensory or cognitive apprehension.

At the initial and early stage of the psilocybin session, psychological support may be used to reduce severe and/or prolonged anxiety. Anxiety prior to or during the onset of psilocybin effects is not uncommon, and the therapists may be specially trained to recognize and actively manage subjects through such periods of anxiety until the subject is comfortable enough to continue on their own. In some embodiments, therapists validate the subject's feelings of anxiety without providing interpretations of perceptual disturbances or guiding subjects towards a particular image or memory, other than encouraging them to stay relaxed and open to the emergent experiences. For example, in some embodiments, the therapist may help alleviate anxiety using a grounding exercise. In such an exercise, the subject may be encouraged to pay attention to the sounds around them or to sensations on their skin when touching the bed/couch, ground, or other objects.

At the initial and early stage of the psilocybin session, the therapist may encourage the subject to lie down, practice relaxation and breathing exercises, and/or listen to calming music. In some embodiments, the therapist may remind the subject of the intention for the treatment session. For example, the therapist may ask the subject “What does feeling better or recovery feel like?” or any number of similar questions. Such reminders prior to the onset of or at the onset of psilocybin effects provide an implicit direction for the subjective experience during the psilocybin session. In some embodiments, the therapist may remind the subject that their primary task during this session is to simply collect new and interesting experiences which can then be discussed with the therapist after the session. The therapist may remind the participant of the purpose of the psilocybin therapy and the role of experiential processing, namely allowing the participant to be open and curious to whatever arises and encountering thoughts and feelings previously unknown to them. In some embodiments, the therapist emphasizes that this process inherently requires letting go and a willing passivity to the psychedelic experience.

During the acute onset of action, the subject might experience perceptual changes in visual, auditory or olfactory modes, and a range of unusual physical sensations. These experiences could be anxiety-provoking. In some embodiments, the therapist may practice reassuring “arm holding”. This is where, upon the subject's request, a therapist will place his or her hand on the subject's wrist, arm, hand, or shoulder, as a way of helping the subject feel secure during this phase. This exercise may have been previously practiced during the pre-administration psychological support session.

In some embodiments, the therapist may encourage the subject to put on an eye mask, such as a Mindfold eyeshade. In some embodiments, the therapist encourages the subject to put on the eye mask before, during, or after the onset of the psilocybin's effects.

In some embodiments, the therapist may encourage the subject to put on headphones and listen to music. In some embodiments, the headphones reduce outside noise (e.g., “noise-cancelling” headphones). In some embodiments, the music is calming music such as instrumental (e.g., classical) music. In some embodiments, the music comprises nature sounds and/or the sound of moving water (e.g., ocean sounds). In some embodiments, the music comprises isochronic tones. In some embodiments, the music comprises moments of silence. In some embodiments, the music is emotionally evocative. In some embodiments, the music comprises a playlist which mirrors the pharmacodynamics of a typical high-dose psilocybin session: the initial stage, the early stage, the peak stage, and the late stage. In some embodiments, listening to music helps the subject to focus on their internal experience.

In case of prolonged anxiety or distress, therapists may, in some embodiments, actively guide participants through such experiences without interpreting or judging the experiences or giving advice. Once participants are comfortable, the therapist may encourage them to again engage in introspection.

During the peak and late stages of the psilocybin session, the therapist may encourage subjects to face and explore their experience, including the challenging ones. Therapists may direct subjects to participate self-directed inquiry and experiential processing to develop a different perspective on their personal challenges and conflicts, and to generate their own solutions. Such self-generated insights are not only therapeutic because of the emotional resolution, but also empowering to subjects.

As used herein, the term “self-directed inquiry” refers to directing attention to internal states. Subjects are encouraged to be curious about experiences in the present moment, including foreground and background thoughts, emotions, and physical sensations. During the preparation and integration stages, this inquiry might mean asking specific and detailed questions to help direct attention to internal states. However, during the period of drug action, inquiry might simply mean an attitude of openness to inner experiences.

As used herein, “experiential processing” refers to a participant's ability to maintain full attention on the experiences that come into awareness through self-directed enquiry. This includes a willingness and ability to be with and/or move ‘in and through’ even uncomfortable or challenging thoughts, feelings, sensations or emotions, until discomfort is diminished or resolved.

In some embodiments, the therapist will employ a transdiagnostic therapy. In some embodiments, the transdiagnostic therapy is a Method of Levels (MOL) therapy. In still further embodiments, the MOL therapy comprises Self-Directed Enquiry and Experiential Processing. Typically, MOL uses brief, but detailed, curious questioning to help subjects shift and sustain their attention towards different levels of cognition and emotions (Carey, 2006; Carey, Mansell & Tai, 2015). The emphasis within MOL is on identifying and working with a subject's underlying distress as opposed to just their symptoms. Such MOL related methods and techniques can include: (1) Self-directed enquiry—directing attention to internal states. Participants are encouraged to be curious about experiences in the present moment, including foreground and background thoughts, emotions, and physical sensations; during the preparation and integration stages, such enquiry can mean asking specific and detailed questions to help direct attention to internal states, although for some embodiments, during the period of drug action, enquiry can refer to an attitude of openness to inner experiences; and (2) Experiential processing-sustained focus on the experience; refers to a participant's ability to maintain full attention on the experiences that come into awareness through self-directed enquiry. This includes a willingness and ability to be with and/or move ‘in and through’ even uncomfortable or challenging thoughts, feelings, sensations or emotions, until discomfort is diminished or resolved.

In some embodiments, the psychological support comprises mindfulness-based therapy or CBT cognitive behavioral therapy (CBT). In some embodiments, the psychological support is informed by a functional theory of human behavior called Perceptual Control Theory.

Occasionally, the subject will try to avoid emerging experiences or distract him/herself while trying to regain cognitive control over the unusual state of their mind. Such distractions may take different forms. For example, the subject might want to engage in a conversation or prematurely describe in detail their experience, visions or insights. When this occurs, the therapist may aim to remain as silent as possible, thereby enabling the subject and his/her inner experience to direct the course of the psilocybin session. In some embodiments, the therapist may use active listening skills paired with prompts to encourage the subject to continue focusing attention on present experiences, particularly if the participant engages the therapist in conversation. In another example, a subject might ask to go to the bathroom or have a drink of water. The sudden and urgent character of such requests might suggest that they are really trying to avoid emerging material. In such cases, the therapist may encourage the subject to stay with the experience by simply redirecting their attention. For example, the therapist may say something like, “We will take a bathroom break at the end of this piece of music” or “I will get you water in a little while. Why don't you put the eye shades back on and relax for a few minutes?” If the subject is trying to avoid a difficult experience, they might listen to the suggestion and relax.

In some embodiments, spontaneous movement such as shaking, stretching or dancing while engaging with the experience is accepted and often encouraged, unless the movement seems to be a way to distract oneself from the experience. In some embodiments, if the subject continues to move around a lot, reminders to periodically return to a lying down position and to actively focus inwards may be provided.

The therapist is not required to understand, support or even have an opinion about the nature or content of the subject's experiences, but the therapist may validate them and convey openness toward the subject's own view of them without dismissing or pathologizing any experience based on its unusual content. These experiences may provide the subject with a perspective that goes beyond identification with their personal narrative. In some embodiments, the therapist will validate one or more of the subject's experiences. In some embodiments, validation of the experiences simply means acknowledging the courage of opening up to the experience and the possibility that any experience will serve the intention of the session.

In some embodiments, a therapist provides psychological support for approximately 4-8 hours immediately after administration of the psilocybin. In some embodiments, the therapist uses guided imagery and/or breathing exercises to calm the subject and/or focus the subject's attention. In some embodiments, the therapist holds the hand, arm, or shoulder of the subject. In some embodiments, the therapist counsels the subject to do one or more of the following: (1) to accept feelings of anxiety, (2) to allow the experience to unfold naturally, (3) to avoid psychologically resisting the experience, (4) to relax, and/or (5) to explore the subject's own mental space.

In some embodiments, the therapist avoids initiating conversation with the subject, but responds if the subject initiates conversation. Typically, active intervention is kept to a minimum during the treatment experience. In some embodiments, the subject is encouraged to explore their own mental space, and simple guided imagery may be used to assist relaxation. “Guided imagery” refers to an exercise wherein the subject is asked to imagine a scene (e.g., “Invite a scene, perhaps a landscape, and tell me where you find yourself”; “Imagine a place that feels safe to you.”)

### Post-Administration Psychological Support Session

In some embodiments, subjects may be encouraged to engage in post-administration integration sessions with their therapist. Integration is a process that involves processing, or embodying, a psychedelic experience within a therapeutic context. The process initially begins by the subject verbalising and reflecting upon any experience from the psilocybin session, and discussing it openly with their therapist. Successful integration of a psilocybin experience accommodates for emotional changes and comprises of translating experiences into new insights, perspectives, and subsequently new behaviors that can be used to benefit the subject's quality of life. New perspectives might in turn influence the participant's current knowledge or values and lead to new ways of relating to cognitions, emotions, behaviors and physical experiences.

In some embodiments, the goals and supportive methods used by the therapist throughout integration sessions should remain consistent, regardless of the intensity or content of the subjective experience explored by the subject. That said, the methods of support used by the therapist should accommodate for the full range of experiences a subject might have faced.

The integration process is not one that should be limited to the sessions with the therapist, and is a process that will likely continue to unfold beyond the visits in clinic. The therapist might encourage the participant to use methods such as spending time in nature, exercise, or creative expression to help facilitate the process further. The subject might also be encouraged to discuss experiences with their friends, family, and/or support network. The role of the integration sessions is not to cover and work on every experience, but to empower the participant by building their capacity to experientially process information safely. This enables the patient to continue self-directed integration, even outside of study visits.

In some embodiments, the subject participates in at least one psychological support session after administration of the psilocybin (“post-administration psychological support session”). In some embodiments, a post-administration psychological support session may be held on the same day as the psilocybin session, after the effects of the psilocybin have substantially worn off. In some embodiments, a post-administration psychological support session may be held the day after the psilocybin session. In some embodiments, a post-administration psychological support session may be held two days after the psilocybin session. In some embodiments, a post-administration psychological support session may be held three days after the psilocybin session. In some embodiments, a post-administration psychological support session may be held about one week after the psilocybin session. In some embodiments, a post-administration psychological support session may be held about two weeks after the psilocybin session. In some embodiments, a post-administration psychological support session may be held about one month after the psilocybin session. In some embodiments, a post-administration psychological support session may be held about three months after the psilocybin session. In some embodiments, a post-administration psychological support session may be held about six months after the psilocybin session. In some embodiments, a post-administration psychological support session may be held about twelve months after the psilocybin session.

In some embodiments, the subject may participate in one, two, three, four, five, six, seven, or eight post-administration psychological support sessions. In some embodiments, the subject may participate in at least two, or at least three post-administration psychological support sessions.

The post-administration psychological support sessions may be individual sessions, wherein a subject meets one-on-one with a therapist. In some embodiments, the psychological support sessions may be group sessions, wherein more than one subject meets with a single therapist, or more than one therapist. In some embodiments, one or more of the subject's family members or friends may be present at the post-administration psychological support session(s).

In some embodiments, the post-administration psychological support session may focus on integration of the psilocybin experience. Integration may involve processing a psychedelic experience in a therapeutic context. Integration may comprise psychological and somatic processing of the experience and a successful assimilation of insights into the subject's life for the purpose of growth, healing and/or well-being. During an integration session, a subject may be encouraged to talk about and reflect upon their experiences during the psilocybin session. In some embodiments, integration may comprise an external expression of the psilocybin experience, such as choice of words, tone of voice, gestures, and/or particular physical activities (yoga, exercise, bodywork, etc.) In some embodiments, integration comprises creatively expressing any insights or experiences gained during a psilocybin experience, for example through poetry, art, music/singing, dance, writing or drawing.

In some embodiments, the subject may be encouraged to reflect on both the thoughts and the feelings that he or she underwent during the psilocybin session, as well as to express those ideas and emotions into a concrete form that can serve as a tool for continuing to remember and integrate those lessons into the future. In some embodiments, the subject may be encouraged to acknowledge and connect with the range of the emotional cognitive and physical experiences of the psilocybin session, and relate them to current experiences in their life situation. This may be accomplished, for example, by discussing them initially with their therapist, and perhaps later with their family, friends, and support circle. Integration helps accommodate changes in emotional states as new insights are generated and integrated. When further explored through oscillating attention between foreground and background thoughts and emotions, such insights may lead to natural and effortless changes in perspectives or behaviors. In some embodiments, the integration process is not limited to initial integration meetings with the therapist, but continues to unfold spontaneously through a participant's own processing and actions in everyday life.

In the case of a low-intensity experience, the integration process might focus on the mental content that emerged during the hours of relaxation and introspection. This might also include reactions to what might have been an unremarkable experience, such as feeling of disappointment, anger, relief etc.

### Psychological Support Provided Remotely

In some embodiments, psychological support may be provided remotely to a subject. For example, a therapist providing psychological support may not be in the same room, the same building, or in the same facility as a subject. Remote psychological support may be provided, for example by telephone (i.e., by voice call), by video call or video conference, by text, or by email.

In some embodiments, a pre-administration therapy session is conducted remotely. In some embodiments, a post-administration therapy session (e.g., an integration session) is conducted remotely.

In some embodiments, psychological support is provided remotely during the subject's psilocybin session. For example, in some embodiments, the subject takes the psilocybin in his or her own home, and a therapist provides psychological support by voice call, video call, text, email, etc., for at least 4-8 hours after the subject has taken the drug. In some embodiments, the subject takes the psilocybin in an administration facility as described herein, and the therapist provides psychological support to the subject a therapist provides psychological support by voice call, video call, text, email, etc., for at least 4-8 hours after the subject has taken the drug

In some embodiments, remote psychological support is provided to the subject using a digital or electronic system. In some embodiments, the digital or electronic system may comprise one or more of the following features:
The digital or electronic system securely connects patients with one or more therapists or physicians for “virtual visits.” These virtual visits may be introductory or routine.
The digital or electronic system allows a subject to qualify, prequalify, or register for a psilocybin-based clinical trial, or a psilocybin-based psychological support session.
The digital or electronic system is configured to help therapists and/or physicians manage and interact with patients. For example, the electronic system may allow the therapist to share documents with subjects, keep notes about sessions, or schedule future sessions.
The digital or electronic system is configured to provide alerts for crisis intervention. For example, the digital or electronic system may allow the subject to contact the therapist if they are feeling anxiety or otherwise urgently need to talk to the therapist.
The digital or electronic system is configured to help prepare the subject for a visit with their therapist and/or physician. For example, the digital or electronic system may contain information regarding psilocybin, the therapeutic protocol, etc.
The digital or electronic system is configured to allow the therapist to provide psychological support during the subject's psilocybin session. For example, the system may comprise a video calling or chat feature.
The digital or electronic system is configured to allow the therapist to provide psychological support during a post-administration session (e.g., an integration session).
The digital or electronic system is configured to track the subject's adherence to the treatment regimen or goals.
The digital or electronic system is configured to assess one or more clinical endpoints in the subject. For example the system may comprise one or more questionnaires or exercises for the subject to complete. Results may be made available to the subject's physician and/or therapist.

In some embodiments, the digital or electronic system is an “app” for use on a mobile phone or a computer. In some embodiments, the digital or electronic system is a website. In some embodiments, the digital or electronic system comprises a “chat” feature which allows communication between the subject and the therapist in real time. In some embodiments, the website comprises a video calling feature, which allows for the therapist to communicate with the subject using video communication. In some embodiments, the digital or electronic system is configured to allow a single therapist to provide psychological support to one or more subjects at or around the same time.

In some embodiments, psychological support sessions may be pre-recorded (e.g., audio or video recording) and provided to the subject for use at the subject's convenience via the digital or electronic system.

### Administration Facility, “Set and Setting”

As used herein, the term “set and setting” refers to the subject's mindset (“set”) and the physical and social environment (“setting”) in which the user has the psilocybin session. In some embodiments, the psilocybin may be administered in a particular set and setting. In some embodiments, the set and setting is controlled, to the extent possible, to maximize therapeutic benefit of the psilocybin session.

In some embodiments, the psilocybin is administered by in a facility specifically designed for psilocybin administration. Administration of the psilocybin to the subject in a facility where the subject feels safe and comfortable may help ease anxiety in the subject, and may facilitate maximum clinical benefit. Psilocybin may be administered to a subject, for example, in the subject's home or at a clinical facility.

In some embodiments, the psilocybin is administered to the subject in a facility (e.g., a room) with a substantially non-clinical appearance. For example, the psilocybin can be administered in a room that comprises soft furniture (e.g., plush couches, chairs, or pillows) and/or plants. In some embodiments, the room may be decorated using muted colors (e.g., greyed, dulled, or desaturated colors). In some embodiments, the light in the room is dimmed and/or light levels are kept or adjust to be relatively low. In some embodiments, the room lighting is adjusted for intensity and/or color. In some embodiments, a virtual reality or augmented reality system (e.g., computer with visual/graphical and auditory outputs) is used. In some embodiments, the room comprises a sound system, for example a high-resolution sound system. In some embodiments, the sound system can allow for simultaneous ambient and earphone listening. In some embodiments, the subject may bring meaningful photographs or objects into the administration room.

In some embodiments, the room comprises a couch. In some embodiments, the room comprises a bed. In some embodiments the room comprises more than one couch or bed, such as 2, 3, 4, 5, 6, 7, 8, 9, or 10 couches or beds. In some embodiments, the subject sits on or lies in the couch or bed for approximately 4-8 hours, or a substantial fraction thereof, immediately after administration of the psilocybin. In some embodiments, the subject listens to music for approximately 4-8 hours, or a substantial fraction thereof, immediately after administration of the psilocybin. In some embodiments, the subject wears an eye mask for approximately 4-8 hours, or a substantial fraction thereof, immediately after administration of the psilocybin. In some embodiments, the subject is provided with a weighted blanket.

In some embodiments, each subject is supervised by one therapist during the psilocybin session. In some embodiments, each subject is supervised by more than one therapist during the psilocybin session, such as two therapists, three therapists, four therapists, or five therapists. In some embodiments, one therapist multiple subjects, wherein each subject is participating in a psilocybin session. For example, one therapist may supervise two, three, four, five, six, seven, eight, nine, or ten subjects.

Embodiments of the disclosure include use of additional tools and/or technique(s) with dosage/administration, including various transcranial magnetic stimulation (TMS) methods and protocols, for example, prior or subsequent to one or more dosing(s), biofeedback devices, etc.

Some embodiments can be used with a digital health product or digital solution. Teachings of the disclosure include utilization of such digital health products and/or related digital biomarkers as diagnostic and/or prognostic tools for patient monitoring and management pre-treatment, during treatment, and/or post treatment. Digital biomarkers can include, by way of non-limiting example: Number of and/or time of phone calls/e-mails/texts; word length in text communication; Gestures used (taps, swipes, or other); Gyroscope derived information e.g. orientation of the phone; Acceleration of the phone; Keystroke patterns; Location derived information from GPS; facial expressions and/or microexpressions; voice or vocal markers; natural language processing; social media use; sleep patterns; specific words or emojis used or not used; and/or the like. For example, in one embodiment, a digital health product can be utilized to determine dosing amount and/or dosing frequency, indicator of a need for re-dosing, re-dosing amount, a warning or alert, as tracking of compliance, etc.

In some embodiments, methods of treatment can include providing a clearance time for a subject or patient, such one or more medications is not present or substantially cleared from the system of the subject/patient. For example, methods of treatment can be configured such that, upon administration, the subject is not taking other serotonergic medications such as: selective-serotonin reuptake inhibitors, selective norepinephrine reuptake inhibitors, tricyclic antidepressants, monoamine oxidase inhibitors and/or antipsychotics. In some embodiment, the method of treatment include treatment concurrently with one or more medications, including but not limited to selective-serotonin reuptake inhibitors, selective norepinephrine reuptake inhibitors, tricyclic antidepressants, and/or monoamine oxidase inhibitors. In some embodiments, the method include treatment such that subjects or patients take concomitant compounds or medications, including but not limited to benzodiazepines, cannabidiol (CBD) and/or other cannabinoids (e.g., THC (tetrahydrocannabinol); THCA (tetrahydrocannabinolic acid); CBD (cannabidiol); CBDA (cannabidiolic acid); CBN (cannabinol); CBG (cannabigerol); CBC (cannabichromene); CBL (cannabicyclol); CBV (cannabivarin); THCV (tetrahydrocannabivarin); CBDV (cannabidivarin); CBCV (cannabichromevarin); CBGV (cannabigerovarin); CBGM (cannabigerol monomethyl ether); CBE (cannabielsoin); CBT (cannabicitran); and/or the like) magnesium, Levomefolic acid, e.g., for a period of time prior to, just prior to, and/or at the same time as receiving psilocybin.

In some embodiments, the method includes treatment such that a subject has not taken one or more medications, particularly has not taken one or more serotonergic medications for at least 2 days, at least, 3 days, at least 4 days, at least 5 days, at least six days, at least 1 week, at least 2, 3, or 4 weeks before administration of the disclosed psilocybin compound.

In some embodiments, the method and/or treatment can comprise subperceptual-dosing (e.g., a dose of less than 3 mg, 2.5 mg, 2 mg, 1.5 mg, 1 mg, 0.9 mg, 0.8 mg, 0.7 mg, 0.6 mg, 0.5 mg, 0.4 mg, 0.3 mg, 0.2 mg, or 0.1 mg) prior to and/or following the administration of a relatively larger single dose or multiple doses (given a few days to a few weeks apart), where the relatively larger single dose or multiple doses is one or more of 5 mg or more, 10 mg or more, 15 mg or more, 20 mg or more, 25 mg or more, 30 mg or more, 35 mg or more, 40 mg or more, 45 mg or more, 50 mg or more.

Embodiments of the disclosure include method utilizing a digital biomarker, for example, as a diagnostic and/or prognostic tool for patient management pre-, during and/or post treatment with psilocybin wherein the digital biomarker is one or more biomarkers associated with executive function, cognitive control, working memory, processing speed, and/or emotional valence.

In some embodiments the digital biomarker is identified from patterns in smartphone use such as swipes, taps, and other touchscreen activities, and can be scientifically validated to provide measurements of subject status, such as cognition and mood, including, by way of non-limiting example, as disclosed in one or more of the following, each of which is herein expressly incorporated by reference for all purposes: US20170086727, US20170258382, US20170258383, US20170287348, U.S. Pat. Nos. 10,148,534, 9,737,759, and/or 10,231,651.

Biomarkers which may serve as a diagnostic and/or prognostic tool for patient management pre, during and/or post treatment may be identified using one or more of: Number of and/or time of phone calls/e-mails/texts; word length in text communication; Gestures used (taps, swipes, or other); Gyroscope derived information e.g. orientation of the phone; Acceleration of the phone; Keystroke patterns; Location derived information from GPS; facial expressions and/or microexpressions; voice or vocal markers; natural language processing; social media use; sleep patterns; specific words or emojis used or not used; and/or the like. In some embodiments, health components and/or connected biomonitors and/or smart devices/wearables can be utilized to collect information to be used in diagnostic and/or prognostic outputs. For example, in some embodiments, a heart rate monitor or similar device can collect a subject's data and heart rate variability (for example only, as disclosed in U.S. Pat. No. 10,058,253, the entirety of which is herein incorporated by reference) can be used to assess/determine a metric relating to the subject's current emotional state, relative change in emotional state, etc., which can be used in determining a new or follow-on treatment plan, adjusting a treatment plan, etc.

In accordance with a further aspect of the disclosure there is provided a method of assessing a subject pre, during and/or post treatment of a central nervous system disorder to determine whether to provide a psilocybin treatment or a further psilocybin treatment comprising monitoring one or more biomarkers associated with executive function, cognitive control, working memory, processing speed, and emotional valence, and determining the treatment based on an outcome. The method can further comprise the step of administering psilocybin for a first or a subsequent time.

In some embodiments, the biomarker is identified from patterns in smartphone use such as swipes, taps, and other touchscreen activities, and are scientifically validated to provide measurements of cognition and mood. For example, in some instances, the pattern is identified using one or more of: Number of and/or time of phone calls/e-mails/texts; word length in text communication; Gestures used (taps, swipes, or other); Gyroscope derived information e.g. orientation of the phone; Acceleration of the phone; Keystroke patterns; Location derived information from GPS; facial expressions and/or microexpressions; voice or vocal markers; natural language processing; social media use; sleep patterns; specific words or emojis used or not used; and/or the like.

Embodiments include a method of assessing a subject pre, during and/or post treatment of a central nervous system disorder to determine whether to provide a psilocybin treatment or a further psilocybin treatment comprising monitoring one or more biomarkers associated with executive function, cognitive control, working memory, processing speed, and emotional valence, and determining the treatment based on an outcome; the method can further comprise administering psilocybin for a first or a subsequent time.

In some embodiments, the disclosure provides for treating 2 or more subjects, the method comprising administering to each subject a therapeutically-effective dose of psilocybin at the same time or substantially the same time (e.g., dosed within several minutes of each other, within 5, 10, 15, 20, 25, or 30 min of each other), wherein each subject is aware of the other subject also receiving treatment. In some embodiments, the subjects are in the same room. In some embodiments, the subjects are in different rooms.

In some embodiments, the disclosure provides a method of treating a subject, the method comprising administering to the subject a therapeutically-effective dose of psilocybin, and providing a virtual reality/immersive reality digital tool. In some embodiments, the light in the room is dimmed and/or light levels are kept or adjusted to be relatively low. In some embodiments, darkened glasses or eye shades are provided. In some embodiments, the room lighting is adjusted for intensity and/or color. In some embodiments, a virtual reality or augmented reality system (e.g., computer with visual/graphical and auditory outputs) is used.

### Subjects

In some embodiments, the subject is a male. In some embodiments, the subject is a female. In some embodiments, the female subject is pregnant or post-partum. In some embodiments, the subject is attempting to reduce or eliminate their use of a pharmaceutical agent, such as an anti-depressant or an anti-epileptic drug. In some embodiments, the subject is attempting to reduce or eliminate their use of the pharmaceutical agent before becoming pregnant, having surgery or other medical procedure, or starting to use different pharmaceutical agent.

The subject may be a geriatric subject, a pediatric subject, a teenage subject, a young adult subject, or a middle aged subject. In some embodiments, the subject is less than about 18 years of age. In some embodiments, the subject is at least about 18 years of age. In some embodiments, the subject is about 5-10, about 10-15, about 15-20, about 20-25, about 25-30, about 30-35, about 35-40, about 40-45, about 45-50, about 50-55, about 55-60, about 60-65, about 65-70, about 70-75, about 75-80, about 85-90, about 90-95, or about 95-100 years of age.

The subject may have a chronic disease or a terminal disease. In some embodiments, the subject may have a life-altering disease or condition (such as the loss of a limb or onset of blindness).

The subject may have recently been diagnosed with a disease, disorder, or condition. For example, the subject may have been diagnosed within 1 month, within 3 months, within 6 months, or within 1 year. In some embodiments, the subject may have been living with a disease, disorder, or condition for an extended period time, such as at least 6 months, at least 1 year, at least 3 years, at least 5 years, or at least 10 years.

In some embodiments, the subject may be a cancer patient, such as a Stage 4 or terminal cancer patient. In some embodiments, the subject may have been determined to have a limited time to live, such as less than 1 year, less than 6 months, or less than 3 months.

The subject may have previously taken a psychedelic drug, or may have never previously taken a psychedelic drug. For example, the subject may or may not have previously taken psilocybin, a psilocybin mushroom (“magic mushroom”), LSD (lysergic acid diethylamide or acid), mescaline, or DMT (N,N-Dimethyltryptamine).

In some embodiments, the subject may have previously taken one or more serotonergic antidepressants (e.g., selective serotonin reuptake inhibitors (SSRIs)). In some embodiments, the subject has never previously taken a serotonergic antidepressant. In some embodiments, the subject has not taken any serotonergic antidepressants for at least 2 weeks, at least 4 weeks, or at least 6 weeks prior to receiving psilocybin.

In some embodiments, the subject may have previously received electroconvulsive therapy (ECT). In some embodiments, the subject has not received any ECT for at least 2 weeks, at least 4 weeks, or at least 6 weeks prior to receiving psilocybin.

The subject may have a medical condition that prevents the subject from receiving a particular medical therapy (such as an SSRI or ECT). In some embodiments, the subject may have previously had an adverse reaction to a particular medical therapy (such as an SSRI or ECT). In some embodiments, a prior medical therapy (such as an SSRI or ECT) was not effective in treating a disease, disorder, or condition in the subject.

Diseases, Disorders, and/or Conditions to be Treated

Provided herein are methods of treating a subject in need thereof, the method comprising administering to the subject a therapeutically-effective dose of psilocybin.

The methods described herein may be used to treat a variety of diseases, disorders, or conditions including particular psychiatric and neurological aspects of the diseases, disorders, or conditions.

In some embodiments, the disclosure provides a method of treating a subject in need thereof, the method comprising administering to the subject a therapeutically-effective dose of psilocybin or a metabolite thereof, wherein the subject has at least one or more of the following diseases, disorders, or conditions: Disruptive Mood Dysregulation Disorder, Major Depressive Disorder (MDD), Treatment Resistant Depression, Persistent Depressive Disorder (Dysthymia), Premenstrual Dysphoric Disorder, Substance/Medication-Induced Depressive Disorder, Post-Partum Depression, Depressive Disorder due to Another Medical Condition, Separation Anxiety Disorder, Selective Mutism, Specific Phobia, Social Anxiety Disorder (Social Phobia), Panic Disorder, Panic Attack, Agoraphobia, Generalized Anxiety Disorder, Substance-Medication-Induced Anxiety Disorder, Anxiety Disorder Due to Another Medical Condition, Somatic Symptom Disorder, Illness Anxiety Disorder (hypochondriac), Conversion Disorder (Functional Neurological Symptom Disorder), Factitious Disorder, Post-Traumatic Stress Disorder (PTSD), Adjustment Disorders, Acute Distress Disorder, Obsessive-Compulsive Disorder, Body Dysmorphic Disorder, Hoarding Disorder, Trichotillomania (Hair-Pulling) Disorder, Excoriation (Skin-Picking) Disorder, Substance/Medication-Induced Obsessive-Compulsive and Related Disorder, Obsessive-Compulsive and Related Disorder due to Another Medical Condition, Substance-Related Disorders, Alcohol-Related Disorders, Cannabis-Related Disorders, Hallucinogen-Related Disorders, Inhalant-Related Disorders, Cocaine-Related Disorders, Opioid-Related Disorders, Sedative-, Hypnotic-, or Anxiolytic-Related Disorders, Stimulant-Related Disorders, Tobacco-Related Disorders, Non-Substance-Related Disorders (Gambling or Gaming Disorder), Migraines, Cluster Headaches such as Chronic Cluster Headaches, Cyclical Vomiting, Tension-Type Headache, Dysphasia, Pica, Anorexia Nervosa, Bulimia Nervosa, Binge-Eating Disorder, Oppositional Defiant Disorder, Intermittent Explosive Disorder, Conduct Disorder, Antisocial Personality Disorder, Psychopathy, Pyromania, or Kleptomania.

In some embodiments, the disclosure provides a method of treating a subject in need thereof, the method comprising administering to the subject a therapeutically-effective dose of psilocybin, wherein the subject has at least one of the following diseases, disorders, or conditions: a Neurocognitive Disorder due to Alzheimer's, Lewy Bodies, Traumatic Brain Injury, Prion Disease, Human Immunodeficiency Virus (HIV) Infection, Parkinson's, Huntington's; concussion; Chronic Traumatic Encephalopathy (CTE); Language Disorder, Speech Sound Disorder (Phonological Disorder); Childhood-Onset Fluency Disorder (Stuttering); Social (Pragmatic) Communication Disorder; Tourette's Disorder; Persistent (Chronic) Motor or Vocal Tic Disorder; Amnestic Disorder Due to Known Physiological Condition (possibly in ECT shock-resistant subjects); Transient Cerebral Ischemic Attack, Cerebral Infarction, Cerebral Bleeding, Progressive Supranuclear Ophthalmoplegia, or Retrograde Amnesia.

In some embodiments, the disclosure provides a method of treating a subject in need thereof, the method comprising administering to the subject a therapeutically-effective dose of psilocybin, wherein the subject has at least one of the following diseases, disorders, or conditions: Autism, Autism Spectrum-Disorder, or Antisocial Personality Disorder.

In some embodiments, the disclosure provides a method of treating a subject in need thereof, the method comprising administering to the subject a therapeutically-effective dose of psilocybin, wherein the subject has at least one of the following diseases, disorders, or conditions: Attention-Deficit/Hyperactivity Disorder, Other Specified Attention-Deficit/Hyperactivity Disorder or Unspecified Attention-Deficit/Hyperactivity Disorder.

In some embodiments, the disclosure provides a method of treating a subject in need thereof, the method comprising administering to the subject a therapeutically-effective dose of psilocybin, wherein the subject has at least one of the following diseases, disorders, or conditions: Schizotypal (Personality) Disorder, Delusional Disorder, Schizophrenia, or Schizoaffective Disorder.

In some embodiments, the disclosure provides a method of treating a subject in need thereof, the method comprising administering to the subject a therapeutically-effective dose of psilocybin, wherein the subject has at least one of the following diseases, disorders, or conditions: Insomnia Disorder, Hypersomnolence Disorder, Narcolepsy, or Primary Central Sleep Apnea.

In some embodiments, the disclosure provides a method of treating a subject in need thereof, the method comprising administering to the subject a therapeutically-effective dose of psilocybin, wherein the subject has at least one of the following diseases, disorders, or conditions: Schizoid Personality Disorder, Schizotypal Personality Disorder, Antisocial Personality Disorder, Borderline Personality Disorder, or Obsessive-Compulsive Personality Disorder.

In some embodiments, the disclosure provides a method of treating a subject in need thereof, the method comprising administering to the subject a therapeutically-effective dose of psilocybin, wherein the subject has at least one of the following diseases, disorders, or conditions: Female Sexual Interest/Arousal Disorder, Male Hypoactive Sexual Desire Disorder, and Excessive Sexual Drive.

In some embodiments, the disclosure provides a method of treating a subject in need thereof, the method comprising administering to the subject a therapeutically-effective dose of psilocybin, wherein the subject has at least one of the following diseases, disorders, or conditions: Bipolar I Disorder, Bipolar II Disorder, or Cyclothymic Disorder.

In some embodiments, the disclosure provides a method of treating a subject in need thereof, the method comprising administering to the subject a therapeutically-effective dose of psilocybin, wherein the subject has at least one of the following diseases, disorders, or conditions: Age-Related Hearing Loss or Tinnitus.

In some embodiments, the disclosure provides a method of treating a subject in need thereof, the method comprising administering to the subject a therapeutically-effective dose of psilocybin, wherein the has at least one of the following diseases, disorders, or conditions: Multiple Sclerosis, Cranial Nerve Disorder, Neuromyelitis Optica, Bell's Palsy, Guillain Barre Syndrome, Demyelinating Disease of Central Nervous System, or Chronic Inflammatory Demyelinating Polyneuritis.

In some embodiments, the disclosure provides a method of treating a subject in need thereof, the method comprising administering to the subject a therapeutically-effective dose of psilocybin, wherein the subject suffers from pain, such as phantom pain, chronic pain, or pain associated with another disease or disorder.

In some embodiments, the disclosure provides a method of treating a subject in need thereof, the method comprising administering to the subject a therapeutically-effective dose of psilocybin, wherein the subject has at least one of the following diseases, disorders, or conditions: Myelopathy, Traumatic Brain Injury, Intellectual Disabilities, Mania, Neurodegeneration, Paraphilic disorders (e.g., Pedophilic Disorder), Suicidal Behavior Disorder, Nonsuicidal Self-Injury, Persistent Complex Bereavement Disorder, GI Tract Related Diseases (e.g., IBS), Epilepsy, Sickle Cell Disease, locked-in syndrome, restless leg syndrome, stroke (such as ischemic stroke or hemorrhagic stroke), or Amyotrophic Lateral Sclerosis (ALS).

In some embodiments, the disclosure provides a method of treating a subject, the method comprising administering to the subject a therapeutically-effective dose of psilocybin, wherein after administration the subject exhibits an improvement in cognition. In some embodiments, the improvement in cognition is an improvement in attention, episodic memory, working memory, spatial memory, social cognition, executive function, and/or cognitive flexibility.

In some embodiments, the disclosure provides a method of treating a subject in need thereof, the method comprising administering to the subject a therapeutically-effective dose of psilocybin, wherein the subject has Treatment Resistant Depression (TRD).

In some embodiments, the disclosure provides a method of treating a subject in need thereof, the method comprising administering to the subject a therapeutically-effective dose of psilocybin, wherein the subject has Major Depressive Disorder (MDD).

### Pre-Treatments and Combination Therapies

In some embodiments, the methods of treatment comprising administering psilocybin to a subject in need thereof further comprise pretreating the subject with magnesium before administration of the psilocybin. Sometimes, magnesium is administered daily for a least 1 day, at least 2 days, at least 3 days, at least 4 days, at least 5 days, at least 6 days, at least 1 week, at least 2 weeks, at least 3 weeks, at least 4 weeks, at least 5 weeks, or at least 6 weeks before administration of the psilocybin. In some embodiments, about 10 mg to about 500 mg of magnesium are administered to the subject per day. In some embodiments, about 30 mg, about 75 mg, about 80 mg, about 130 mg, about 240 mg, about 310 mg, about 320 mg, about 360 mg, about 410 mg, about 400 mg, or about 420 mg are administered to the subject per day. In some embodiments the magnesium is administered to the subject on the same day as the psilocybin. In some embodiments, the magnesium is administered to the subject immediately before, concurrently with, or immediately after administration of the psilocybin. In some embodiments, magnesium supplements are administered to the subject until the subject's blood level for magnesium is about 1.5 to about 2.5 mEq/L. In some embodiments, psilocybin is not administered to the subject if the subject's blood level of magnesium is less than about 1.5 to about 2.5 mEq/L.

In some embodiments, the methods of treatment comprising administering psilocybin to a subject in need thereof further comprise pretreating the subject with niacin before administration of the psilocybin. Sometimes, niacin is administered daily for a least 1 day, at least 2 days, at least 3 days, at least 4 days, at least 5 days, at least 6 days, at least 1 week, at least 2 weeks, at least 3 weeks, at least 4 weeks, at least 5 weeks, or at least 6 weeks before administration of the psilocybin. In some embodiments, about 1 mg to about 5,000 mg of niacin are administered to the subject per day, for example about 1 mg to about 50 mg, about 10 mg to about 100 mg, about 100 mg to about 200 mg, about 1 mg to about 200 mg, about 100 mg to about 200 mg, about 10 mg to about 50 mg, about 10 to about 35 mg, about 100 mg to about 500 mg, or about 1,000 mg to about 3,000 mg. In some embodiments, about 10 mg, about 14 mg, about 15 mg, about 16 mg, about 20 mg, about 30 mg, about 35 mg, about 50 mg, about 60 mg, about 75 mg, about 100 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1500 mg, about 2000 mg, about 2500 mg, or about 3000 mg of niacin are administered to the subject per day (while avoiding any toxic exposure from excess niacin). In some embodiments, niacin is included as an ingredient/component, for example, to reduce risk of abuse and/or to improve efficacy. In some embodiments the niacin is administered to the subject on the same day as the psilocybin. In some embodiments, the niacin is administered to the subject immediately before, concurrently with, or immediately after administration of the psilocybin.

In some embodiments, psilocybin is administered to the subject in combination with one or more additional therapies. In some embodiments, psilocybin is administered to the subject in combination with one or more anti-depressant or anti-anxiety drugs, such as SSRIs, tricyclic antidepressants (TCAs), monoamine oxidase inhibitors (MAOIs), or serotonin norepinephrine reuptake inhibitors (SNRIs).

In some embodiments, psilocybin is administered to the subject in combination with an SSRI. In some embodiments, psilocybin is administered to the subject in combination with escitalopram, sertraline, fluoxetine, vilazodone, paroxetine or citalopram. In some embodiments, psilocybin is administered to the subject in combination with escitalopram, sertraline, fluoxetine, paroxetine, citalopram, vilazadone or vortioxetine.

In some embodiments, about 25 mg of psilocybin is administered to the subject in combination with an SSRI. In some embodiments, about 25 mg of psilocybin is administered to the subject in combination with escitalopram, sertraline, fluoxetine, vilazodone, paroxetine or citalopram. In some embodiments, about 25 mg of psilocybin is administered to the subject in combination with escitalopram, sertraline, fluoxetine, paroxetine, citalopram, vilazadone or vortioxetine.

In some embodiments, the disclosure provides a method of reducing anxiety in a subject undergoing treatment with psilocybin, the method comprising administering to the subject: i) psilocybin or a precursor or derivative thereof, and ii) one or more benzodiazepines.

In some embodiments, the one or more benzodiazepines are administered to the subject at or around the same time as the psilocybin or precursor or derivative thereof. In some embodiments, the one or more benzodiazepines are administered to the subject prior to administration of the psilocybin or precursor or derivative thereof, such as about 10 minutes, about 15 minutes, about 20 minutes, about 30 minutes, about 45 minutes, about 60 minutes, about 75 minutes, about 90 minutes, about 105 minutes, about 120 minutes, about 150 minutes, or about 180 minutes before administration of the psilocybin or precursor or derivative thereof. In some embodiments, the one or more benzodiazepines are administered to the subject after the psilocybin or precursor or derivative thereof, such as about 10 minutes, about 15 minutes, about 20 minutes, about 30 minutes, about 45 minutes, about 60 minutes, about 75 minutes, about 90 minutes, about 105 minutes, about 120 minutes, about 150 minutes, or about 180 minutes after administration of the psilocybin or precursor or derivative thereof.

In some embodiments, the one or more benzodiazepines are administered at a dose that is lower than doses typically used to treat anxiety, such as about 10%, 20%, 25%, 30%, 40%, 50%, or 75% of a typical dose. In some embodiments, the one or more benzodiazepines are administered at a dose that is approximately equivalent to doses typically used to treat anxiety. In some embodiments, the one or more benzodiazepines are administered at a dose that is higher than doses typically used to treat anxiety, such as about 125%, 150%, 175%, 200%, 250%, or 300% of a typical dose. In some embodiments, the one or more benzodiazepine is administered orally to the subject.

In some embodiments, the benzodiazepine is selected from the group consisting of adinazolam, alprazolam, bentazepam, bretazenil, bromazepam, bromazolam, brotizolam, camazepam, chlordiazepoxide, cinazepam, cinolazepam, clobazam, clonazepam, clonazolam, clorazepate, clotiazepam, cloxazolam, delorazepam, deschloroetizolam, diazepam, diclazepam, estazolam, ethyl carfluzepate, ethyl loflazepate, etizolam, flualprazolam, flubromazepam, flubromazolam, fluclotizolam, flunitrazepam, flunitrazolam, flurazepam, flutazolam, flutoprazepam, halazepam, ketazolam, loprazolam, lorazepam, lormetazepam, meclonazepam, medazepam, metizolam, mexazolam, midazolam, nifoxipam, nimetazepam, nitemazepam, nitrazepam, nitrazolam, nordiazepam, norflurazepam, oxazepam, phenazepam, pinazepam, prazepam, premazepam, pyrazolam, quazepam, rilmazafone, temazepam, tetrazepam, and triazolam.

In certain embodiments, a patient is administered psilocybin or a precursor or derivative thereof as described herein along with one or more 5-HT_{2A} specific antagonists and/or inverse agonists. In some embodiments, the patient is administered psilocybin or a precursor or derivative thereof and the one or more 5-HT_{2A} specific antagonists and/or inverse agonists at the same time. In other embodiments, the patient is administered one or more 5-HT_{2A} specific antagonists and/or inverse agonists prior to psilocybin administration, such as, but not limited to about 10 minutes, about 15 minutes, about 20 minutes, about 30 minutes, about 45 minutes, about 60 minutes, about 75 minutes, about 90 minutes, about 105 minutes, about 120 minutes, about 150 minutes, or about 180 minutes before psilocybin administration. In some embodiments, the patient is administered one or more 5-HT_{2A} specific antagonists and/or inverse agonists after psilocybin administration, such as, but not limited to about 10 minutes, about 15 minutes, about 20 minutes, about 30 minutes, about 45 minutes, about 60 minutes, about 75 minutes, about 90 minutes, about 105 minutes, about 120 minutes, about 150 minutes, or about 180 minutes after psilocybin administration.

In certain embodiments, the one or more 5-HT_{2A} specific antagonists and/or inverse agonists are administered at doses that are lower than doses typically used, e.g., about 10%, about 20%, about 25%, about 30%, about 40%, about 50%, or about 75% of a typical dose. In other embodiments, the one or more 5-HT_{2A} specific antagonists and/or inverse agonists are administered at doses that are equivalent to doses typically used. In yet other embodiments, the one or more 5-HT_{2A} specific antagonists and/or inverse agonists are administered at doses that are higher than doses typically used, e.g., about 125%, about 150%, about 175%, about 200%, about 250%, or about 300% of a typical dose.

Suitable 5-HT_{2A} antagonists include but are not limited to, trazodone, mirtazapine, metergoline, ketanserin, ritanserin, nefazodone, clozapine, olanzapine, quetiapine, risperidone, asenapine, MDL-100907, cyproheptadine, pizotifen, LY-367,265, 2-alkyl-4-aryl-tetrahydro-pyrimido-azepine, 9-aminomethyl-9,10-dihydroanthracene (AMDA), haloperidol, chlorpromazine, hydroxyzine (atarax), 5-MeO-NBpBrT, niaprazine, altanserin, aripiprazole, etoperidone, setoperone, chlorprothixene, cinaserin, adatanserin, medifoxamine, rauwolscine, phenoxybenzamine, pruvanserin, deramciclane, nelotanserin, lubazodone, mepiprazole, xylamidine, R-(+)-alpha-(2,3-dimethoxyphenyl)-1-[2-(4-fluorophenethyl)]-4-piperidinemethanol (M100907), mianserin, AT 1015, DV 7028, eplivanserin, 4F 4PP, fanaserin, alpha-phenyl-1-(2-phenylethyl)-4-piperidinemethanol (MDL 11,939), melperone, mesulergine, paliperidone, 1-[2-(3,4-Dihydro-1H-2-benzopyran-1-yl)ethyl]-4-(4-fluorophenyl) piperazine dihydrochloride (PNU 96415E), (2R,4R)-5-[2-[2-[2-(3-methoxyphenyl)ethyl]phenoxy]ethyl]-1-methyl-3-pyrrolidinol (R-96544), sarpogrelate, spiperone, ziprasidone, zotepine, and 7-[[4-[2-(4-fluorophenyl)ethyl]-1-piperazinyl]carbonyl]-1H-indole-3-carbonitrile (EMD 281014).

Suitable 5-HT_{2A} reverse agonists include but are not limited to, AC-90179, nelotanserin (APD-125), eplivanserin, pimavanserin (ACP-103), and volinaserin.

In certain embodiments, the 5-HT_{2A} antagonist is selected from the compounds of Table F.

TABLE F
5-HT2A antagonists

Acepromazine
Agomelatine
Amitriptyline
Amoxapine
Amperozide
APD791
Aripiprazole
Aripiprazole lauroxil
Blonanserin
Brexpiprazole
Butriptyline
Captodiame
Cariprazine
Chlorpromazine
Chlorprothixene
Cinitapride
Citalopram
Clomipramine
Clozapine
Cyclobenzaprine
Cyproheptadine
Deramciclane
Desipramine
Dosulepin
Doxepin
Epinastine
Esmirtazapine
Etoperidone
Flibanserin
Fluoxetine
Flupentixol
Fluspirilene
Iloperidone
Imipramine
Lisuride
Loxapine
Lurasidone
Mesoridazine
Methotrimeprazine
Methysergide
Mianserin
Mirtazapine
Nefazodone
Nortriptyline
Olanzapine
Paliperidone
Pimavanserin
Pizotifen
Promazine
Propiomazine
PRX-08066
Quetiapine
Risperidone
Sertindole
Thioproperazine
Thioridazine
Tramadol
Trazodone
Triflupromazine
Trimipramine
YKP-1358
Yohimbine
Ziprasidone
Zotepine
Zuclopenthixol

In some embodiments, the disclosure provides a method of reducing the negative side effects associated with a traumatic psychedelic experience in a patient undergoing treatment with psilocybin, the method comprising administering to the patient: i) psilocybin or a precursor or derivative thereof, and ii) one or more cannabinoids or cannabinoid derivatives.

In some embodiments, the cannabinoid is selected from the group consisting of THC (tetrahydrocannabinol), THCA (tetrahydrocannabinolic acid); CBD (cannabidiol); CBDA (cannabidiolic acid); CBN (cannabinol); CBG (cannabigerol); CBC (cannabichromene); CBL (cannabicyclol); CBV (cannabivarin); THCV (tetrahydrocannabivarin); CBDV (cannabidivarin); CBCV (cannabichromevarin); CBGV (cannabigerovarin); CBGM (cannabigerol monomethyl ether); CBE (cannabielsoin); and CBT (cannabicitran). In particular embodiments, the cannabinoid is CBD (cannabidiol).

In some embodiments, at least one symptom of a disease, disorder, or condition described herein is alleviated within 24 hours of administering psilocybin. In some embodiments, at least one symptom of the disease, disorder, or condition is alleviated within 1 week of the administering. In some embodiments, at least one symptom of the disease, disorder, or condition is alleviated within 1 month of the administering. In some embodiments, at least one symptom of the disease, disorder, or condition is alleviated within 6 months of the administering. In some embodiments, at least one symptom of the disease, disorder, or condition is alleviated within 12 months of the administering.

In some embodiments, at least one symptom of the disease, disorder, or condition is alleviated for a period of at least 1 month after administering psilocybin. In some embodiments, at least one symptom of the disease, disorder, or condition is alleviated for a period of at least 3 months after the administering. In some embodiments, at least one symptom of the disease, disorder, or condition is alleviated for a period of at least 6 months after the administering. In some embodiments, at least one symptom of the disease, disorder, or condition is alleviated for a period of at least 12 months after the administering.

In some embodiments, no other treatment is administered to the subject to treat the disease, disorder, or condition before administration of the psilocybin. In some embodiments, no other treatment is administered to the subject to treat the disease, disorder, or condition after administration of the psilocybin.

### Safety and Efficacy of Psilocybin

The present disclosure also relates to the safety and efficacy of the use of psilocybin as disclosed herein. The following is a non-exhaustive list of tests that can be used to determine the effects of psilocybin, and in particular the psilocybin formulations as disclosed herein administered as disclosed herein.

In some embodiments, the Spatial Working Memory (SWM) test is utilized to evaluate the safety and efficacy of psilocybin as disclosed herein. SWM requires retention and manipulation of visuospatial information. Study subjects are required to find the blue tokens in the on-screen ‘boxes’. Boxes are searched by touching them to determine whether they contain a token. Once a token has been located it is ‘stacked’ in a column on the right of the screen. Study subjects then search for further tokens until they have all been located. The remaining tokens will thereafter only be found in boxes that have not so far yielded a token. Study subjects are explicitly told this is the case and it they revisit a box in which a token has been found they commit a ‘between error’, the usual primary metric for this test. Occasions on which the subject revisits a box in the same search are scored as a ‘within’ error. Many study subjects will adopt a search strategy via which they systematically search the array of boxes. This is also scored by the Cambridge Neuropsychological Test Automated Battery system and yields a ‘strategy’ score. SWM performance is impaired by damage to the prefrontal cortex, especially the dorsolateral prefrontal cortex. Similarly, in neuroimaging studies in healthy volunteers, SWM performance is associated with activations in the dorsolateral and mid-ventrolateral prefrontal cortex. This test takes approximately 4 min to complete.

In some embodiments, the efficacy of psilocybin is evaluated using the spatial working memory between errors (SWMBE) score. In some embodiments, after treating according to the methods of the disclosure, a subject's SWMBE score decreases by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, or more, compared to prior to treatment.

In some embodiments, the efficacy of psilocybin is evaluated using the spatial working memory strategy (SWMS) score. In some embodiments, after treating according to the methods of the disclosure, a subject's SWMS score decreases by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, or more, compared to prior to treatment.

In some embodiments, the Rapid Visual Information Processing (RVP) test is utilized to evaluate the safety and efficacy of psilocybin. The RVP is a measure of sustained attention outputting measures of response accuracy, target sensitivity and reaction times. In this test, the study subject is required to monitor a stream of digits from 2 to 9 for specific sequences (e.g., 3-5-7) and to acknowledge detection of the sequence by touching the on-screen response button as quickly as possible after presentation of the third digit. Digits are presented pseudorandomly to create the possibility of ‘false alarm’ responses in which the first 2 digits of a sequence are not followed by a true target, e.g., when 3 is followed by a 5, but not then by a 7. In order to complete the task successfully study subjects must sustain attention to the white box in which the digits appear. Performance on this task is measured by the speed of response to the presentation of the final digit of a target, as well as the study subject's ability to detect specified sequences. This test takes approximately 7 min to complete. In some embodiments, performance on the Rapid Visual Information Processing test is reported using a RVP A Prime (RPVA) score. Higher scores on the RVPA indicated better performance. In some embodiments, after treating according to the methods of the disclosure, a subject's RVPA score increases by between about 5% and about 300%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, or about 300%, or more, compared to prior to treatment.

In some embodiments, the Paired Associates Learning (PAL) test is utilized to evaluate safety and/or efficacy of psilocybin. The PAL task is a measure of visuo-spatial memory in which study subjects are required to remember locations at which visual stimuli are located. Boxes are displayed on the screen and are “opened” in a randomized order. One or more of them will contain a pattern. The patterns are then displayed in the middle of the screen, one at a time and the subject must select the box in which the pattern was originally located. If the subject makes an error, the boxes are opened in sequence again to remind the subject of the locations of the patterns. Increased difficulty levels can be used to test high-functioning, healthy individuals. The primary metric for this test is the number of errors made. This test takes approximately 8 min to complete. Successful performance of the PAL test is dependent on functional integrity of the temporal lobe, particularly the entorhinal cortex. In some embodiments, the Paired Associates Learning total errors adjusted (PALTEA) score is used to assess the efficacy of psilocybin. In some embodiments, after treating according to the methods of the disclosure, a subject's PALTEA score decreases by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, or more, compared to prior to treatment.

In some embodiments, the efficacy and/or safety of psilocybin is evaluated using the cognitive flexibility panel test.

In some embodiments, the Emotion Recognition Task (ERT) test is utilized to evaluate the safety and/or efficacy of psilocybin. The ERT measures the ability to identify 6 basic emotions in facial expressions along a continuum of expression magnitude. In some embodiments, the ERT is performed according to the following protocol: Subjects are shown computer morphed images derived from the facial features of real individuals each showing a specific emotion, on a screen, one at a time. Each face is displayed for 200 ms and then immediately covered up, and the subject must select which emotion the face displayed from the six options (happy, sad, anger, fear, surprise, disgust). The ERT percent correct (ERTPC) of correct responses (emotion selection) the subject made is assessed. A higher score indicates better performance. In some embodiments, after treating according to the methods of the disclosure, a subject's ERTPC increases by between about 5% and about 300%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, or about 300%, or more, compared to prior to treatment.

In some embodiments, the Intra-Extra Dimensional Set Shift (IED) test is used to evaluate the safety and/or efficacy of psilocybin. The IED consists of four 7-item subscales, each of which taps a separate aspect of the global concept “empathy.” In some embodiments, the Intra-Extra Dimensional Set Shift total errors (IEDYERT) score is used to assess the efficacy of psilocybin. In some embodiments, after treating according to the methods of the disclosure, a subject's IEDYERT score decreases by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, or more, compared to prior to treatment.

In some embodiments, the One Touch Stockings (OTS) of Cambridge test is used to evaluate the safety and/or efficacy of psilocybin. The OTS is a test of executive function, based upon the Tower of Hanoi test. It assesses both the spatial planning and the working memory subdomains. This test takes approximately 10 min to perform. The OTS test reports an one touch stockings of Cambridge problems solved on first choice (OTSPSFC) score. A higher OTSPSFC score is associated with better executive function. In some embodiments, after treatment according to the methods of the disclosure, a subject's OTSPSFC score increases by between about 5% and about 300%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, or about 300%, or more, as compared to prior to treatment.

In some embodiments, verbal fluency is used to evaluate the safety and/or efficacy of psilocybin. In the verbal fluency test, the study subject is asked to name as many different category exemplars (e.g., ‘animals’) as they can in 1 min, subject to certain scoring rules, such as repetition. Successful performance on this test is reliant on the integrity of a number of cognitive abilities and especially those traditionally viewed as executive functions, such as planning and working memory. The primary metric for this test is the total number of acceptable words generated. In some embodiments, after treatment with psilocybin, a subject's verbal fluency category score improves by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, or about 300%, or more, as compared to prior to treatment.

In some embodiments, the Digit Span Forward (DSF) test is used to evaluate the safety and/or efficacy of psilocybin. DSF is used to measure number storage capacity. Subjects hear a sequence of digits and are tasked to recall the sequence correctly, with increasingly longer sequences being tested in each trial. The subject's span is the longest number of sequential digits that can accurately be remembered. Digit span tasks can be given forwards or backwards, meaning that once the sequence is presented, the subject is asked to either recall the sequence in normal or reverse order. For this study, subjects will be asked to recall the sequence in the order presented, i.e., Digit Span Forward. The primary metric for this test is the number of digit sequences successfully recalled. In some embodiments, after treatment with psilocybin, a subject's Digit Span Forward score improves by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, or about 300%, or more, as compared to prior to treatment.

In some embodiments, the Five Dimension Altered States of Consciousness questionnaire (5D-ASC) is utilized to evaluate the safety and/or efficacy of psilocybin. The 5D-ASC measures the acute drug effects using 5 primary dimensions and 11 lower-order scales to assess alterations in mood, perception and experience of self in relation to environment and thought disorder. The 5 dimensions include oceanic boundlessness, anxious ego dissolution, visionary restructuralization, auditory alterations and reduction of vigilance. In some embodiments, after treatment according to the methods of the disclosure, a subject experiences an increase on a dimension or a subscale compared to prior to treatment. The lower-order scales include “experience of unity,” “spiritual experience,” “blissful state,” “insightfulness,” “disembodiment,” “impaired control of cognition,” “anxiety,” “complex imagery,” “elementary imagery,” “audio-visual synesthesia,” and “changed meaning of percepts.” In some embodiments, the increase is about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, or about 300%, or more, compared to prior to treatment.

In some embodiments, the Positive and Negative Affect Schedule (PANAS) is used to evaluate the safety and/or efficacy of psilocybin. The PANAS measures the acute emotional drug effects and comprises 2 mood scales that measure positive and negative affect. Positive affect refers to the propensity to experience positive emotions and interact with others positively. Negative affect involves experiencing the world in a more negative way. Subjects respond to 10 questions associated with negative affect and 10 questions associated with positive affect. The questions are scaled using a 5-point scale that ranges from “slightly or not at all (1)” to “extremely (5)”. A total higher score on the positive affect questions indicates more of a positive effect while a lower score on the negative affect questions indicates less of a negative affect. In some embodiments, after treating according to the methods of the disclosure, a subject experiences a decrease in negative affect score of the PANAS, between about 5% and about 100%, for example about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, compared to prior to treatment. In some embodiments, after treating according to the methods of the disclosure, a subject experiences an increase in positive affect score of the PANAS, between about 5% and about 100%, for example about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, or about 300%, or more, compared to prior to treatment.

In some embodiments, the NEO-Five Factor Inventory (NEO-FFI) test is used to evaluate the safety and/or efficacy of psilocybin. The NEO-FFI evaluates 5 broad domains of personality—Neuroticism, Extroversion, Openness, Agreeableness and Conscientiousness.

In some embodiments, the Symptom Checklist-90 item (SCL-90) questionnaire is used to evaluate the safety and/or efficacy of psilocybin. The SCL-90 is a relatively brief self-report psychometric instrument designed to evaluate a broad range of psychological problems and symptoms of psychopathology. In some embodiments, the SCL-90 is used to assess somatization, obsessive-compulsive behaviors, interpersonal sensitivity, depression, anxiety, hostility, phobic anxiety, paranoid ideation, and psychoticism of a subject treated according to the methods of the disclosure. The 90 items in the questionnaire are scored on a 5-point Likert scale, indicating the rate of occurrence of the symptom during the time reference. In some embodiments, after treating according to the methods of the disclosure, a subject's SCL-90 score decreases by about 5% to about 100%, for example, by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

In some embodiments, the Life Changes Inventory (LCI) questionnaire is utilized to evaluate the safety and/or efficacy of psilocybin. The LCI is designed as a questionnaire to investigate those variables present in the day-to-day experience of adults that might relate either to stability or decline of intellectual ability.

In some embodiments, Social Cognition Panel scales are utilized to evaluate the safety and/or efficacy of psilocybin. The social cognition panel scales comprise the pictorial empathy test (PET), reading the mind in the eyes test (RMET), social value orientation (SVO) test, the Toronto Empathy Questionnaire (TEQ), and the scale of social responsibility (SSR).

In some embodiments, the Pictorial Empathy Test (PET) is utilized to evaluate the effect of psilocybin on affective empathy.

In some embodiments, Reading the Mind in the Eyes Test (RMET) is utilized to evaluate the safety and/or efficacy of psilocybin. The RMET has 36 items, in which subjects are presented with a photograph of the eyes region of the face and must choose 1 of 4 adjectives or phrases to describe the mental state of the person pictured. A definition handout is provided at the beginning of the task and a practice item precedes the first trial.

In some embodiments, the Social Value Orientation (SVO) test is utilized to evaluate the safety and/or efficacy of psilocybin. The SVO Slider Measure has 6 primary items with 9 secondary (and optional) items. All of the items have the same general form. Each item is a resource allocation choice over a well-defined continuum of joint payoffs.

In some embodiments, after treating according to the methods of the disclosure, one or more of the subject's Social Cognition Panel Scales Score, i.e., PET, RMET, SVO, TEQ, and/or SSR score), improves by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, about 110%, about 120%, about 130%, about 140%, about 150%, about 160%, about 170%, about 180% about 190%, about 200%, about 210%, about 220%, about 230%, about 240%, about 250%, about 260%, about 270%, about 280%, about 290%, or about 300%, or more, compared to prior to treatment.

In some embodiments, the Toronto Empathy Questionnaire (TEQ) is utilized to evaluate the safety and/or efficacy of psilocybin. The TEQ represents empathy as a primarily emotional process. The TEQ has exhibited good internal consistency and high test-retest reliability. The TEQ is a brief, reliable and valid instrument for the assessment of empathy. In some embodiments, after treating according to the methods of the disclosure, a subject's TEQ score increases by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, or about 300%, or more, compared to prior to treatment.

In some embodiments, the Scale of Social Responsibility (SSR) is utilized to evaluate the safety and/or efficacy of psilocybin. The SSR measures perceptions regarding the importance of ethics and social responsibility.

In some embodiments, the Sheehan Suicidality Tracking Scale (SSTS) is utilized to evaluate the safety and/or efficacy of psilocybin. The SSTS is a 16-item scale that assesses the seriousness of suicidality phenomena on a Likert-type scale (0-4) ranging from “not at all” (0) to “extremely”. The SSTS assesses the frequency of key phenomena and the overall time spent in suicidality.

In some embodiments, the Mini International Neuropsychiatric Interview (MINI) (version 7.0.2) is utilized to evaluate the safety and efficacy of psilocybin. The MINI is a brief structured interview for the major Axis I psychiatric disorders in DSM-5 and International Classification of Diseases-10. In some embodiments, the MINI is used to diagnose a subject with a disorder.

In some embodiments, the McLean Screening Instrument for Borderline Personality Disorder (MSIBPD) is utilized for evaluating the safety and/or efficacy of psilocybin. The MSIBPD is a useful screening tool for identifying the presence of DMS-IV borderline personality disorder.

In some embodiments, the Tellegen Absorption Scale is utilized for evaluating the safety and/or efficacy of psilocybin. The Tellegen Absorption Scale is a 34-item multidimensional measure that assesses imaginative involvement and the tendency to become mentally absorbed in everyday activities.

In some embodiments, the safety and/or efficacy of psilocybin is evaluated by physical examination. A physical examination, includes, but is not limited to, an examination of the subject's general appearance, including an examination of the skin, neck, eyes, ears, nose, throat, heart, lungs, abdomen, lymph nodes, extremities and musculoskeletal system.

In some embodiments, body weight and height of a subject are assessed. In some embodiments, body mass index is used to assess the safety and/or efficacy of psilocybin.

In some embodiments, an electrocardiogram (ECG) is utilized to evaluate the safety and/or efficacy of psilocybin. In some embodiments, a Standard 12-lead ECG is obtained.

In some embodiments, vital signs of a subject are used to evaluate safety and/or efficacy of psilocybin. Vital signs include, but are not limited to, blood pressure (BP), respiratory rate, oral body temperature and pulse. In some embodiments, blood pressure is taken after a subject has been sitting down for at least three minutes.

In some embodiments, clinical laboratory tests are utilized to evaluate the safety and/or efficacy of psilocybin. In some embodiments, the clinical laboratory tests include blood samples and/or urine samples. In some embodiments, hemoglobin, hematocrit, red blood cell count, mean corpuscular hemoglobin, mean corpuscular volume, mean corpuscular hemoglobin concentration, white blood cell count (with differential) and platelet count are measured to evaluate safety and/or efficacy of psilocybin. In some embodiments, albumin, alkaline phosphatase, alanine aminotransferase (ALT), amylase, aspartate aminotransferase (AST), bicarbonate, bilirubin (direct, indirect and total), calcium, chloride, creatine kinase, creatinine, γ-glutamyl transferase, glucose, lactate dehydrogenase, lipase, magnesium, phosphate, potassium, protein-total, sodium, blood urea nitrogen and/or uric acid are measured to evaluate the safety and/or efficacy of psilocybin.

In some embodiments, urine is tested for pregnancy and/or illicit drugs.

In some embodiments, the safety and/or efficacy of psilocybin are evaluated by measuring adverse events. Adverse events are classified as mild, moderate, or severe. A mild adverse event does not interfere in a significant manner with the subject's normal level of functioning. A moderate adverse event produces some impairment of functioning, but is not hazardous to the subject's health. A serious adverse event produces significant impairment of functioning or incapacitation and is a definite hazard to the subject's health. Adverse events may include, for example, euphoric mood, dissociative disorder, hallucination, psychotic disorder, cognitive disorder, disturbances in attention, mood alterations, psychomotor skill impairments, inappropriate affects, overdoses, and intentional product misuse. In some embodiments, serious adverse events include death, life-threatening adverse events, inpatient hospitalization or prolongation of existing hospitalization, persistent or significant disability/incapacity, and congenital anomaly/birth defect in the offspring of a subject who received psilocybin. In some embodiments, serious adverse events include intensive treatment in an emergency room or at home for allergic bronchospasm, blood dyscrasias or convulsions that do not result in inpatient hospitalization, or the development of drug dependency or drug abuse.

### Depression Assessments

In some embodiments, the sign or symptom of depression is measured in a subject before, during, or after treatment with the methods described herein. In some embodiments, the sign or symptom of depression is measured according to a diary assessment, an assessment by clinician or caregiver, a clinical rating scale, an imaging test, or a blood of CSF test.

In some embodiments, the sign or symptom of depression in a subject is measured using a neuropsychological assessment or clinical rating scale. In some embodiments, the neuropsychological assessment or clinical rating scale is the Hamilton Depression Rating Scale, the Clinical Global Impression (CGI) Scale, the Montgomery-Asberg Depression Rating Scale (MADRS), the Beck Depression Inventory (BDI), the Zung Self-Rating Depression Scale, the Raskin Depression Rating Scale, the Inventory of Depressive Symptomatology (IDS), the Quick Inventory of Depressive Symptomatology (QIDS), the Columbia-Suicide Severity Rating Scale, or the Suicidal Ideation Attributes Scale.

In some embodiments, the sign or symptom of depression in a subject is measured using the Hamilton Depression Rating (HAM-D) scale. The HAM-D scale is a 17-item scale that measures depression severity before, during, or after treatment. The scoring is based on 17 items and generally takes 15-20 minutes to complete the interview and score the results. Eight items are scored on a 5-point scale, ranging from 0=not present to 4=severe. Nine items are scored on a 3-point scale, ranging from 0=not present to 2=severe. A score of 10-13 indicates mild depression, a score of 14-17 indicates mild to moderate depression, and a score over 17 indicates moderate to severe depression. In some embodiments, after treatment with the methods described herein, the subject's HAM-D score decreases by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, or more, compared to prior to treatment.

In some embodiments, the sign or symptom of depression in a subject is measured using the Clinical Global Impression (CGI) scale. The CGI scale is a 3-item scale that measures illness severity, global improvement or change, and therapeutic response. The CGI is rated on a 7-point scale, with the severity of illness measured using a range of responses from 1 (normal) through 7 (amongst the most severely ill subjects). In some embodiments, after treatment with the methods described herein, the subject's CGI score decreases by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, or more, compared to prior to treatment.

In some embodiments, the sign or symptom of depression in a subject is measured using the Clinical Global Impression-Severity (CGI-S) scale. In some embodiments, after treatment with the methods described herein, the subject's CGI-S score decreases by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, or more, compared to prior to treatment.

In some embodiments, the sign or symptom of depression in a subject is measured using the Clinical Global Impression-Improvement (CGI-I) scale. In some embodiments, after treatment with the methods described herein, the subject's CGI-I score decreases by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, or more, compared to prior to treatment.

In some embodiments, the sign or symptom of depression in a subject is measured using the Montgomery-Asberg Depression Rating Scale (MADRS). The MADRS scale is a 10-item scale that measures the core symptoms of depression. Nine of the items are based upon patient report, and 1 item is on the rater's observation during the rating interview. A score of 7 to 19 indicates mild depression, 20 to 34 indicates moderate depression, and over 34 indicates severe depression. MADRS items are rated on a 0-6 continuum with 0=no abnormality and 6=severe abnormality. In some embodiments, after treatment with the methods described herein, the subject's MADRS score decreases by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, or more, compared to prior to treatment.

In some embodiments, after treatment with the methods described herein, the subject's MADRS score decreases by at least about 50% compared to prior to treatment. In some embodiments, after treatment with the methods described herein, the subject's MADRS score decreases by between 5 and about 20 points, for example, about 5 points, about 6 points, about 7 points, about 8 points, about 9 points, about 10 points, about 11 points, about 12 points, about 13 points, about 14 points, about 15 points, about 16 points, about 17 points, about 18 points, about 19 points and about 20 points, compared to prior to treatment. In some embodiments, after treatment with the methods described herein, the subject's MADRS score is ≤10 points (i.e., the treated subject is a MADRS remitter). In some embodiments, the subject's MADRS score is ≤10 points for at least about 3 weeks to about 12 weeks, for example, about 3 weeks, about 4 weeks, about 5 weeks, about 6 weeks, about 7 weeks, about 8 weeks, about 9 weeks, about 10 weeks, about 11 weeks and about 12 weeks, after treatment with the methods described herein.

In some embodiments, the sign or symptom of depression in a subject is measured using the Beck Depression Inventory (BDI). The BDI is a 21-item, self-report rating inventory that measures characteristic attitudes and symptoms of depression. The items are rated on 0-3 continuum with 0=no abnormality and 3=severe abnormality. A score of 17-20 indicates borderline clinical depression, a score of 21-30 indicates moderate depression, a score of 31-40 indicates severe depression, and over 40 indicates extreme depression. In some embodiments, after treatment with the methods described herein, the subject's BDI score decreases by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, or more, compared to prior to treatment.

In some embodiments, the sign or symptom of depression in a subject is measured using the Zung Self-Rating Depression Scale. The Zung Self-Rating Depression Scale is a 20-item self-report questionnaire that measures the psychological and somatic symptoms associated with depression. The questionnaire takes about 10 minutes to complete, and items are framed in terms of positive and negative statements. Each item is scored on a Likert scale ranging from 1 to 4. A total score is derived by summing the individual item scores, and ranges from 20 to 80. Most people with depression score between 50 and 69, while a score of 70 and above indicates severe depression. In some embodiments, after treatment with the methods described herein, the subject's Zung Self-Rating Depression score decreases by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, or more, compared to prior to treatment.

In some embodiments, the sign or symptom of depression in a subject is measured using the Raskin Depression Rating Scale. The Raskin Depression Rating Scale measures baseline levels of depression and change in depression severity overtime. Each item is scored on a scale ranging from 1 to 5 with 1=not at all and 5=very much. A total score is derived by summing the individual item scores, and scores of 9 or greater represents moderate depression. In some embodiments, after treatment with the methods described herein, the subject's Raskin Depression Rating Scale score decreases by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, or more, compared to prior to treatment.

In some embodiments, the sign or symptom of depression in a subject is measured using the Inventory of Depressive Symptomatology (IDS). The IDS is a 30-item inventory that measures depressive signs and symptoms. Each item is scored on a scale of 0 to 3 with 0=absence of pathology and 3=severe pathology. A total score is derived by summing the individual item scores; scores between 26-38 indicates mild depression, scores between 39-48 indicate moderate depression, and scores 49 or greater indicate severe depression. In some embodiments, after treatment with the methods described herein, the subject's IDS score decreases by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, or more, compared to prior to treatment.

In some embodiments, the sign or symptom of depression in a subject is measured using the Quick Inventory of Depressive Symptomatology (QIDS). The QIDS is a 16-item inventory that measures depressive signs and symptoms. Each item is scored on a scale of 0 to 3 with 0=absence of pathology and 3=severe pathology. A total score is derived by summing the individual item scores; scores between 11-15 indicate moderate depression, scores between 16-20 indicate severe depression, and scores 21 or greater indicate very severe depression. In some embodiments, after treatment with the methods described herein, the subject's QIDS score decreases by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, or more, compared to prior to treatment.

In some embodiments, the sign or symptom of depression in a subject is measured using the Young Mania Rating Scale (YMRS). The YMRS is an 11-item inventory that measures manic signs and symptoms. There are 4 items that are graded on a 0 to 8 scale, ranging from 0=absent to 8=severe. The remaining 7 items are graded on a 0 to 4 scale, ranging from 0=absent to 4=severe. A total score is derived by summing the individual item scores; scores between 9-15 indicate mild mania, scores between 16-25 indicate moderate mania, and scores 26 or greater indicate severe mania. In some embodiments, after treatment with the methods described herein, the subject's YMRS score decreases by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, or more, compared to prior to treatment.

In some embodiments, the sign or symptom of depression in a subject is measured using the Columbia-Suicide Severity Rating Scale (C-SSRS). The C-SSRS measures the severity of suicidal ideation and behavior. The scale contains 10 binary questions (no=0 points and yes=1 point) and each question addresses a different component of the subject's suicidal ideation severity and behavior. A subject is considered to have suicidal ideation and/or behavior if they answer “yes” to any of the 10 questions. In some embodiments, after treatment with the methods described herein, the subject's C-SSRS score decreases by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, or more, compared to prior to treatment.

In some embodiments, the sign or symptom of depression in a subject is measured using the Suicidal Ideation Attributes Scale (SIDAS). The SIDAS measures the presence and severity of suicidal thoughts. The scale contains 5 questions measured on a 10-point scale with 0=never and 10=always. Total scores are calculated as the sum of the 5 items and range from 0 to 50. Scores of 21 or greater indicate high risk of suicidal behavior. In some embodiments, after treatment with the methods described herein, the subject's SIDAS score decreases by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, or more, compared to prior to treatment.

In some embodiments, the sign or symptom in subjects with depression is measured using a Spielberger's Trait and Anxiety Inventory, a Generalized Anxiety Disorder 7-Item Scale, a Warwick-Edinburgh Mental Wellbeing Scale, a Flourishing Scale, a Snaith Hamilton Anhedonia Pleasure Scale, a Life Orientation Test, a Meaning in Life Questionnaire, a Brief Resilience Scale, a Dysfunctional Attitudes Scale, a 44-item Big Five Inventory, a Peters 21-item Delusional Inventory, an Examination of Anomalous Self-Experience, a Ruminative Responses Scale, a White Bear Suppression Inventory, a Barrett Impulsivity Scale, a Brief Experiential Avoidance Questionnaire, a Modified Tellegen Absoprtion Questionnaire, a Scale to Assess Therapeutic Relationship, Credibility/Expectancy Questionnaire, a Connectedness to Nature Scale, a Political Perspective Questionnaire, a Social Connectedness Scale, a Bech-Rafaelsen Mania Rating Scale, a Revised Santa Clara brief compassion scale, a Gratitude Questionnaire, a Short Suggestibility Scale, a Rosenberg Self-Esteem Scale, a Universality Subscale of the Spiritual Transcendence Scale, an Oxford Questionnaire on the Emotional Side-effects of Antidepressants, a Lauks Emotional Intensity Scale, Sexual Dysfunction Questionnaire, a Brief Index of Sexual Functioning for Women, a Sexual Perceptions Questionnaire, a Barnes Akathisia Rating Scale, a Work Productivity and Activity Impairment Questionnaire, a Work and Social Adjustment Scale, a Connectedness Questionnaire, a Standard Assessment of Personality, a Positive and Negative Syndrome Scale, a Mastery Insight Scale, a Self-Reflection and Insight Scale, a Psychological Insight Scale, a Metaphysical Beliefs Questionnaire, a Spiritual Bypassing Scale, an Adverse Childhood Experience Questionnaire, a Therapeutic Music Experience Questionnaire, a Setting Questionnaire, an Absorption in Music Scale, a Psychedelic Predictor Scale, a Surrender Scale, a EuroQOL-5 Dimension-3 Level Scale, or any combinations thereof. In some embodiments, after treatment with the methods described herein, the sign or symptom of depression measured using any of the assessments listed above decreases by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, or more, compared to prior to treatment.

In some embodiments, the sign or symptom of depression in a subject is measured using an imaging test before, during, or after treatment with the methods described herein. In some embodiments, the imaging test is a CT scan. In some embodiments, the imaging test is a functional MRI scan. In some embodiments, the functional MRI scan measures the blood oxygen level-dependent (BOLD) response as an indicator of brain activity and/or functional connectivity. In some embodiments, the BOLD response is measured in the subject at resting state, in response to emotional faces, or in response to music. In some embodiments, after treatment with the methods described herein, the BOLD response in a region of the brain increases by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, or more, compared to prior to treatment. In some embodiments, after treatment with the methods described herein, the BOLD response in the amygdala increases by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, or more, compared to prior to treatment. In other embodiments, after treatment with the methods described herein, the BOLD response in a region of the brain decreases by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, or more, compared to prior to treatment. In some embodiments, after treatment with the methods described herein, the BOLD response in the amygdala decreases by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, or more, compared to prior to treatment.

In some embodiments, the sign or symptom of depression is measured using a marker for depression in the blood or cerebral spinal fluid. In some embodiments, the marker for depression is measured in the subject before, during, or after treatment with the methods or compositions described herein. In some embodiments, the marker for depression is red blood cell folate, serum folate, vitamin B12, plasma homocysteine, serum methylfolate, and/or testing for one or more of brain-derived neurotrophic factor (BDNF) Val66Met, bone morphogenetic protein rs41271330, and/or 5-HTTLPR polymorphisms. In some embodiments, the marker for depression decreases by between about 5% and about 300%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, or about 300%, or more, compared to prior to treatment. In other embodiments, the marker for depression increases by between about 5% and about 300%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, or about 300%, or more, compared to prior to treatment.

### EXAMPLES

The following examples, which are included herein for illustration purposes only, are not intended to be limiting.

### Example 1—Formulation Development

Table 1 shows the capsule formulations of psilocybin developed for clinical use. These capsule formulations were optimized to enhance flow, blend uniformity, content uniformity and dissolution as described in WIPO Patent Appln. Pub. No. 2022/207746, the entire contents of which are incorporated herein by reference.

TABLE 1
Unit Formula for Psilocybin Capsules

| Excipient | 1 mg (%) | 1 mg (g) | 5 mg (%) | 5 mg (g) | 25 mg (%) | 25 mg (g) |
|---|---|---|---|---|---|---|
| Psilocybin | 1.0 | 7.0 | 5.0 | 35.0 | 10.0 | 70.0 |
| (COM 360) | | | | | | |
| Pregelatinized | 98.0 | 686 | 94.0 | 658 | 89.0 | 623.0 |
| starch (Starcap) | | | | | | |
| Sodium Stearyl | 1.0 | 7.0 | 1.0 | 7.0 | 1.0 | 7.0 |
| Fumarate (Pruv) | | | | | | |
| Capsule, HPMC, | 1 unit | Size 2* | 1 unit | Size 2* | 1 unit | Size 0** |
| white opaque | | | | | | |
| TOTAL | 100.0 | 700.0 | 100.0 | 700.0 | 100.0 | 700.0 |

*fill weight 100 mg,
**fill weight 250 mg

### Example 2—Clinical Study Examining Psilocybin as an Adjunctive Therapy in Participants with Treatment-Resistant Depression

The effectiveness of 25 mg COMP360 as an adjunctive therapy in participants with TRD was explored.

The study was a Phase IIb, international multicentre, fixed-dose, open-label study. The study population included adult men and women, 18 years of age or older, with TRD. Participants with TRD are defined as those who meet the Diagnostic and Statistical Manual of Mental Disorders (5th Edition; DSM-5) diagnostic criteria for single or recurrent episode of major depressive disorder (MDD) without psychotic features who have failed to respond to an adequate dose and duration of 2, 3 or 4 pharmacological treatments for the current episode. The duration of the current episode must be at least 3 months but not more than 2 years.

Participants will be outpatients and will be recruited primarily through referrals from general practitioners and specialised psychiatric services.

Subjects will be assessed for their eligibility with the Mini International Neuropsychiatric Interview, Version 7.0.2 (MINI 7.0.2), the Hamilton Depression Rating Scale (17-item; HAM-D-17), the Massachusetts General Hospital-Antidepressant Treatment History Response Questionnaire (MGH-ATRQ), the C-SSRS, and McLean Screening Instrument for Borderline Personality Disorder (MSI-BPD). At the initial Screening visit (V1), the participant will also be evaluated with the Quick Inventory of Depressive Symptomatology-Self Rated-16 item (QIDS-SR-16). Additionally, a medical history, an ECG, blood tests, and vital signs will be obtained.

All subjects will be evaluated for safety at the clinic weekly for a minimum of 3 weeks prior to psilocybin administration. During these visits, the C-SSRS and any changes in medications since the previous visit will be obtained in addition to other assessments at the study clinician's discretion.

The subject will meet with a therapist for a minimum of 3 visits during screening. These are called safety sessions and will cover what to expect during the psilocybin session. The therapist and the subject will review psychoeducational materials provided at the time of enrolment.

The day before the psilocybin session, the subjects will undergo a baseline assessment (3 to 6 weeks after initial screening [Visit 1]) that will consist of the HAM-D-17, MADRS, CGI-S and CGI-I, QIDS-SR-16, C-SSRS, GAD-7, EQ-5D-3L (administered to both subject and caregiver [the latter is not mandatory]), vital signs, urinalysis, urine drug screen, and urine pregnancy test (only for women of childbearing potential). After baseline data is entered into EDC, the CAT team will complete a final review to ensure the subject's continued eligibility. Subjects cannot be progressed to Visit 3 until this approval is received.

The psilocybin administration session (V3, day 1) will last approximately 6 h and will be supported by a trained therapist. The study drug will be administered simultaneously to up to six participants. Blood pressure will be monitored continuously during the session. The COMP360 administration session may be video recorded for training and adherence monitoring. After the acute effects of psilocybin pass, participants will be evaluated for safety and accompanied home. On day 2 (V4), the day following psilocybin administration, participants will be seen in person for a safety check, assessment of suicidality and clinical and self-report assessments, and to discuss their experience during the psilocybin administration session. All sessions between the therapist and the participant may be audio recorded for adherence monitoring and quality assurance.

Subjects will be seen at the clinic for Screening (plus 3 safety visits), baseline (day-1), day 1 (dosing), day 2, week 1, week 2 and week 3. AEs, serious adverse events (SAEs) and concomitant medication and therapies will be recorded at all clinic visits. The MADRS will be done by telephone.

Clinicians are advised to continue the prescription of the participant's baseline selective serotonin reuptake inhibitor (SSRI) (fluoxetine, fluvoxamine, sertraline, paroxetine, citalopram, vilazodone, vortioxetine, or escitalopram) for the duration of the follow-up period, ideally at the same dose.

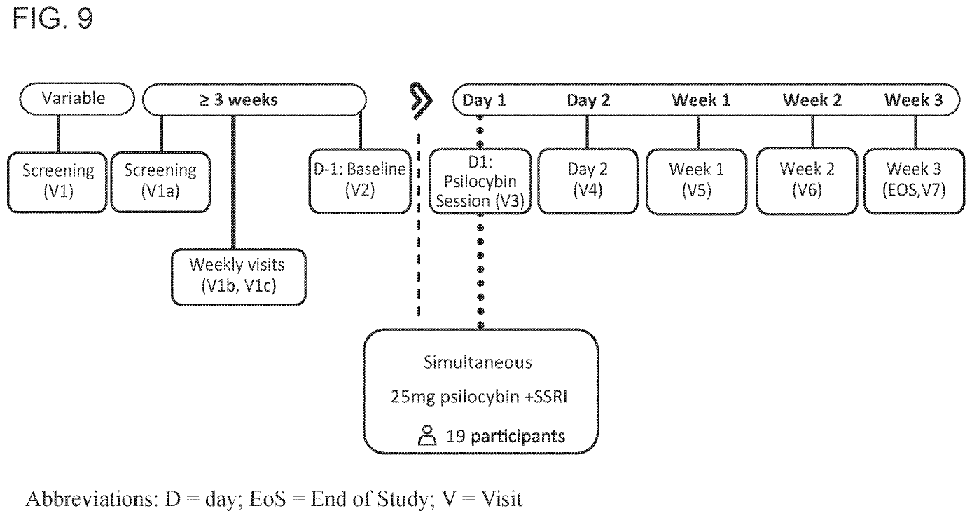

The study schematic is shown in FIG. 9 and the schedule of assessments are presented in Table 2A below:

TABLE 2A
Schedule of Assessments

| | 3 weeks prior | | | Time Post Psilocybin Session | | | |
|---|---|---|---|---|---|---|---|
| | to Baseline | | Psilocybin | | | | Week 3 |
| | Screening | Baseline | Session | | Week 1 | Week 2 | Day 21 |
| Screening | Period 1 | (Day −1) | (Day 0) | Day 1 | Day 7 | Day 14 | (EOS/ET) |
| Visit | | | | | | | |
| 1 | 1a, 1b, 1c | 2 | 3 | 4 | 5 | 6 | 7 |
| Allowable Window | | | | | | | |
| — | Weekly | | +≤7 days | None | ±1 day | ±1 day | ±1 day |

Clinical Assessments and Procedures

| | | | | | | | | |
|---|---|---|---|---|---|---|---|---|
| Informed consent | ✓ | | | | | | | |
| Medical history | ✓ | | ✓ | | | | | |
| Inclusion/Exclusion | ✓ | | ✓ | | | | | |
| Criteria | | | | | | | | |
| MINI 7.0.2 | ✓ | | | | | | | |
| HAM-D-17 | ✓ | | | | | | | |
| MGH-ATRQ | ✓ | | | | | | | |
| C-SSRS₂ | ✓ | ✓ | ✓ | ✓ | ✓ | ✓ | ✓ | ✓ |
| Vital signs | ✓ | | ✓ | ✓ | ✓ | | | |
| Physical examination, | ✓ | | | | | | | |
| including weight | | | | | | | | |
| and height | | | | | | | | |
| 12-Lead ECG | ✓ | | | | ✓ | | | |
| Clinical laboratory tests₃ | ✓ | | | | ✓ | | | ✓ |
| Urinalysis₃ | ✓ | | ✓ | | ✓ | | | |
| Urine drug screens₃ | ✓ | | ✓ | | | | | |
| Urine pregnancy test₄ | ✓ | | ✓ | | | | | |
| Documentation of | ✓ | | | | | | | |
| contraceptive | | | | | | | | |
| method to be used | | | | | | | | |
| CGI-I | | | | | ✓ | ✓ | ✓ | ✓ |
| CGI-S | | | ✓ | | ✓ | ✓ | ✓ | ✓ |
| Psilocybin dose | | | | ✓ | | | | |
| Prior/Concomitant | ✓ | ✓ | ✓ | ✓ | ✓ | ✓ | ✓ | ✓ |
| Medications | | | | | | | | |
| AE/SAEs | ✓ | ✓ | ✓ | ✓ | ✓ | ✓ | ✓ | ✓ |

Participant Completed Assessments

| | | | | | | | |
|---|---|---|---|---|---|---|---|
| MSI-BPD | ✓ | | | | | | |
| QIDS-SR-16 | ✓ | ✓ | ✓ | ✓ | ✓ | ✓ | ✓ |
| EQ-5D-3L | | ✓ | | | ✓ | ✓ | ✓ |
| GAD-7 | | ✓ | | ✓ | ✓ | ✓ | ✓ |
| PANAS | | ✓ | | ✓ | | | |
| 5D-ASC | | | ✓₆ | | | | |

Remote-rater Assessment

| | | | | | |
|---|---|---|---|---|---|
| MADRS | ✓ | ✓ | ✓ | ✓ | ✓ |

Abbreviations: 5D-ASC = Five Dimension Altered States of Consciousness; AE = adverse event; CGI-I = Clinical Global Impression - Improvement; CGI-S = Clinical Global Impression - Severity; C-SSRS = Columbia-Suicide Severity Rating Scale; ECG = electrocardiogram; EOS = End of Study; EQ-5D-3L = Euro QoL-5 dimension-3 level; ET = early termination; GAD-7 = Generalised Anxiety Disorder 7-item Scale; HAM-D-17 = Hamilton Depression Rating Scale (17-item); MINI 7.0.2 = Mini International Neuropsychiatric Interview, Version 7.0.2; MADRS = Montgomery-Asberg Depression Scale; ; MSI-BPD = McLean Screening Instrument for Borderline Personality Disorder; PANAS = Positive and Negative Affect Schedule; QIDS-SR-16 = Quick Inventory of Depressive Symptomatology - Self Rated - 16 item; SAE = serious adverse event.
¹ Screening (V1a) will be performed 3 weeks prior to the Baseline visit (V2, Day −1).
₂The “Last 12 months” version will be administered at Screening and the “Since Last Visit” version will be administered at all other visits.
₃See Section 7.2.4 of the study protocol for complete list of required tests to be performed.
₄For women of child-bearing potential only.
⁵ For females of childbearing potential and all males; site is to document method of contraception agreed to be used by each participant.
₆To be administered immediately after the COMP360 administration session.
⁷ On site clinic visits; visits allowed remotely will have the MADRS performed by telephone.

### Study Objectives

The primary objective of this study is to evaluate the efficacy of 25 mg of psilocybin adjunct to serotonergic antidepressants in TRD participants, as assessed by the change in the Montgomery-Asberg Depression Rating Scale (MADRS) total score from baseline. Baseline is defined as the assessment score obtained on Day −1. The primary time point is week 3.

Additional objectives of this study are to assess the efficacy of psilocybin compared to 1 mg psilocybin on the:
(a) proportion of subjects with response defined as a ≥50% decrease in MADRS total score from baseline to week 3.
(b) proportion of subjects with remission (defined as a MADRS total score ≤10) at week 3 post psilocybin administration.
(c) changes from baseline Clinical Global Impression-Severity scale (CGI-S) at week 3 post psilocybin administration.

Another objective is to evaluate the safety and tolerability of psilocybin in subjects with TRD based on AEs, changes in vital signs, and suicidal ideation/behaviour (measured using the Columbia-Suicide Severity Rating Scale [C-SSRS]) score at all visits.

Other objectives are to evaluate the effects of psilocybin on quality of life and wellbeing, functioning and associated disability, cognitive function, and anxiety. The study endpoints are listed in Table 2B below.

TABLE 2B
Study Endpoints

| Study Endpoint | Assessments |
|---|---|
| Primary | MADRS total score from baseline (day −1) to 3 weeks post-psilocybin |
| Secondary | a) The proportion of subjects with a response (defined as a ≥50% |
| | improvement in MADRS total score from Baseline) at week 3 after the |
| | psilocybin session. |
| | b) The proportion of subjects with remission (defined as a MADRS |
| | total score ≤10) at week 3 post psilocybin. |
| | c) Changes from baseline Clinical Global Impression - Severity scale |
| | (CGI-S) at week 3 post psilocybin administration. |
| Exploratory | Change from baseline to Week 3 in the following: |
| | a) Subject EQ-5D-3L |
| | b) Caregiver EQ-5D-3L (this assessment is not mandatory); |
| | c) GAD-7; |
| | d) QIDS-SR-16; |
| | e) Clinical Global Impression - Improvement scale (CGI-I) |
| | f) Change from baseline to day 2 in Positive and Negative |
| | Affect Schedule (PANAS) |
| | g) Summary of 5-Dimensional Altered States of Consciousness |
| | questionnaire (5D-ASC) on the day of the psilocybin |
| | administration session (day 1) |

### Study Procedures by Time Point

The study timeline includes 7 visits over a period of 3 weeks to assess the effects of psilocybin as an adjunctive treatment for TRD. The screening assessments for each Visit are listed in Table 2A.

### Visit 1: Screening Period

The subject will be screened to evaluate suitability for the study. All subjects will be seen at the clinic weekly for a minimum of 3 weeks prior to baseline (Visit 2) to ensure safe discontinuation of current antidepressant therapy required by the protocol, and to conduct psychoeducation.

At the screening visit, a number of assessments shown in Table 2A will be performed and recorded.

If the subject is deemed eligible, they can proceed to the next visit (Visit 1a). Subjects cannot begin psychoeducation until they attend the clinic for the screening visit (Visit 1a).

At subsequent screening period visits (Via, V1b, etc.), medications taken and any changes in medications since the previous visit, and the C-SSRS will be obtained.

### Visit 2: Baseline Visit—Day−1

At the baseline visit, the subject's eligibility will be confirmed by reviewing the Inclusion/Exclusion criteria and updating the medical history. The baseline visit should occur the day before the anticipated psilocybin session. The following assessments performed at the baseline visit are shown in Table 2A.

If the subject continues to meet the eligibility criteria, arrange for the participant to return to the study site for investigational product (IP) administration the next day. The research team will complete a final review to ensure the subject's continued eligibility. Subjects cannot be progressed to Visit 3 until this approval is received.

### Visit 3: Psilocybin Session—Day 0

The psilocybin session (Visit 3) should occur the day after the baseline visit (Visit 2). In exceptional circumstances the subject may visit the clinic ≤7 d following the baseline visit (Visit 2). A preparation session with the therapist is always conducted the day before the psilocybin session, even if the psilocybin session is not conducted the day after baseline (Visit 2). If the subject is out of the ≤7-day window, all baseline assessments are to be repeated, except randomization. The assessments provided at the psilocybin session are listed in Table 2A.

### Visit 4: Post-Dosing Day 1

The day after treatment the subject will return to the study site for a safety check and to discuss their experience during the psilocybin administration session. The assessments provided at post-dosing session are listed in Table 2A.

### Visit 5:1 Week Post-Dosing

The subject will visit the clinic 1 week (7 days±1 day) following psilocybin administration. The assessments obtained during Visit 5 are shown in Table 2A.

### Visit 6:2 Weeks Post-Dosing

The subjects will visit the clinic 2 weeks (14 days±1 day) following psilocybin administration. The assessments obtained during Visit 6 are shown in Table 2A.

### Visit 7:3 Weeks Post-Dosing

The subjects will visit the clinic 3 weeks (21 days±1 day) following psilocybin administration for the End of Study visit. this visit is also to be completed if the participant is discontinued from the study early. The assessments obtained during Visit 7 are shown in Table 2A.

### Description of Study Procedures

Efficacy, safety, and other types of assessments performed during the study are described in Tables 2C-2E below. All measures below will be captured electronically.

TABLE 2C
Efficacy Assessments

| | |
|---|---|
| Montgomery-Asberg | MADRS evaluations will be performed by an independent |
| Depression Rating Scale | remote rater at Baseline (Day −1), Day 1 and Weeks 1, 2 and 3 |
| (MADRS) | (V2, V4, V5, V6 and V7, respectively). The MADRS is a clinician- |
| | rated scale measuring depression severity, consisting of 10 |
| | items, each scored from 0 (normal) to 6 (severe), for a total |
| | possible score of 60; higher scores denote greater severity. The |
| | structure of the telephone-based interview will be controlled |
| | through the use of The Structured Interview Guide for the |
| | MADRS (SIGMA), which provides structured probes to ensure |
| | standardisation of administration and comprehensive coverage |
| | of 10 questions. |
| Clinical Global Impression - | The CGI-S is a 7-point scale that measures the severity |
| Severity Scale | of symptoms in patients with mental disorders. It requires the |
| | clinician to rate the severity of the patient's illness at the time of |
| | assessment, relative to the clinician's past experience with |
| | patients who have the same diagnosis. Ratings range from |
| | “normal, not at all ill” (1) to “among the most extremely ill |
| | patients” (7). This will be obtained at Baseline (Day −1), Day 1, |
| | and Weeks 1, 2 and 3 (V2, V4, V5, V6 and V7, respectively). |
| Clinical Global Impression - | The CGI-I is a 7-point scale that requires the clinician to |
| Improvement Scale | rate how much the patient's symptoms have improved relative to |
| | a baseline state. Ratings range from “very much improved” (1) to |
| | “very much worse” (7). This will be obtained at Day 1 and Weeks |
| | 1, 2 and 3 (V4, V5, V6, and V7, respectively). |
| Quick Inventory of | The 16-item QIDS is a self-rated scale designed to |
| Depressive | assess the severity of depressive symptoms in the nine |
| Symptomatology - Self | diagnostic symptom domains of a major depressive episodes, |
| Rated - | exclusive of atypical or melancholic symptoms. The QIDS-SR- |
| 16 Item | 16 is sensitive to change with various treatments, demonstrating |
| | its utility in research settings. The total score ranges from 0 to 27 |
| | with 0 representing no depression and 27 representing severe |
| | depression. The total score is the sum of the nine symptom |
| | domains. The QIDS-SR-16 will be collected at every clinic visit or |
| | contact with the participant. |
| Generalized Anxiety | The GAD-7 is useful in primary care and mental health |
| Disorder Scale | settings as a screening tool and symptom severity measure for |
| | the seven most common anxiety disorders. 32 Subjects choose |
| | one of 4 severity scores associated problems related to the |
| | common anxiety disorders and then indicate the degree to which |
| | these problems caused functional and/or social difficulties. |
| | Scores are determined by calculating the values for each |
| | column. A total score is obtained by the sum of all total column |
| | values. The GAD-7 will be obtained using the electronic patient- |
| | reported outcome (ePRO) device at Baseline (Day −1), Day 1 |
| | and Weeks 1, 2 and 3 (V2, V4, V5, V6 and V7, respectively). |
| EuroQoL-5 Dimension-3 | The 3-level EQ-5D version (EQ-5D-3L) was introduced by |
| Level Scale | the EuroQoL Group in 1990. The EQ-5D-3L essentially consists |
| | of 2 pages: the EQ-5D-3L descriptive system and the EQ visual |
| | analogue scale (EQ VAS). |
| | The descriptive system comprises five dimensions: |
| | mobility, self-care, usual activities, pain/discomfort and |
| | anxiety/depression. Each dimension has 3 levels: no problems, |
| | slight problems, moderate problems, severe problems and |
| | extreme problems. The subject is asked to indicate his/her health |
| | state by ticking the box next to the most appropriate statement in |
| | each of the five dimensions. This decision results in a 1-digit |
| | number that expresses the level selected for that dimension. The |
| | digits for the five dimensions can be combined into a 5-digit |
| | number that describes the subject's health state. |
| | The EQ VAS records the subject's self-rated health on a |
| | vertical VAS, where the endpoints are labelled ‘The best |
| | imaginable health state’ and ‘The worst imaginable health state’. |
| | The VAS can be used as a quantitative measure of health |
| | outcome that reflect the subject's own judgement. |
| | The EQ-5D-3L will be obtained at Baseline (Day −1) and Week 3 |
| | (V2 and V7, respectively).. Administration to a caregiver is not |
| | mandatory. |

TABLE 2D
Safety Assessments

| | |
|---|---|
| Columbia-Suicide | The C-SSRS will be used to assess suicide potential or tendency |
| Severity Rating | as a study entry criteria and monitored throughout the study. |
| Scale (C-SSRS) | The C-SSRS is a semi-structured interview designed to assess |
| | the severity and intensity of suicidal ideation, suicidal behavior, and |
| | nonsuicidal self-injurious behavior over a specified time period. The |
| | measurement of suicidal ideation is based on 5 “yes” or “no” questions |
| | with accompanying descriptions arranged in order of increasing severity. |
| | If the patient answers “yes” to either questions 1 or 2, the intensity of |
| | ideation is assessed in 5 additional questions related to frequency, |
| | duration, controllability, deterrents, and reasons for the most severe |
| | suicidal ideation. Suicidal behavior is assessed by asking questions |
| | categorizing behaviors into actual, aborted, and interrupted attempts; |
| | preparatory behavior; and non-suicidal self-injurious behavior. |
| | If any item(s) on the C-SSRS are answered “yes”, the primary |
| | investigator or physician investigator must review the patient's |
| | responses in order to (a) at screening and Baseline determine the |
| | patient's study eligibility and potential need for referral to a mental health |
| | professional, and (b) during the study evaluate the patient's need for |
| | appropriate medical management such as a referral to a mental health |
| | professional. |
| | A significant risk of suicide is defined as a “yes” in answer to (a) |
| | questions 4 or 5 on the suicidal ideation section; or (b) any questions on |
| | any item in the suicidal behavior section. This must be reported as an |
| | SAE and followed up accordingly. Additionally, if a patient responds |
| | “yes” to any of the suicidal ideation questions 1 through 3, the |
| | investigator should apply clinical judgment to determine the need for |
| | reporting this as an AE or SAE and the need for any appropriate referral. |
| Vital Signs | Respiratory rate, blood pressure, body temperature, and pulse |
| | rate will be obtained at Screening, Baseline (Day −1), Day 0, and Day 1 |
| | (V1, V2, V3, and V4, respectively). |
| Electrocardiogram | Standard 12-lead ECGs will be obtained at screening (Visit 1) |
| | and day 1 (Visit 4). |
| Clinical Laboratory | Blood samples will be obtained at screening (Visit 1), day 1 (Visit 4), and |
| Tests | week 3 (Visit 7) for the following: |
| | a) Hematology: haemoglobin, haematocrit, red blood cell count, |
| | mean corpuscular haemoglobin, mean corpuscular volume, |
| | mean corpuscular haemoglobin concentration, white blood cell |
| | count (with differential), and platelet count. |
| | b) Chemistry: albumin, alkaline phosphatase, alanine |
| | aminotransferase (ALT), amylase, aspartate aminotransferase |
| | (AST), bicarbonate, bilirubin (direct, indirect, and total), calcium, |
| | chloride, creatine kinase, creatinine, gamma- |
| | glutamyltransferase, glucose, lactate dehydrogenase, lipase, |
| | magnesium, phosphate, potassium, protein-total, sodium, urea |
| | (blood urea nitrogen), and uric acid. |
| | Urine samples will be obtained at Screening (V1), Baseline (V2, Day −1) |
| | and Day 1 (V4) for the following: |
| | a) Urinalysis: A dipstick urinalysis will be performed for blood, |
| | glucose, ketone, protein, pH, specific gravity, nitrite, leukocytes, |
| | bilirubin, and urobilinogen. |
| | b) Urine Drug Screen: for illicit drugs or drugs of abuse at |
| | Screening (V1) and Baseline (V2, Day −1). Results of a positive |
| | drug screen will be reviewed by the study clinician for pattern of |
| | use. |
| | c) Urine Pregnancy Test: a dipstick test in women of childbearing |
| | potential at screening (Visit 1) and baseline (Visit 2). |
| Adverse Events | All AEs occurring after the participant signs the ICF and up to the |
| | last study event will be recorded. Any AEs occurring before the start of |
| | treatment (ie, before the dose of the IP on Day 0 [V3]) will be recorded in |
| | the medical history. Any AE ongoing at V7 (EOS/ET) will be followed |
| | until resolution or no longer considered clinically significant by the |
| | investigator.. |

TABLE 2E
Other Assessment Instruments

| | |
|---|---|
| Hamilton Depression | The HAM-D-17, a 17-item scale, is used to measure the degree of |
| Rating Scale (HAM-D) | symptom severity in depressed patients. The HAM-D-17 rating |
| | will be performed by the investigator using the electronic clinical |
| | outcome assessment (eCOA) device at the Screening (V1) and |
| | Baseline (V2, Day −1) only. The total score from this assessment |
| | will be used as eligibility criteria prior to treatment (minimal total |
| | symptom score ≥18). The Structured Interview Guide for the |
| | HAM-D-17 (SIGH-D) will be administered.. |
| Mini International | The MINI was designed as a brief structured interview for |
| Neuropsychiatric | the major Axis I psychiatric disorders in DSM-5 and International |
| Interview (MINI), Version | Classification of Diseases-10. Validation and reliability studies |
| 7.0.2 | have been done comparing the MINI to the Structured Clinical |
| | Interview for DMS-5 Patient Edition and the Composite |
| | International Diagnostic Interview (a structured interview |
| | developed by the World Health Organization). Version 7.0.2 of the |
| | MINI will be used for this study. The results of these studies show |
| | that the MINI has similar reliability and validity properties, but can |
| | be administered in a much shorter period (mean 18.7 ± 11.6 min, |
| | median 15 min) than the above referenced instruments. It can be |
| | used by clinicians after a brief training session. |
| | At screening (Visit 1), subjects will be assessed for MDD, |
| | as documented by DSM-5 criteria, and the lack of other |
| | psychiatric diagnoses will be confirmed by use of the MINI. |
| Massachusetts General | The MGH-ATRQ is a self-rated scale used to determine |
| Hospital-Antidepressant | treatment resistance in major depressive disorder. 5 The scale |
| Treatment History | examines the efficacy (improvement from 0%, not improved at all |
| Questionnaire (MGH- | to 100% completely improved) and adequacy of a treatment. |
| ATRQ) | Participants are asked by clinician about treatment adherence to |
| | each medication study and examines the participants' |
| | antidepressant history to identify pseudo-resistance and treatment |
| | resistance. Failure to respond to an adequate dose and duration |
| | of 2, 3, or 4 pharmacological treatments for the current episode, |
| | as determined through the MGH-ATRQ, and using the |
| | supplementary advice on additional antidepressants are not |
| | included in MGH-ATRQ. Augmentation with an add on treatment |
| | counts as a second treatment, provided it is approved for the |
| | adjunctive treatment of MDD in that country. In addition, a list of |
| | recently approved antidepressant medications not included in |
| | MGH-ATRQ will be provided to investigators. The MGH-ATRQ |
| | will be collected at Screening (V1) only. |
| McLean Screening | The MSI-BPD is a commonly used measure to assess for |
| Instrument for Borderline | BPD. The scale consists of 10 items based on the DSM-5 BPD |
| Personality Disorder | criteria; the first 8 items represent the first eight criteria in the |
| | DSM-5 for BPD diagnosis, while the last two questions assess the |
| | paranoia and dissociation criteria for BPD. Scores for the MSI- |
| | BPD range from 0 to 10, with each item rated as “1” if present and |
| | “0” if absent. A score of 7 or higher indicates a likelihood for the |
| | subject to meet criteria for BPD. The MSI-BPD will be collected at |
| | screening (Visit 1) only. |
| Five Dimension Altered | The 5D-ASC measures the acute drug effects using 5 |
| States of Consciousness | primary dimensions and respective subdimensions to assess |
| Questionnaire | alterations in mood, perception, and experience of self in relation |
| | to environment and thought disorder. The 5 dimensions include |
| | oceanic boundlessness, anxious ego dissolution, visionary |
| | restructuralization, auditory alterations, and reduction of vigilance. |
| | This will be administered immediately after the psilocybin session |
| | on Day 0 (Visit 3). |
| The Positive and | The PANAS measures the acute emotional drug effects, |
| Negative Affect Schedule | and comprises 2 mood scales that measure positive and negative |
| | affect. Subjects respond to 20 items using a 5-point scale that |
| | ranges from “slightly or not at all (1)” to “extremely (5)”. A total |
| | higher score on the positive affect questions indicates more of a |
| | positive affect while a lower score on the negative affect |
| | questions indicates less of a negative effect. This will be |
| | administered at baseline (Visit 2), the day after the psilocybin |
| | session (Visit 4), and at week 3 (Visit 7). This will be captured |
| | electronically. |

### Subject Recruitment

A total of 20 subjects with simultaneous study drug administration to up to 6 participants.

Subjects meeting all the following inclusion criteria as shown in Table 2F below should be considered for admission into the study.

TABLE 2F
Study Inclusion Criteria

| No. | Criteria |
|---|---|
| 1 | Signed consent form |
| 2 | 18 years of age or older at screening; |
| 3 | At least moderate MDD (single or recurrent episode |
| | as informed by DSM-5; if single episode, duration |
| | of ≥3 months and ≤2 years) based on medical |
| | records, clinical assessment and documented |
| | completion of the version 7.0.2 MINI |
| 4 | HAM-D-17 (17-item) score ≥18 at screening (Visit |
| | 1) and at Baseline (Visit 2) |
| 5 | Currently receiving treatment with a SSRI |
| | (fluoxetine, fluvoxamine, sertraline, paroxetine, |
| | citalopram, vilazadone, vortioxetine or escitalopram) |
| | at, or above, a minimum locally approved |
| | therapeutic dose for at least 6 weeks before |
| | Screening (V1) and Baseline (V2, Day −1). Dose |
| | changes within the adequate range are acceptable. |
| | To be defined as an adequate study, adherence of |
| | at least 75% is needed based on participant |
| | estimate of percentage of doses that were taken. |
| 6 | Failure to respond to an adequate dose and |
| | duration of 2, 3, or 4 pharmacological treatment for |
| | the current episode as determined through the |
| | MGH-ATRQ and using the supplementary advice on |
| | additional antidepressants not included in MGH- |
| | ATRQ. Augmentation with an add on treatment |
| | counts as a second treatment, provided it is |
| | approved for the adjunctive treatment of MDD in |
| | that country. |
| 7 | McLean Screening Instrument for Borderline |
| | Personality Disorder <7 at screening (Visit 1) |
| 8 | Ability to complete all protocol required assessment |
| | tools without any assistance or alteration to the |
| | copyrighted assessments, and to comply with all |
| | study visits |

Subjects meeting any of the psychiatric or general medical exclusion criteria shown in Table 2G and Table 2H below will not be enrolled in the study. No deviations will be permitted from the exclusion criteria.

TABLE 2G
Psychiatric Exclusion Criteria

| No. | Exclusion Criteria |
|---|---|
| 1 | Current or past history of schizophrenia, psychotic |
| | disorder (unless substance induced or due to a |
| | medical condition), bipolar disorder, delusional |
| | disorder, paranoid personality disorder, |
| | schizoaffective disorder, or borderline personality |
| | disorder, or any serious psychiatric comorbidity as |
| | assessed by medical history and structured clinical |
| | interview (version 7.0.2 MINI). |
| 2 | Prior electroconvulsive therapy and/or ketamine for |
| | current episode |
| 3 | Ongoing use of an antidepressant medication, |
| | including augmentation or combination therapies, |
| | other than a single SSRI at Screening (V1) and |
| | Baseline (V2, Day −1). |
| 4 | Current psychological therapies that will not remain |
| | stable within 21 days of the psilocybin session. |
| | Psychological therapies cannot be initiated within 21 |
| | days of baseline. |
| 5 | Current (within the last year) alcohol or substance |
| | abuse as informed by DSM-5 at screening (Visit 1) |
| 6 | Significant suicide risk as defined by (1) suicidal |
| | ideation as endorsed on items 4 or 5 on the C-SSRS |
| | within the past year, at screening or at baseline, or; |
| | (2) suicidal behaviors within the past year, or; (3) |
| | clinical assessment of significant suicidal risk during |
| | subject interview |
| 7 | Depression secondary to other medical conditions. |
| 8 | Other personal circumstances and behavior judged |
| | to be incompatible with establishment of rapport or |
| | safe exposure to psilocybin, including exposure to |
| | psilocybin within the past year and use of |
| | psychedelics, such as ayahuasca, during the current |
| | depressive episode. |

TABLE 2H
General Medical Exclusion Criteria

| No. | Exclusion Criteria |
|---|---|
| 1 | Women who are pregnant, nursing, or planning a |
| | pregnancy. Subjects who are sexually active must |
| | agree to use a highly effective contraceptive method |
| | throughout their participation in the study. Women of |
| | child bearing potential must have a negative urine |
| | pregnancy test at screening (Visit 1) and baseline |
| | (Visit 2) |
| 2 | Cardiovascular conditions: recent stroke (<1 year |
| | from signing of ICF), recent myocardial infarction (<1 |
| | year from signing of ICF), hypertension (blood |
| | pressure >140/90 mmHg or [USA only] QTc >450 |
| | msec) or clinically significant arrhythmia within 1 year |
| | of signing the ICF |
| 3 | Uncontrolled insulin-dependent diabetes. |
| 4 | Seizure disorder. |
| 5 | Positive urine drug screen for illicit drugs or drugs of |
| | abuse (USA only: to include but not limited to |
| | opiates, PCP, cocaine, amphetamines, |
| | methamphetamines, benzodiazepines, barbiturates |
| | and methadone; cannabis for medicinal purposes or |
| | recreational use is permitted) at Visit 1 and Visit 2. |
| | Any positive urine drug test will be reviewed with |
| | subjects to determine the pattern of use and |
| | eligibility will be determined at the investigator's |
| | discretion in conjunction with the MM. |
| 6 | Current enrollment in any investigational drug or |
| | device study or participation in such within 30 days |
| | of screening (Visit 1) |
| 7 | Current enrolment in an interventional study for |
| | depression or participation in such within 30 days of |
| | screening (Visit 1) |
| 8 | Abnormal and clinically significant results on the |
| | physical examination, vital signs, ECG, or laboratory |
| | tests at screening (Visit 1) |
| 9 | Any other clinically significant cardiovascular, |
| | pulmonary, gastrointestinal, hepatic, renal or any |
| | other major concurrent illness that, in the opinion of |
| | the investigator, may interfere with the interpretation |
| | of the study results or constitute a health risk for the |
| | subject if he/she takes part in the study |

### Psilocybin Dose and Administration

Study drug will be administered simultaneously to up to six participants. Each participant will receive 1×25 mg treatment bottle containing 5×5 mg capsules.

After a light breakfast taken 2 h prior to dosing and under observation of research personnel, the 5-capsule dose is to be swallowed with a full glass of water. Due to the number of capsules in a dose, additional water may be necessary to swallow the dose. Research personnel will ensure the entire 5-capsule dose has been swallowed.

To prepare for the drug experience, the subject will take the appropriate dose of psilocybin and lie down on a couch in a room with dim lights and a standard playlist of relaxing music playing quietly. A trained therapist will be present with the subject at all times.

The effects of psilocybin usually start about 20 to 30 min after administration, becoming most intense in the first 90 to 120 min, and gradually subside in 5 to 6 h. The subjects will be asked to remain in the room for the duration of the session regardless of the intensity of the effects, preferably lying down and mostly silent unless they have a concern or need to communicate a discomfort or seek reassurance from the therapist, or use the restroom. The therapist will ‘check-in’ with the subject (i.e., ask how the subject is doing) in 30- to 60-min intervals post-dosing. A light meal and fruit will be available for the subject.

About 5 to 6 h after dosing, trained therapist will discuss the psilocybin experience with the subject. The subject is to be discharged 6 to 8 h post-dosing when, in the opinion of the investigator, the acute effects of psilocybin are resolved. The subject will be accompanied home. The site is to be notified that they have returned home safely, and in the absence of receiving a phone call site staff will directly contact the subject.

### Concomitant Therapy:

All prescription and non-prescription medications (e.g., over-the-counter drugs and herbal supplements) that subjects report taking during the 30 days prior to screening (Visit 1) will be assessed and recorded at that visit. For each medication, documentation should list the trade or generic name, the total daily dose including units (or the dose, units and scheduled and actual frequency of administration if the medication is not taken daily), the route of administration, and the reason for use.

Concomitant medication refers to all drugs and therapies used from the time the informed consent form was signed through the end of study participation.

Changes, additions, or discontinuations to medications will be assessed and recorded during each study visit. All as-needed prescriptions should be converted to reflect actual number of pills or dose taken per day.

### Permissible Medications:

Participants must have been receiving treatment with a SSRI (fluoxetine, fluvoxamine, sertraline, paroxetine, vilazadone, vortioxetine, citalopram or escitalopram) at an adequate dose according to local recommendations for at least 6 weeks before Screening (V1) and Baseline (V2, Day −1). Dose changes within the adequate range are acceptable. We recommend that the participant is continued on the SSRI for the duration of the follow-up period of the study, ideally at the same dose.

Medications for the management of concurrent anxiety and insomnia, or nonpsychiatric medications that have a potential psychotropic effect are permitted within the following limitations. From the initial Screen Visit (V1) through final study visit (V7, EOS), participants are permitted to use benzodiazepines (up to 2 mg of lorazepam equivalents per day) for insomnia and anxiety if it is not taken within 12 h before the psilocybin dose. Prescription and nonprescription medications with psychoactive properties that are used as needed for nonpsychiatric conditions (e.g., pseudoephedrine for allergies or cold symptoms; zopiclone for sleep disorders) should be used no more than two times a week and not in the 12 h before any study assessment. Documentation of the use of adjunctive anxiolytics, hypnotics or medication with potential psychotropic properties (including OTC preparations) will be obtained at each clinic visit.

Therapy considered necessary for the subject's welfare may be given at the discretion of the study clinician.

### Adverse Events

Throughout the course of the study, all adverse events (AEs) will be monitored and recorded and will include the AE's description, start and end date, seriousness, severity, action taken, and relationship to the treatment. If AEs occur, the first concern will be the safety of the study subjects.

An AE is any untoward medical occurrence in a subject administered a pharmaceutical product and which does not necessarily have to have a causal relationship with this treatment. An AE can therefore be any unfavorable and unintended sign (including an abnormal laboratory finding, for example), symptom, or disease temporally associated with the use of a medicinal product, whether considered related to the medicinal product.

Medical interventions such as surgeries, diagnostic procedures, and therapeutic procedures are not AEs but the action taken to treat the medical condition. They should be recorded as treatment of the AEs.

### Efficacy and Outcome Measures:

Efficacy and outcome measures include MADRS, CGI-S, CGI-I, EQ-5D-3L (subject and caregiver), QIDS-SR-16, GAD-7, PANAS, 5D-ASC, and C-SSRS.

### Analysis of Safety:

Safety analyses will be performed using data from the safety population. Safety will be evaluated based of AEs, vital signs, clinical laboratory assessments, and ECG findings.

### Columbia-Suicide Severity Rating Scale (C-SSRS)

Item scores from the C-SSRS, all visits by randomized treatment, the item scores from the version assessing suicidality since the last visit, and all postbaseline visits (Visit 3 to Visit 7, inclusive) by treatment will be tabulated. Summary statistics of suicidal ideation and suicidal behavior following psilocybin administration will be presented by randomized treatment.

### Adverse Events

AEs will be coded by Preferred Term (PT) using the Medical Dictionary for Regulatory Activities (MedDRA) classification. All reported AEs with onset or worsening after the administration of study medication will be included in the analysis. The incidence of AEs will be summarized by treatment group, and by severity and relationship to psilocybin. Serious AEs and AEs leading to withdrawal from the study will be tabulated.

A TEAE is defined as any AE that has an onset on or after the dose of psilocybin, or any pre-existing condition that has worsened on or after the dose of psilocybin.

The incidence of TEAEs and treatment-related AEs will also be summarized by maximum severity and most-related relationship to psilocybin by MedDRA primary system organ class and PT. The summary will include the total number and percentage of subjects reporting an event. In counting the number of events reported, a continuous event, i.e., reported more than once and which did not cease, will be counted only once; non continuous AEs reported several times by the same subject will be counted as multiple events.

### Electrocardiographic Data

The ECG data will be summarized descriptively based on measures of change in each ECG parameter from Visit 1 to post-treatment (Visit 4). Frequency tabulations of the abnormalities will be provided. ECG variables to be analyzed will include heart rate, PR interval, QRS interval, QT interval, and corrected QT interval using the following correction methods: QT corrected according to Bazett's formula and QT corrected according to Fridericia's formula.

### Laboratory Data

Laboratory data (hematology and blood chemistry parameters) will be presented for each treatment group using descriptive statistics, including mean and mean change from baseline values at each scheduled time point. Shift tables will display numbers of subjects with normal/abnormal values at Baseline versus post-treatment. The frequency of laboratory abnormalities will be tabulated. By-subject data listings will flag laboratory values that are outside normal reference ranges or markedly abnormal findings.

### Vital Signs

Changes from baseline in vital signs, blood pressure (systolic and diastolic), body temperature, pulse rate, and respiratory rate, will be summarized for each treatment group using descriptive statistics. The last measurement obtained prior to treatment administration will serve as baseline. The percentage of subjects with values outside clinically important limits will be summarized. A listing of weight and height at Visit 1 will be provided.

### Demographic and Baseline Characteristics

Treatment groups will be compared with respect to subject demographics and baseline characteristics will be summarized using descriptive statistics, no formal statistical analysis tests will be performed. Table 3 shows demographics of the subjects and Table 4 shows the baseline depression of the subjects.

TABLE 3
Demographic Characteristics of the Subjects

| | | |
|---|---|---|
| | | COMP360 |
| | | 25 mg + SSRI |
| Parameter | Statistic | N = 19 |

Gender

| | | |
|---|---|---|
| Male | n (%) | 6 (31.6) |
| Female | n (%) | 13 (68.4) |

BMI at screening (kg/m²)

| | |
|---|---|
| Mean (SD) | 26.26 (6.52) |
| Min, Max | 18.6, 45.5 |

Age at screening (years)

| | |
|---|---|
| Mean (SD) | 42.2 (10.8) |
| Min, Max | 21, 64 |

Age at screening (years) group

| | | |
|---|---|---|
| 18-34 | n (%) | 4 (21.1) |
| 35-64 | n (%) | 15 (78.9) |
| 65-84 | n (%) | 0 |
| >84 | n (%) | 0 |

Race

| | | |
|---|---|---|
| White | n (%) | 15 (78.9) |
| Black or African American | n (%) | 2 (10.5) |
| Other | n (%) | 2 (10.5) |

Ethnicity

| | | |
|---|---|---|
| Not Hispanic or Latino | n (%) | 19 (100) |

TABLE 4
baseline depression of the subjects

| | | |
|---|---|---|
| | | COMP360 |
| | | 25 mg + SSRI |
| Parameter | Statistic | N = 19 |

Baseline MADRS total score

| | |
|---|---|
| Mean (SD) | 31.7 (5.77) |
| Min, Max | 18, 39 |

Baseline HAM-D-17 total score

| | |
|---|---|
| Mean (SD) | 20.2 (2.80) |
| Min, Max | 17, 28 |

HAM-D-17 baseline severity categories

| | | |
|---|---|---|
| Moderate (18-23) | n (%) | 16 (88.9) |
| Severe (≥24) | n (%) | 2 (11.1) |

### Results:

In the trial, a single dose of 25 mg of psilocybin was given to 19 patients as an adjustive to SSRI therapy and in conjunction with psychological support from specially trained therapists.

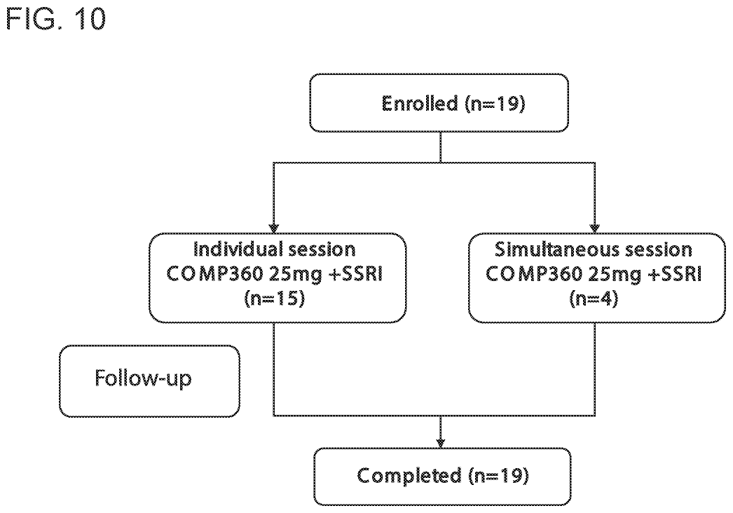

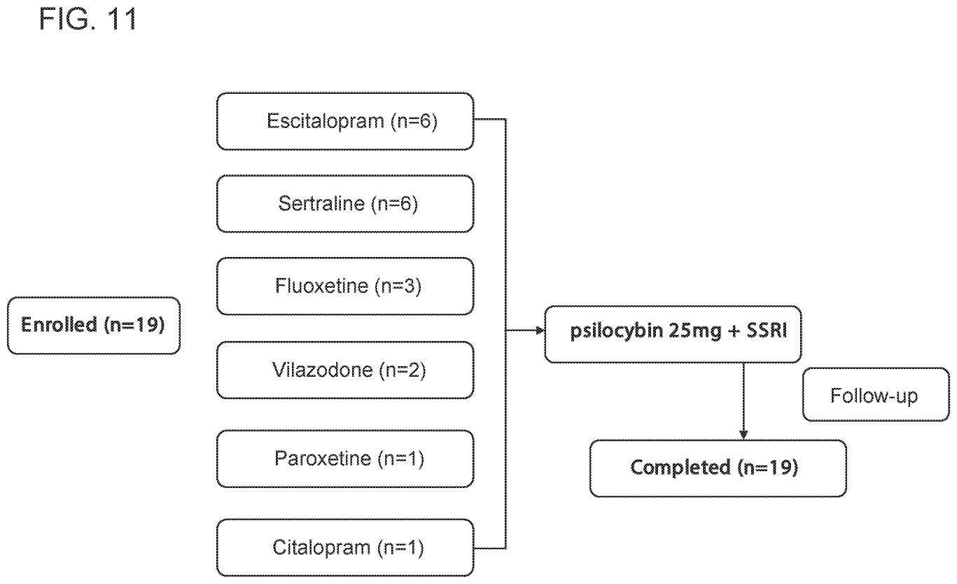

FIG. 10 shows the patient disposition from the clinical trial. FIG. 11 shows the SSRI that participants were taking on the day psilocybin was administered.

### Efficacy

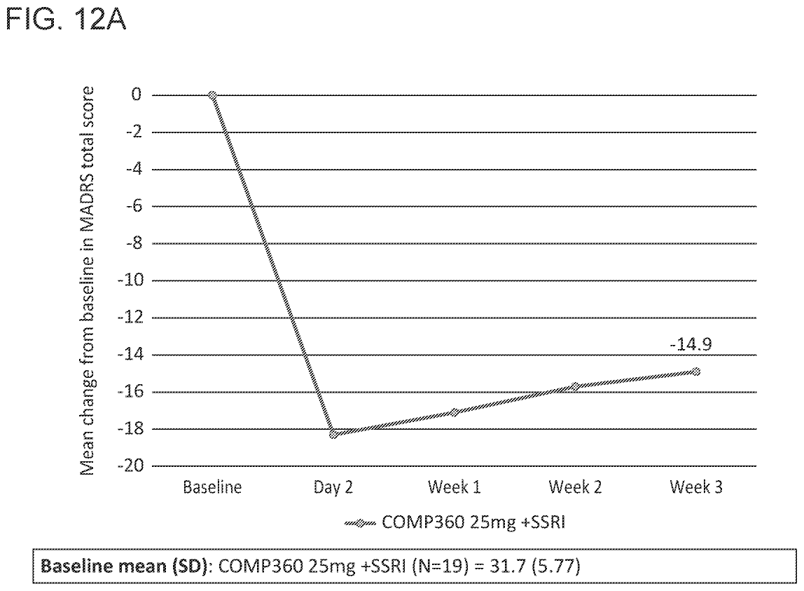

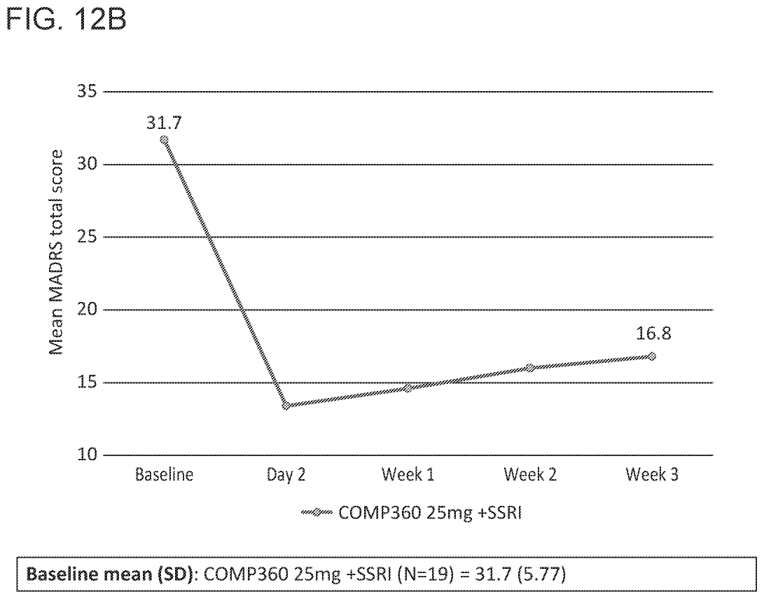

FIGS. 12A-12B show the mean change from baseline in MADRS total score and the mean MADRS total score, respectively, by visit. Participants on average had a 14.9 reduction in their MADRS total score at week 3 from baseline. Patients administered a combination of psilocybin and an SSRI exhibited a greater reduction in their MADRS score as compared to patients administered only psilocybin. Rapid onset of action. No participants started new treatment for depression during the 3 weeks follow-up. Table 5 shows the MADRS total score summary by visit.

TABLE 5
MADRS Total Score Summary by Visit

| | |
|---|---|
| | Psilocybin |
| | 25 mg + SSRI |
| Visit / Statistic | N = 19 |

Baseline

| | |
|---|---|
| n | 19 |
| Mean (SD) | 31.7 (5.77) |
| (Min, Max) | (18, 39) |

Day 2

| | |
|---|---|
| n | 19 |
| Mean (SD) | 13.4 (12.92) |
| (Min, Max) | (0, 39) |
| Change from baseline Mean (SD) | −18.3 (11.85) |
| (Min, Max) | (−37, 6) |

Week 1

| | |
|---|---|
| n | 19 |
| Mean (SD) | 14.6 (11.88) |
| (Min, Max) | (1, 41) |
| Change from baseline Mean (SD) | −17.1 (11.29) |
| (Min, Max) | (−34, 4) |

Week 2

| | |
|---|---|
| n | 19 |
| Mean (SD) | 16.0 (11.70) |
| (Min, Max) | (2, 40) |
| Change from baseline Mean (SD) | −15.7 (12.02) |
| (Min, Max) | (−35, 4) |

Week 3

| | |
|---|---|
| n | 19 |
| Mean (SD) | 16.8 (11.62) |
| (Min, Max) | (1, 41) |
| Change from baseline Mean (SD) | −14.9 (11.97) |
| (Min, Max) | (−32, 5) |

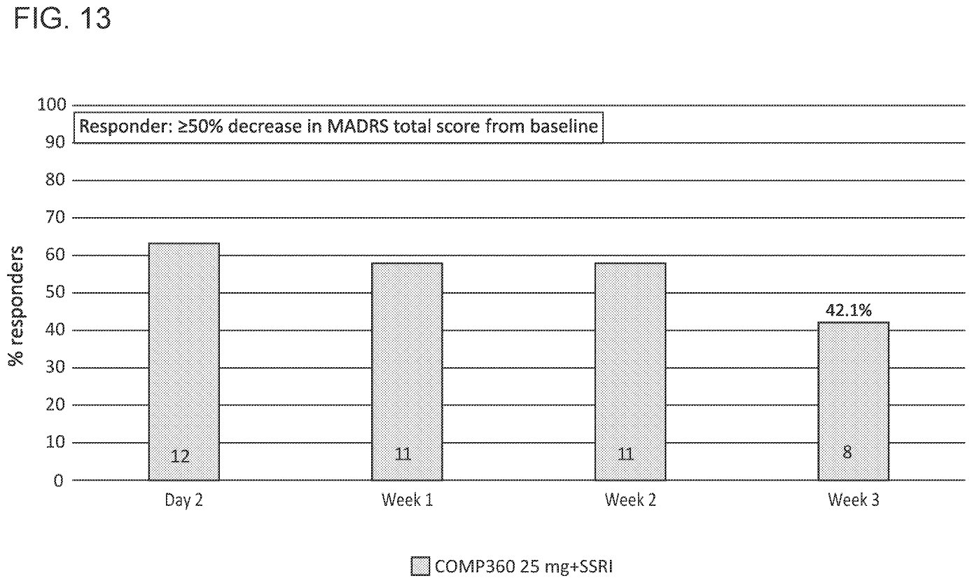

FIG. 13 shows the proportion of MADRS responders by visit (Responder: ≥50% decrease in MADRS total score from baseline) by visit. There was a rapid response was observed from day 2 and ~42% of participants were MADRS responders at week 3. Table 6 shows the number of MADRS responders at Day 2, Week 1, Week 2 and Week 3.

TABLE 6
MADRS Responders at Day 2, Week 1, Week 2 and Week 3

| | |
|---|---|
| | COMP360 |
| | 25 mg + SSRI |
| Visit / Statistic | N = 19 |

Day 2

| | |
|---|---|
| n (%) | 12 (63.2) |

Week 1

| | |
|---|---|
| n (%) | 11 (57.9) |

Week 2

| | |
|---|---|
| n (%) | 11 (57.9) |

Week 3

| | |
|---|---|
| n (%) | 8 (42.1) |

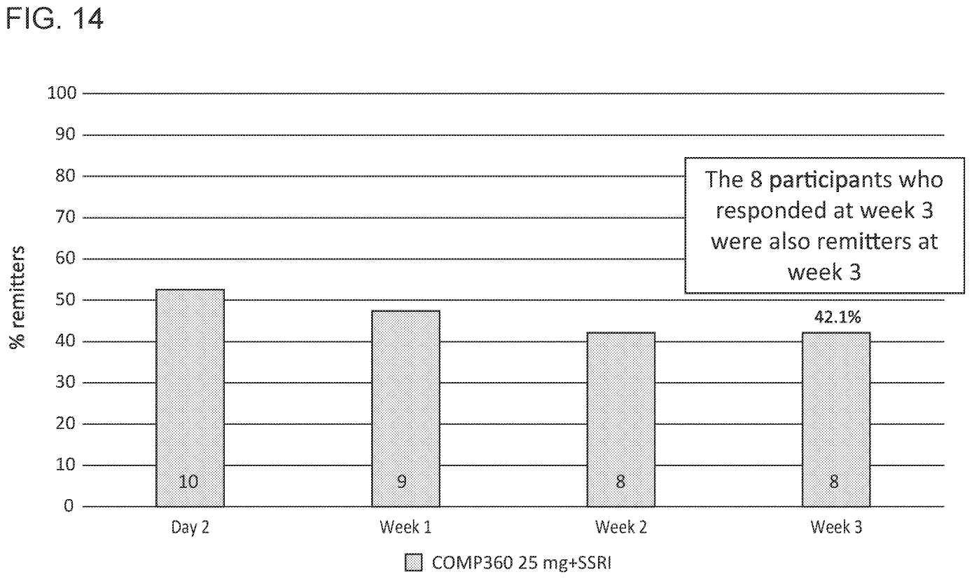

FIG. 14 shows the proportion of MADRS remitters by visit observed during the study. Rapid remission was observed from day 2 and ~42% participants were MADRS remitters by week 3. Table 7 shows the number of MADRS remitters at Day 2, Week 1, Week 2 and Week 3.

TABLE 7
The Number of MADRS Remitters at
Day 2, Week 1, Week2, and Week 3

| | |
|---|---|
| | COMP360 |
| | 25 mg + SSRI |
| Visit / Statistic | N = 19 |

Day 2

| | |
|---|---|
| n (%) | 10 (52.6) |

Week 1

| | |
|---|---|
| n (%) | 9 (47.4) |

Week 2

| | |
|---|---|
| n (%) | 8 (42.1) |

Week 3

| | |
|---|---|
| n (%) | 8 (42.1) |

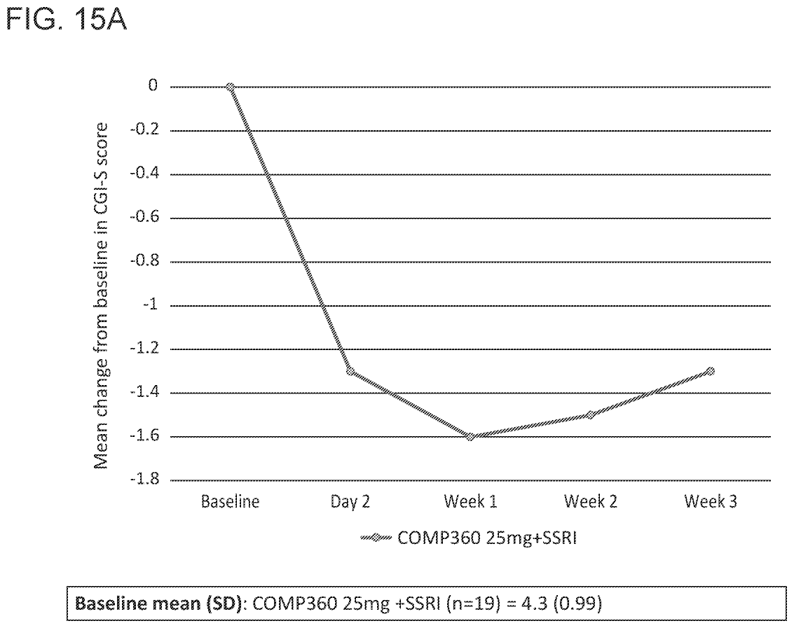

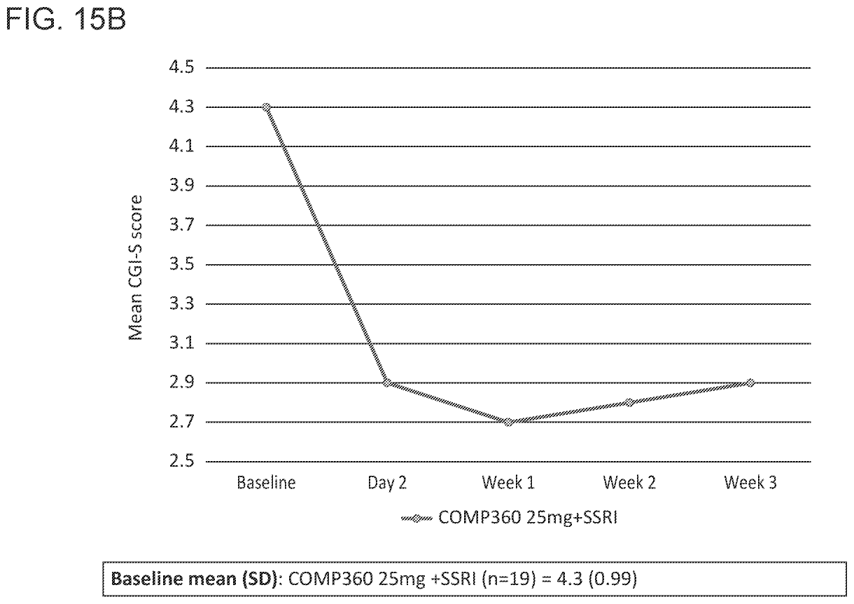

FIG. 15A shows the mean change from baseline in Clinical Global Impression-Severity scale (CGI-S) total score observed by visit. FIG. 15B shows the mean CGI-S total score observed by visit. There was an apparent rapid reduction in severity of depression according to clinical judgment. On average, participants scored 4 “moderately ill” in the CGI-S at baseline and this reduced to 3 “mildly ill” at week 3.

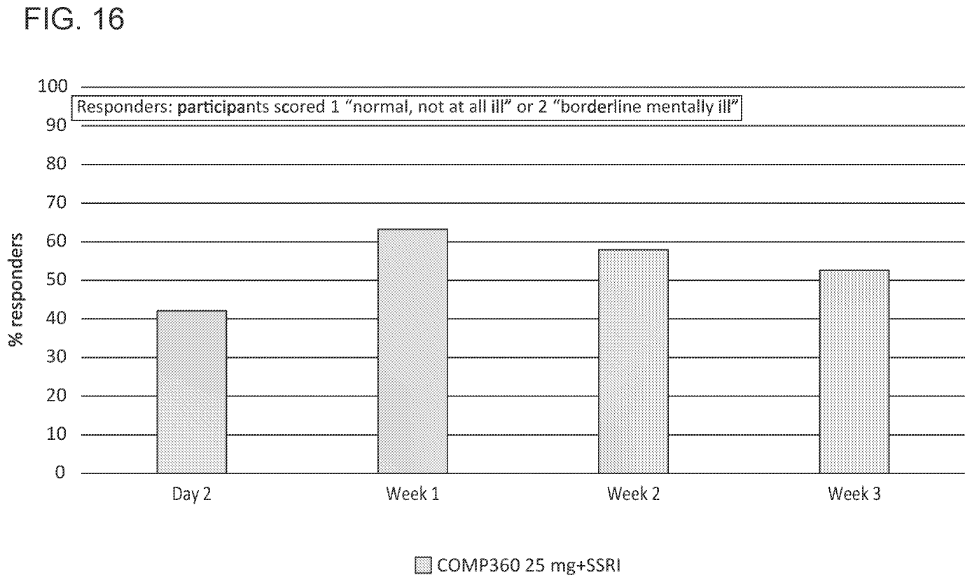

FIG. 16 shows the proportion of CGI-S responders by visit. Rapid response was observed from day 2 and 52.6% of participants were responders at week 3. Table 8 shows the number of CGI-S responders at Day 2, Week 1, Week 2 and Week 3.

TABLE 8
The Number of CGI-S Responders at
Day 2, Week 1, Week 2 and Week 3.

| | |
|---|---|
| | COMP360 |
| | 25 mg + SSRI |
| Visit / Statistic | N = 19 |

Day 2

| | |
|---|---|
| n (%) | 8 (42.1) |

Week 1

| | |
|---|---|
| n (%) | 12 (63.2) |

Week 2

| | |
|---|---|
| n (%) | 11 (57.9) |

Week 3

| | |
|---|---|
| n (%) | 10 (52.6) |

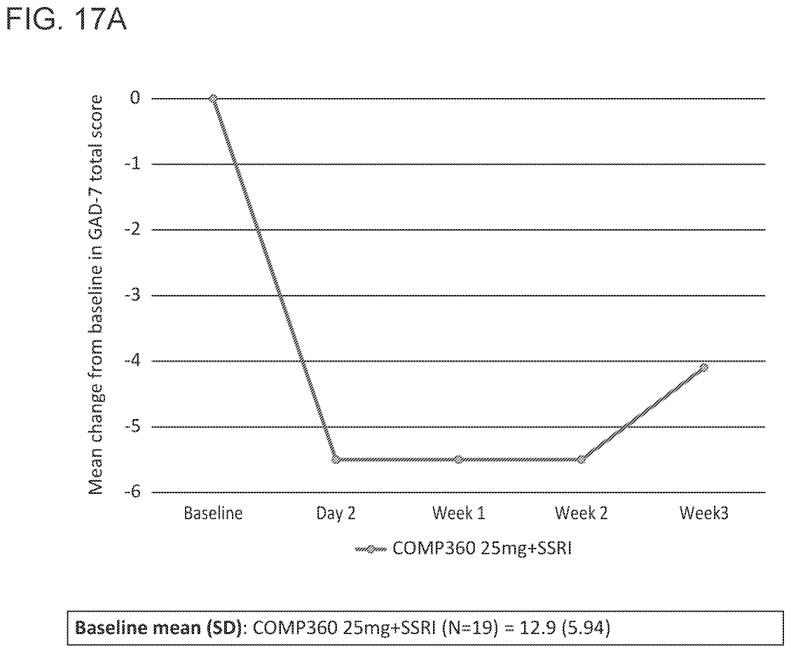

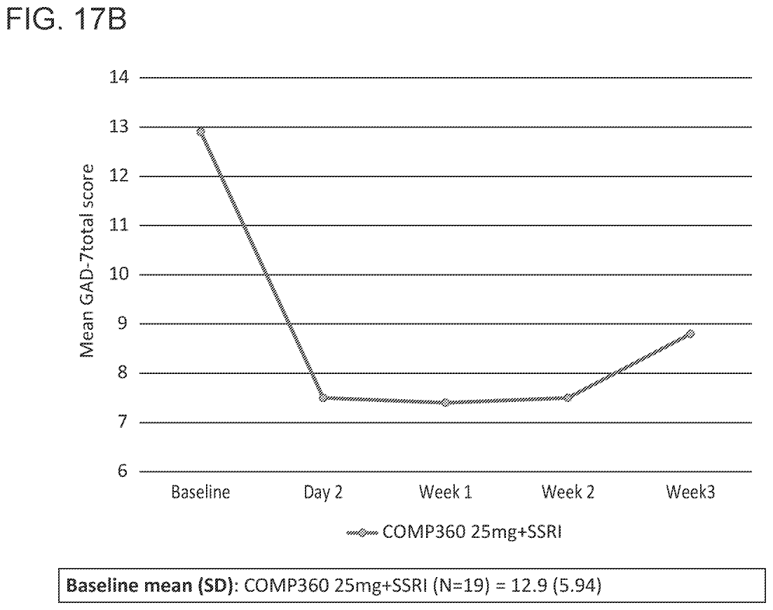

FIG. 17A shows the mean change from baseline in Generalised Anxiety Disorder-7 item scale (GAD-7) total score by visit. FIG. 17B shows the mean GAD-7 total score observed by visit. There was a clear reduction of anxiety symptoms.

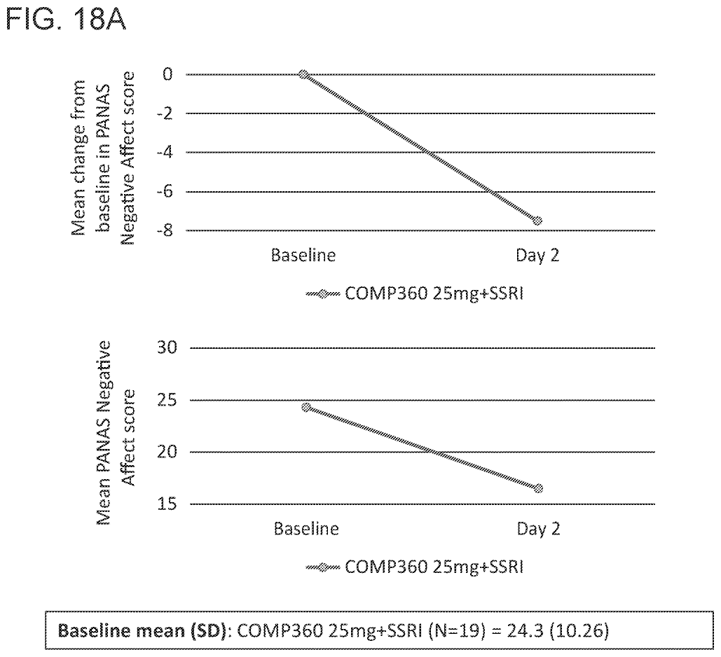

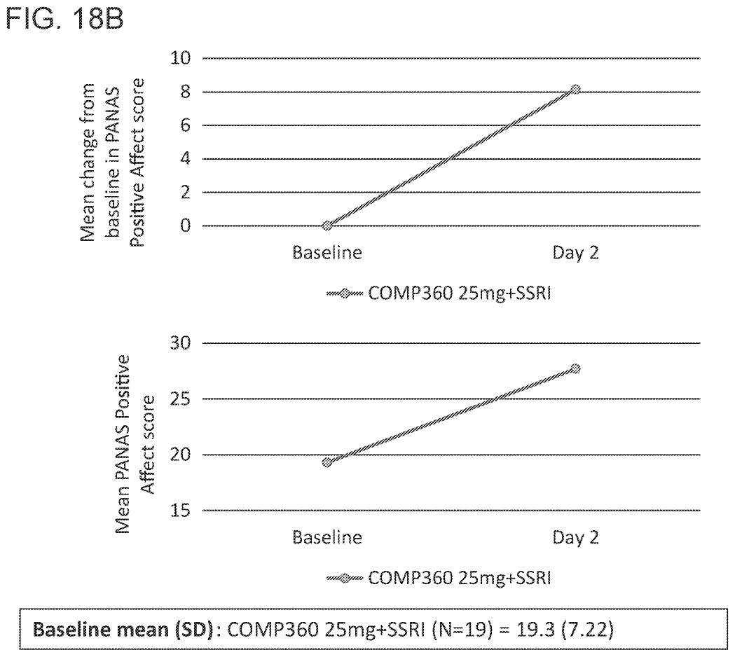

FIG. 18A shows the mean change from baseline in Positive and Negative Affect Schedule (PANAS) Negative Affect total score and the mean PANAS Negative Affect total score by visit. FIG. 18B shows the mean change from baseline in PANAS Positive Affect total score and the mean PANAS Positive Affect total score by visit. Higher Positive Affect and lower Negative Affect changes from baseline found at day 2.

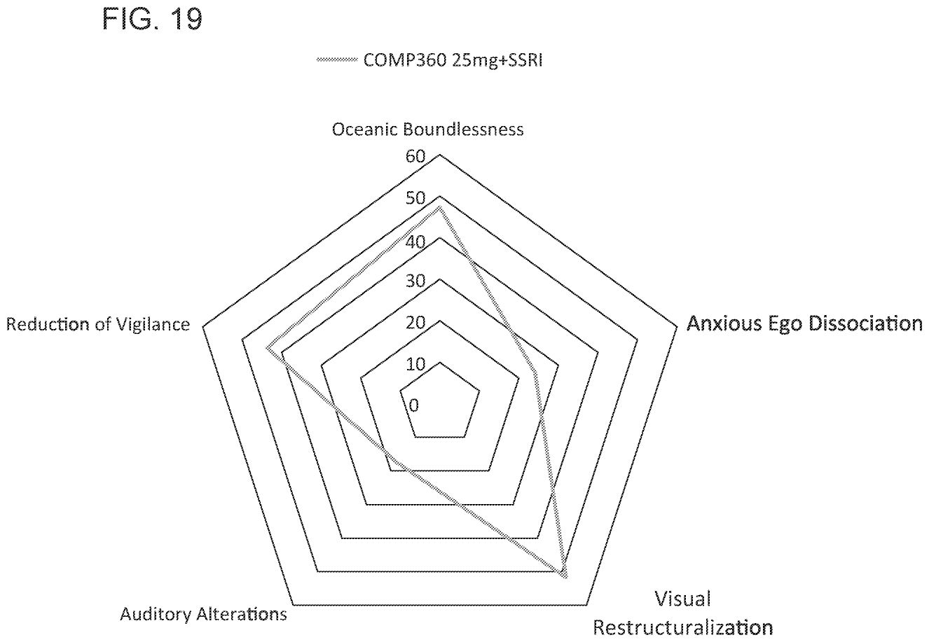

FIG. 19 shows the summary of the 5-Dimensional Altered States of Consciousness scale (5D-ASC) at day 1 observed during the clinical study described in Example 2.

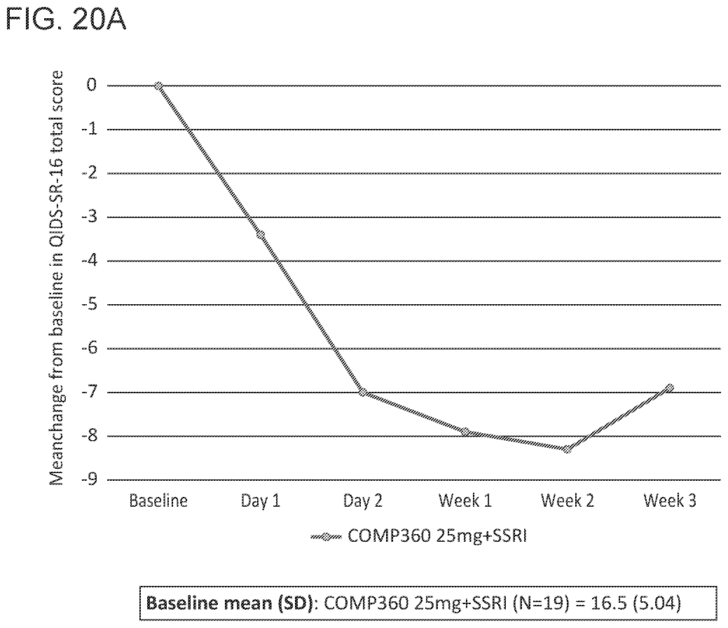

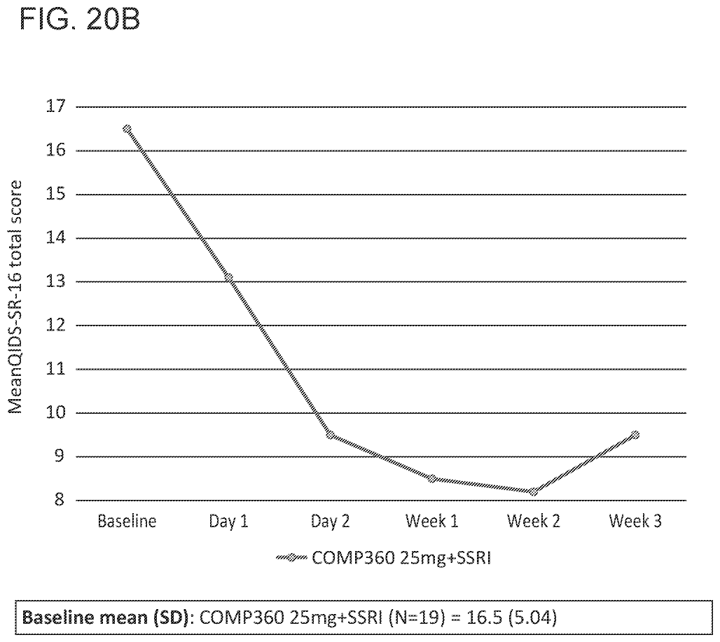

FIG. 20A shows the mean change from baseline in 16-Item Quick Inventory of Depressive Symptomatology-Self Report (QIDS-SR-16) total score by visit. Expected findings in line with pharmacological profile of psilocybin in previous studies found on all primary dimensions. FIG. 20B shows the mean QIDS-SR-16 total score by visit. QIDS-SR-16 shows a similar pattern to MADRS data with a rapid reduction of self-reported depression symptoms from day 2.

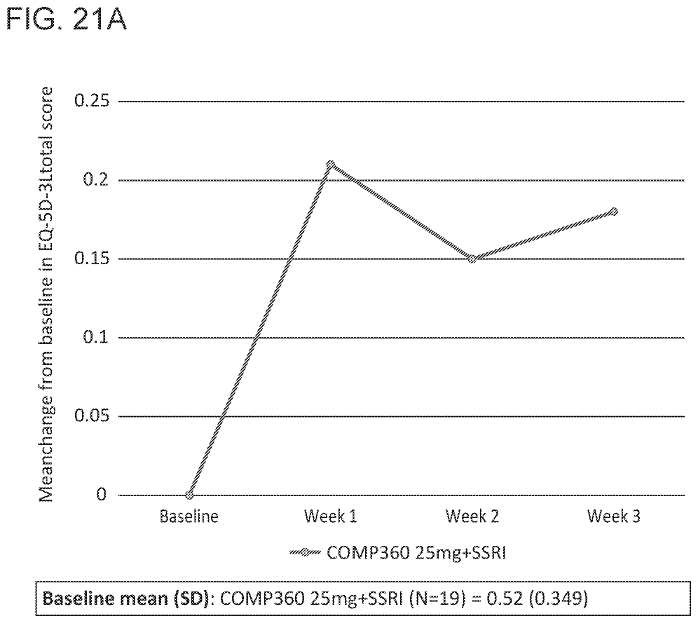

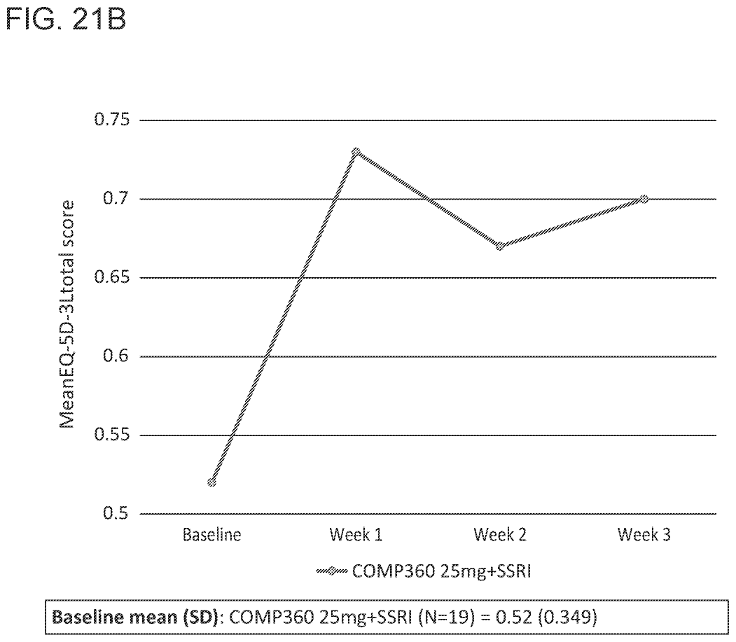

FIG. 21A shows the mean change from baseline in EuroQol-5-Dimension-3-Level Scale (EQ-5D-3L) total score observed during the clinical study described in Example 2. FIG. 21B shows the mean EQ-5D-3L total score by visit.

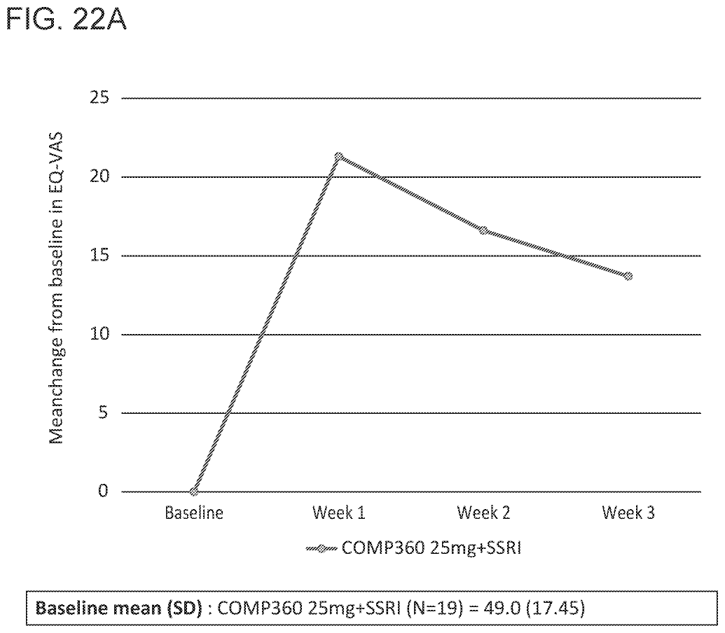

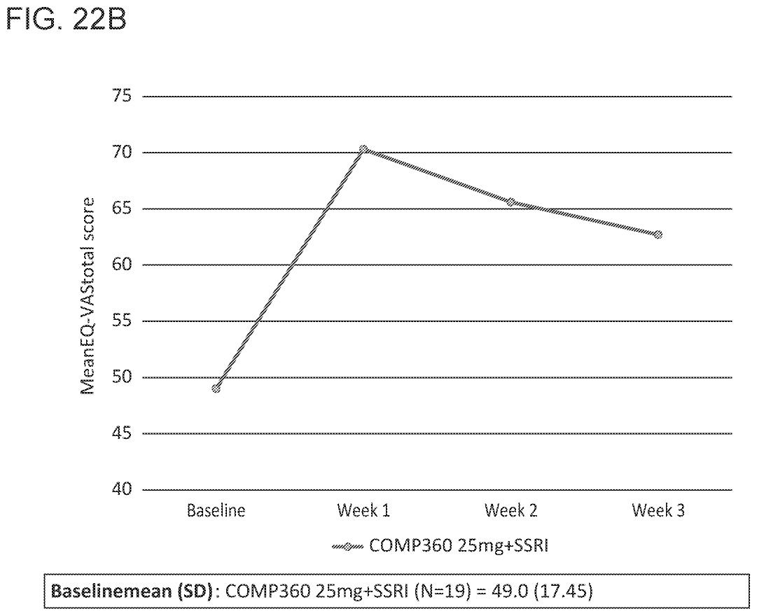

FIG. 22A shows the mean change from baseline in Euro Qol visual analog scale (EQ-VAS) total score by visit. FIG. 22B shows the mean EQ-VAS total score by visit. Participants reported to have an overall better health at week 3.

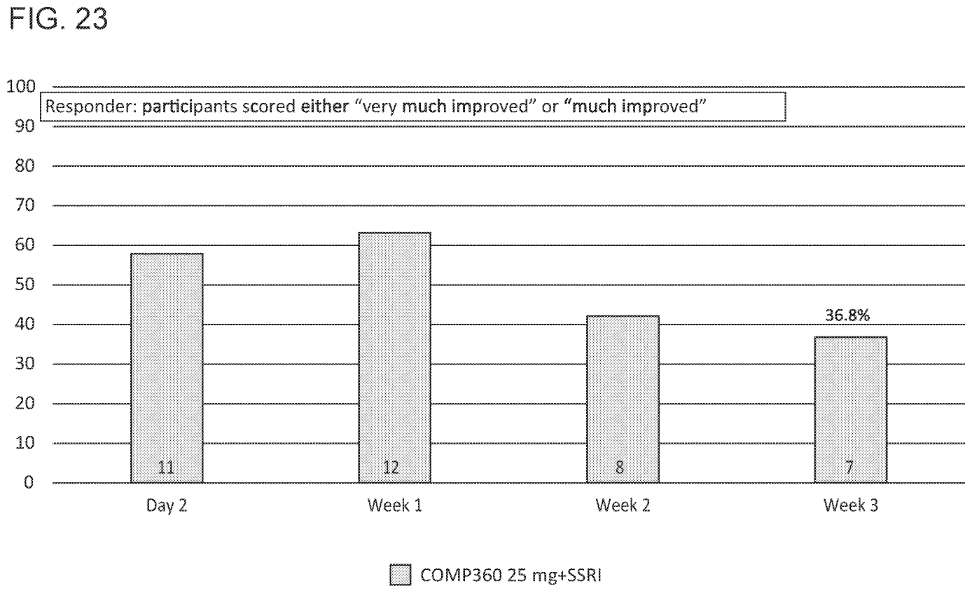

FIG. 23 shows the proportion of Clinical Global Impression-Improvement scale (CGI-I) responders by visit. Participants reported to have an overall better health at week 3. Table 9 shows the number of CGI-I responders at Day 2, Week 1, Week 2 and Week 3.

TABLE 9
The Number of CGI-I Responders at
Day 2, Week 1, Week 2 and Week 3.

| | |
|---|---|
| | COMP360 |
| | 25 mg + SSRI |
| Visit / Statistic | N = 19 |

Day 2

| | |
|---|---|
| n (%) | 11 (57.9) |

Week 1

| | |
|---|---|
| n (%) | 12 (63.2) |

Week 2

| | |
|---|---|
| n (%) | 8 (42.1) |

Week 3

| | |
|---|---|
| n (%) | 7 (36.8) |

### Safety:

COMP360 was generally well tolerated. In the study, 11 participants (58%) reported at least one treatment emergent adverse event (TEAE). The most common TEAE was headache (6 participants with 6 events), occurring on the day of dosing or the day after and all cases resolved in one or two days. Table 10 shows the treatment emergent adverse events by group.

TABLE 10
Treatment Emergent Adverse Events by Group

COMP360
25 mg + SSRI
N = 19

| MedDRA TEAE Preferred Term | n (%) | Severity |
|---|---|---|
| Headache | 5 (26.3) | mild |
| | 1 (5.3) | moderate |
| Blood pressure increase | 1 (5.3) | mild |
| Depression | 1 (5.3) | moderate |
| Diarrhoea | 1 (5.3) | mild |
| Dizziness | 1 (5.3) | mild |
| Dry mouth | 1 (5.3) | mild |
| Fall | 1 (5.3) | mild |
| Hypertension | 1 (5.3) | severe |
| Palpitations | 1 (5.3) | mild |
| Skin abrasion | 1 (5.3) | mild |
| Vertigo | 1 (5.3) | mild |

MedDRA = Medical Dictionary for Regulatory Activities; TEAE = treatment emergent adverse event; N = number of participants in the population; n = number observed

In the study, there were no TEAEs classed as serious (TESAEs) and no TEAEs related to suicidal ideation or behaviour or intentional self-injury.

All, documents, patents, patent applications, publications, product descriptions, and protocols which are cited throughout this application are incorporated herein by reference in their entireties for all purposes.

The embodiments illustrated and discussed in this specification are intended only to teach those skilled in the art the best way known to the inventors to make and use the invention. Modifications and variation of the above-described embodiments of the invention are possible without departing from the invention, as appreciated by those skilled in the art in light of the above teachings. It is therefore understood that, within the scope of the claims and their equivalents, the invention may be practiced otherwise than as specifically described.

The foregoing is illustrative of the present invention, and is not to be construed as limiting thereof. The invention is defined by the following claims, with equivalents of the claims to be included therein.

1. A method of treating treatment-resistant depression in a subject in need thereof, the method comprising administering an effective amount of psilocybin or an active metabolite thereof to the subject as an adjunctive to Selective Serotonin Reuptake Inhibitor (SSRI) therapy.

2. The method of claim 1, wherein the SSRI is selected from the group consisting of escitalopram, sertraline, fluoxetine, vilazodone, vortioxetine, paroxetine and citalopram.

3. The method of claim 1, wherein the SSRI is administered prior to administration of psilocybin.

4. The method of claim 1, wherein the SSRI is administered after administration of psilocybin.

5. The method of claim 1, wherein the SSRI is administered on the same day as the psilocybin.

6. The method of claim 1, wherein about 25 mg of psilocybin or an active metabolite thereof is administered to the subject.

7. The method of claim 1, wherein at least one sign or symptom of depression is reduced by the administration of psilocybin.

8. The method of claim 7, wherein the sign or symptom of depression is depressed mood, diminished interest in activities, weight loss or gain, decrease or increase in appetite, insomnia or hypersomnia, psychomotor agitation or retardation, fatigue or loss of energy, feelings of worthlessness or excessive or inappropriate guilt, diminished ability to concentrate or indecisiveness, or suicidal ideation or behavior.

9. The method of claim 7, wherein the sign or symptom of depression is measured according to a diary assessment, an assessment by clinician or caregiver, a clinical rating scale, or by functional MRI.

10. The method of claim 9, wherein the clinical rating scale is a Quick Inventory of Depressive Symptomatology (QIDS)-16 scale, a QIDS-16 daily scale, a Hamilton Depression Rating scale, a Beck Depression Inventory scale, a Montgomery-Åsberg Depression Rating Scale (MADRS), a Clinical Global Impression Scale, a Zung Self-Rating Depression Scale, a Raskin Depression Rating Scale, Young Mania Rating Scale, or combinations thereof.

11. The method of claim 9, wherein the clinical rating scale is MADRS.

12. The method of claim 11, wherein the administration of psilocybin provides an about 50% reduction in patient's MADRS total score compared to prior to treatment with psilocybin.

13. The method of claim 11, wherein the administration of psilocybin provides a reduction in the patient's MADRS total score to ≤10.

14. The method of claim 9, wherein the clinical rating scale is Clinical Global Impression-Severity scale (CGI-S).

15. The method of claim 14, wherein the administration of psilocybin provides a reduction in the patient's CGI-S score to ≤2.

16. The method of claim 9, wherein the clinical rating scale is Clinical Global Impression-Improvement scale (CGI-I).

17. The method of claim 16, wherein the administration of psilocybin provides a reduction in the patient's CGI-I score reported as “very much improved” or “much improved” compared to prior to treatment with psilocybin.

18. The method of claim 1, wherein the administration of psilocybin provides a reduction in the patient's anxiety compared to prior to treatment with psilocybin.

19. The method of claim 18, wherein the administration of psilocybin provides a reduction in the patient's Generalised Anxiety Disorder-7 item scale (GAD-7) total score compared to prior to treatment with psilocybin.

20. The method of claim 1, wherein at least one sign or symptom of depression is alleviated within 24 hours of administration of the psilocybin.

21. The method of claim 1, wherein at least one symptom of depression is alleviated within 1 week of administration of the psilocybin.

22. The method of claim 1, wherein at least one symptom of depression is alleviated for a period of at least 1 month, at least 3 months, or at least 12 months after administration of the psilocybin.

23. (canceled)

24. (canceled)

25. The method of claim 1, wherein at least two doses of psilocybin are administered to the subject.

26. The method of claim 1, wherein the psilocybin is administered once per day.

27. The method of claim 25, wherein the psilocybin is administered at least once or twice per week.

28. (canceled)

29. The method of claim 25, wherein the psilocybin is administered at least once per month, at least twice per month, at least once every three months, at least once every six months, or at least once every 12 months.

30. (canceled)

31. (canceled)

32. (canceled)

33. (canceled)

34. The method of claim 25, wherein each dose of psilocybin administered is about 25 mg.

35. The method of claim 1, wherein the psilocybin is administered by one of the following routes: oral, intravenous, intramuscular, parenteral, topical, inhalation, rectal, transmucosal, intranasal, buccal, vaginal, intrathecal, intraocular, transdermal, in utero, intralymphatic, or by direct tissue or organ injection.

36. The method of claim 35, wherein the psilocybin is administered orally.

37. The method of claim 1, wherein the psilocybin comprises a crystalline psilocybin.

38. (canceled)

39. (canceled)

40. (canceled)

41. (canceled)

42. (canceled)

43. (canceled)

44. (canceled)

45. (canceled)

46. The method of claim 37, wherein the crystalline psilocybin is characterized by an X-ray powder diffraction (XRPD) peak at 17.5±0.1 and one or more XRPD peaks selected from 11.5±0.1, 12.0±0.1, 14.5±0.1, and 19.7±0.1 °2θ.

47. The method of claim 37, wherein the crystalline psilocybin is characterized by an X-ray powder diffraction (XRPD) peak at 19.7±0.1 and one or more XRPD peaks selected from 11.5±0.1, 12.0±0.1, 14.5±0.1, and 17.5±0.1 °2θ.
